SEQUENCE LISTING (2 sequences)

SEQ ID NO: 1 - DNA, 1794 bp, Homo sapiens
  source 1..1794: mol_type = genomic DNA
atgggcaagaagcacaagaagcacaaggccgagtggcgctcgtcctacgaggattatgccgacaagcccctggagaagcctctaaagctagtcctgaaggtcggaggaagtgaagtgactgaactctcaggatccggccacgactccagttactatgatgacaggtcagaccatgagcgagagaggcacaaagaaaagaaaaagaagaagaagaagaagtccgagaaggagaagcatctggacgatgaggaaagaaggaagcgaaaggaagagaagaagcggaagcgagagagggagcactgtgacacggagggagaggctgacgactttgatcctgggaagaaggtggaggtggagccgcccccagatcggccagtccgagcgtgccggacacagccagccgaaaatgagagcacacctattcagcaactcctggaacacttcctccgccagcttcagagaaaagatccccatggattttttgcttttcctgtcacggatgcaattgctcctggatattcaatgataataaaacatcccatggattttggcaccatgaaagacaaaattgtagctaatgaatacaagtcagttacggaatttaaggcagatttcaagctgatgtgtgataatgcaatgacatacaataggccagataccgtgtactacaagttggcgaagaagatccttcacgcaggctttaagatgatgagcaaacaggcagctcttttgggcaatgaagatacagctgttgaggaacctgtccctgaagttgtaccagtacaagtagaaactgccaagaaatccaaaaagccgagtagagaagttatcagctgcatgtttgagcctgaagggaatgcctgcagcttgacggacagtaccgcagaggagcacgtgctggcgctggtggagcacgcagctgacgaagctcgggacaggatcaaccggttcctcccaggcggcaagatgggctatctgaagaggaacggggacgggagcctgctctacagcgtggtcaacacggccgagccggacgctgatgaggaggagacccacccggtggacttgagctcgctctccagtaagctactcccaggcttcaccacgctgggcttcaaagacgagagaagaaacaaagtcacctttctctccagtgccactactgcgctttcgatgcagaataattcagtatttggcgacttgaagtcggacgagatggagctgctctactcagcctacggagatgagacaggcgtgcagtgtgcgctgagcctgcaggagtttgtgaaggatgctgggagctacagcaagaaagtggtggacgacctcctggaccagatcacaggcggagaccactctaggacgctcttccagctgaagcagagaagaaatgttcccatgaagcctccagatgaagccaaggttggggacaccctaggagacagcagcagctctgttctggagttcatgtcgatgaagtcctatcccgacgtttctgtggatatctccatgctcagctctctggggaaggtgaagaaggagctggaccctgacgacagccatttgaacttggatgagacgacgaagctcctgcaggacctgcacgaagcacaggcggagcgcggcggctctcggccgtcgtccaacctcagctccctgtccaacgcctccgagagggaccagcaccacctgggaagcccttctcgcctgagtgtcggggagcagccagacgtcacccacgacccctatgagtttcttcagtctccagagcctgcggcctctgccaagacctaa

SEQ ID NO: 2 - protein, 597 aa, Homo sapiens
MGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKT

(19) United States
(12) Patent Application Publication
(10) Pub. No.: US 2026/0224559 A1
(43) Pub. Date: Aug. 6, 2026

(54) FORMULATIONS FOR TREATING CANCER

(71) Applicant: FOGHORN THERAPEUTICS INC., Cambridge, MA (US)

(72) Inventors: Abira RAMAKRISHNAN, Cambridge, MA (US); Chong-Hui GU, Waban, MA (US)

(21) Appl. No.: 19/152,550

(22) PCT Filed: Feb. 1, 2024
(86) PCT No.: PCT/US2024/014021

§ 371 Date: Jul. 31, 2025

Related U.S. Application Data
(60) Provisional application No. 63/482,771, filed on Feb. 1, 2023.

(51) Int. Cl. A61K 31/496; A61K 9/00; A61K 9/10; A61K 47/54; A61K 47/55; A61P 35/00

(52) U.S. Cl. CPC A61K 31/496; A61K 9/0019; A61K 9/10; A61K 47/545; A61K 47/55; A61P 35/00

(57) ABSTRACT

The present disclosure relates to methods and compositions for the treatment of cancer. Further disclosed pharmaceutically acceptable composition comprising a long-acting formulation of a conjugate of Targeting Ligand and Targeting Ligase Binder, where the targeting ligase binder is an E3 ubiquitin ligase binding moiety, VHL E3 ubiquitin ligase binding moiety, MDM2 E3 ligase binding moiety, or IAP E3 ubiquitin ligase binding moiety, and Targeting Ligand binds to BRD9, CBP, EP300, estrogen receptor, androgen receptor, IRAK4, IRAK4 and/or IMiD, STAT3, MDM2, BCLXL, BTK, BTK and/or IMiD, TRK, BRM, IKZF1 and/or IKZF3, BRAF V600x, or EGFR L858R.

### SEQUENCE LISTING

The instant application contains a Sequence Listing which has been submitted electronically in XML format and is hereby incorporated by reference in its entirety. Said XML copy, created on Jan. 31, 2024, is named “51121-088WO2_Sequence_Listing_1_31_24” and is 4,986 bytes in size.

### BACKGROUND

Subject compliance of a dosing regimen and ease of administration of a drug are two important concerns regarding drug development. One solution to these concerns can be a long-acting drug formulation. Long-acting formulations have a wide range of applications such as oral, injectable, transdermal, inhalation, peroral, ocular, pulmonary and topical administration methods. Long-acting injectable formulations can potentially help to improve compliance, reduce dosing frequency, reduce total required dose, reduce toxicity, improve efficacy, and decrease intra-subject variability. There is an unmet need for long-acting formulations of compounds.

### SUMMARY

The present disclosure features compounds and methods useful for treating cancer.

In one aspect, the present disclosure provides a pharmaceutically acceptable composition comprising a long-acting formulation of a compound that reduces the level and/or activity of a protein, or a pharmaceutically acceptable salt thereof, and a pharmaceutically acceptable excipient.

In one aspect, the present disclosure provides a pharmaceutically acceptable composition comprising a long-acting formulation of a compound that reduces the level and/or activity of BRD9, CBP, EP300, estrogen receptor, androgen receptor, IRAK4, IRAK4 and/or IMiD, STAT3, MDM2, BCLXL, BTK, BTK and/or IMiD, TRK, BRM, IKZF1 and/or IKZF3, BRAF V600x, or EGFR L858R, or a pharmaceutically acceptable salt thereof, and a pharmaceutically acceptable excipient.

In an aspect, the disclosure features of a pharmaceutically acceptable composition comprising a long-acting formulation of a compound having the Formula I:

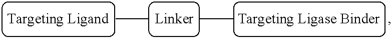

wherein, the Targeting Ligand is a group that is capable of binding to a target protein; the Linker is a group that covalently links the Targeting Ligand to the Targeting Ligase Binder; the Targeting Ligase Binder is a group that is capable of binding to a ligase (e.g., Cereblon E3 Ubiquitin ligase, or VHL E3 ubiquitin ligase binding moiety);
or a pharmaceutically acceptable salt thereof, and a pharmaceutically acceptable excipient.

In some embodiments, the targeting ligase binder is an E3 ligase ligand.

In some embodiments, the targeting ligase binder is an E3 ubiquitin ligase (cereblon) binding moiety, VHL E3 ubiquitin ligase binding moiety, MDM2 (i.e. human double minute 2 or HDM2) E3 ligase binding moiety, or IAP E3 ubiquitin ligase binding moiety. In some embodiments, the targeting ligase binder includes the structure of Formula A:

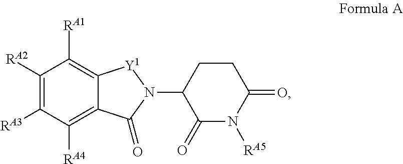

where
each of R^{A1}, R^{A2}, R^{A3}, and R^{A4} is, independently, H, a bond to the linker, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form an optionally substituted C₆-C₁₀ aryl, optionally substituted C₃-C₁₀ carbocyclyl, optionally substituted C₂-C₉ heteroaryl, or C₂-C₉ heterocyclyl;
R^{A5} is H, optionally substituted C₁-C₆ alkyl, or optionally substituted C₁-C₆ heteroalkyl;

Y¹ is

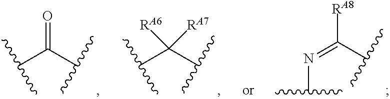

each of R^{A6} and R^{A7} is, independently, H, halogen, or optionally substituted C₁-C₆ alkyl; or R^{A6} and R^{A7}, together with the carbon atom to which each is bound, form an optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
where one of R^{A1}, R^{A2}, R^{A3}, and R^{A4} is a bond to the linker, or a pharmaceutically acceptable salt thereof.

In some embodiments, each R^{A1}, R^{A2}, R^{A3}, and R^{A4} is, independently, a bond to the linker, H, optionally substituted C₁-C₆ alkyl, optionally substituted C₁-C₆ heteroalkyl, hydroxyl, optionally substituted amino; or R^{A1} and R^{A2}, R^{A2} and R^{A3}, or R^{A3} and R^{A4}, together with the carbon atoms to which each is attached, combine to form an optionally substituted C₂-C₉ heterocyclyl.

In some embodiments, the structure of Formula A is

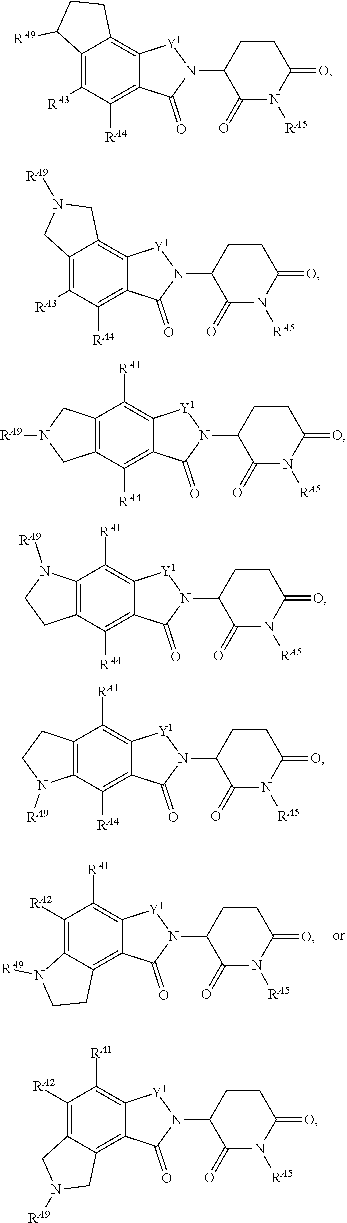

where R^{A9} is H, optionally substituted C₁-C₆ alkyl, or optionally substituted C₁-C₆ heteroalkyl.

In some embodiments, R^{A1} is a bond to the linker. In some embodiments, R^{A2} is a bond to the linker. In some embodiments, R^{A3} is a bond to the linker. In some embodiments, R^{A4} is a bond to the linker. In some embodiments, R^{A5} is a bond to the linker.

In some embodiments, R^{A5} is H or optionally substituted C₁-C₆ alkyl.

In some embodiments, R^{A5} is H or

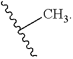

In some embodiments, R^{A5} is H. In some embodiments, R^{A5} is

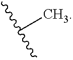

In some embodiments, Y¹ is

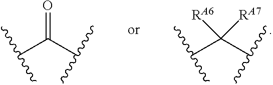

In some embodiments, Y¹ is

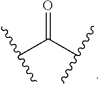 [SMILES: *C(*)=O]

In some embodiments, Y¹ is

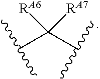

In some embodiments, each of R^{A6} and R^{A7} is, independently, H, F,

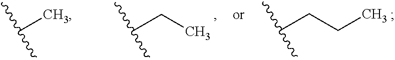

or R^{A6} and R^{A7}, together with the carbon atom to which each is bound, form

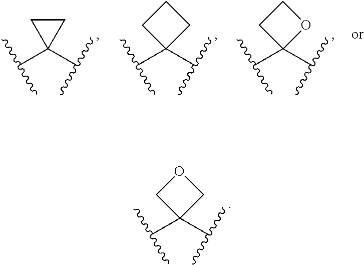 [SMILES: *C1(*)CC1 ; *C1(*)CCC1 ; *C1(*)CCO1 ; *C1(*)COC1]

In some embodiments, Y¹ is

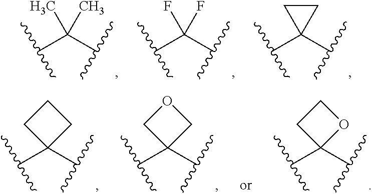

In some embodiments, the structure of Formula A has the structure of Formula A1:

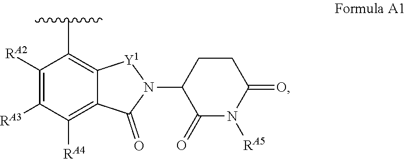

or a pharmaceutically acceptable salt thereof.

In some embodiments, the structure of Formula A has the structure of Formula A2:

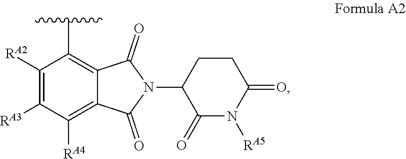

or a pharmaceutically acceptable salt thereof.

In some embodiments, the structure of Formula A has the structure of Formula A3:

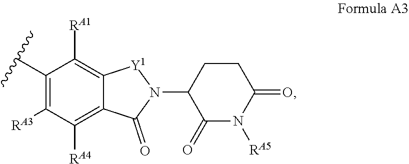

or a pharmaceutically acceptable salt thereof.

In some embodiments, the structure of Formula A has the structure of Formula A4:

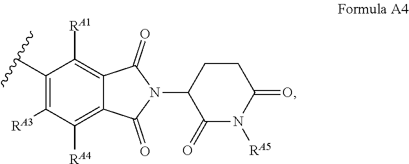

or a pharmaceutically acceptable salt thereof.

In some embodiments, the structure of Formula A is

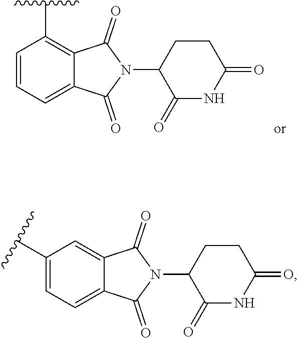 [SMILES: *c1cccc2c1C(=O)N(C1CCC(=O)NC1=O)C2=O ; *c1ccc2c(c1)C(=O)N(C1CCC(=O)NC1=O)C2=O]

or is a derivative or an analog thereof.

In some embodiments, the targeting ligase binder includes the structure of Formula B:

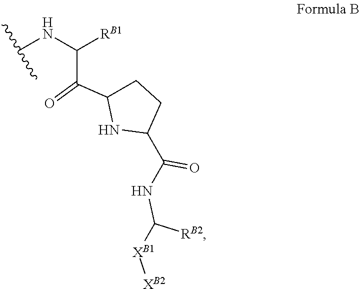

where
each of R^{B1}, and R^{B2} is, independently, H, a bond to the linker, halogen, optionally substituted C₁-C₆ alkyl, optionally substituted C₁-C₆ heteroalkyl, or optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with CH₃, OH, OCH₃ and/or F.
each X^{B1} is a bond to the linker, halogen, optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with one or more groups of R^{B3};
where R^{B3} is optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl,
each X^{B2} is a bond to the linker, halogen, —CN, optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with one or more groups of R^{B4}; where R^{B4} is optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with one or more groups of R^{B5};
where R^{B5} is CH₃, OH, OCH₃ and/or F.

In some embodiments, R^{B1} is

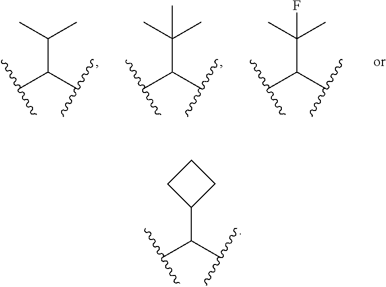

In some embodiments, R^{B2} is CH₃ CH₂OH, or CH₂F.

In some embodiments, X^{B1} is

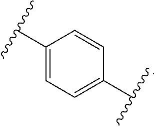 [SMILES: *c1ccc(*)cc1]

In some embodiments, X^{B2} is

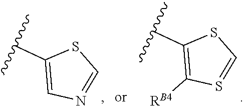

In some embodiments, X^{B2} is CN.

In some embodiments, X^{B2} is

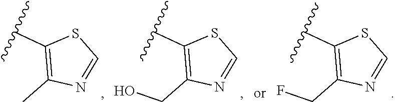

In some embodiments, the targeting ligase binder has the structure:

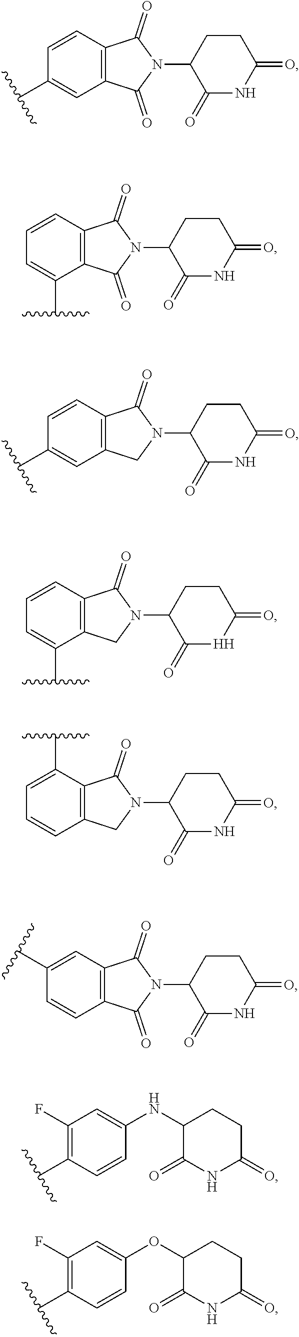

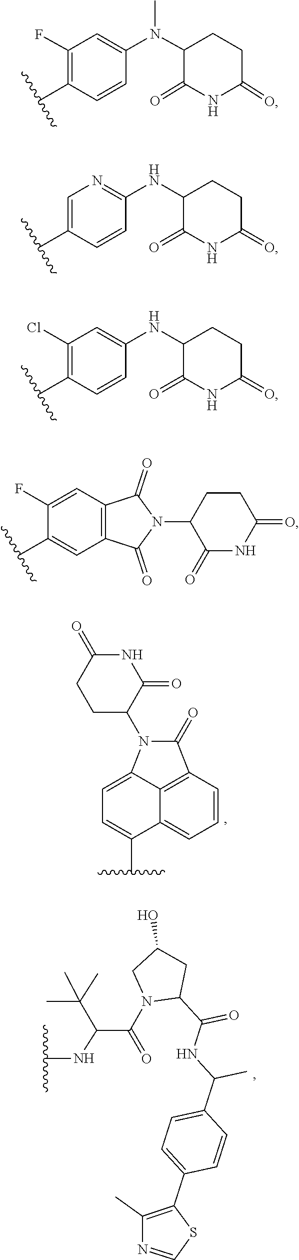 [SMILES: *c1ccc(N(C)C2CCC(=O)NC2=O)cc1F ; *c1ccc(NC2CCC(=O)NC2=O)nc1 ; *c1ccc(NC2CCC(=O)NC2=O)cc1Cl ; *c1cc2c(cc1F)C(=O)N(C1CCC(=O)NC1=O)C2=O ; *c1ccc2c3c(cccc13)C(=O)N2C1CCC(=O)NC1=O ; *NC(C(=O)N1C[C@H](O)CC1C(=O)NC(C)c1ccc(-c2scnc2C)cc1)C(C)(C)C]

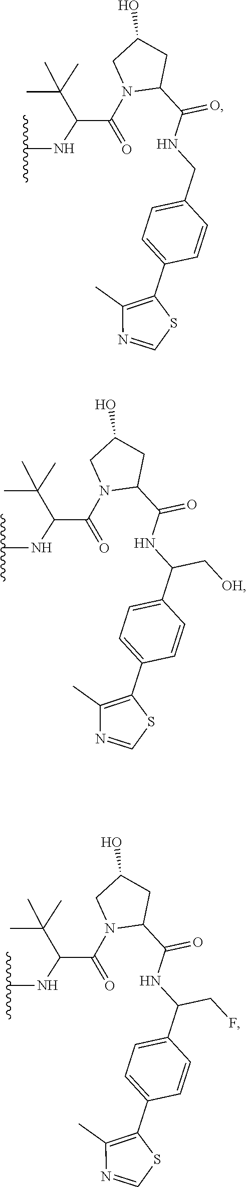 [SMILES: *NC(C(=O)N1C[C@H](O)CC1C(=O)NCc1ccc(-c2scnc2C)cc1)C(C)(C)C ; *NC(C(=O)N1C[C@H](O)CC1C(=O)NC(CO)c1ccc(-c2scnc2C)cc1)C(C)(C)C ; *NC(C(=O)N1C[C@H](O)CC1C(=O)NC(CF)c1ccc(-c2scnc2C)cc1)C(C)(C)C]

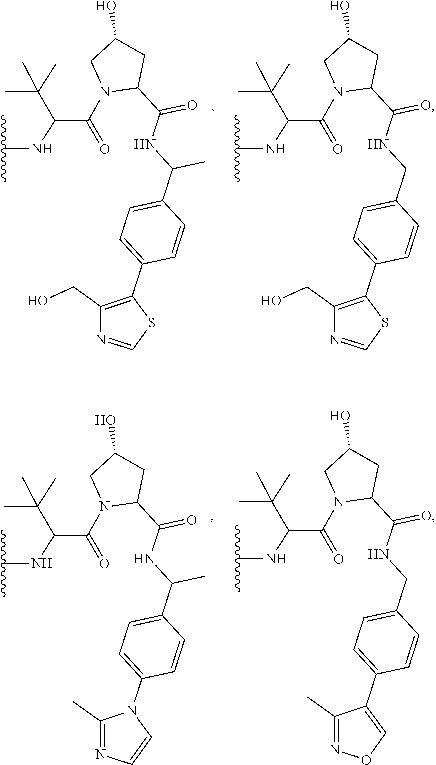

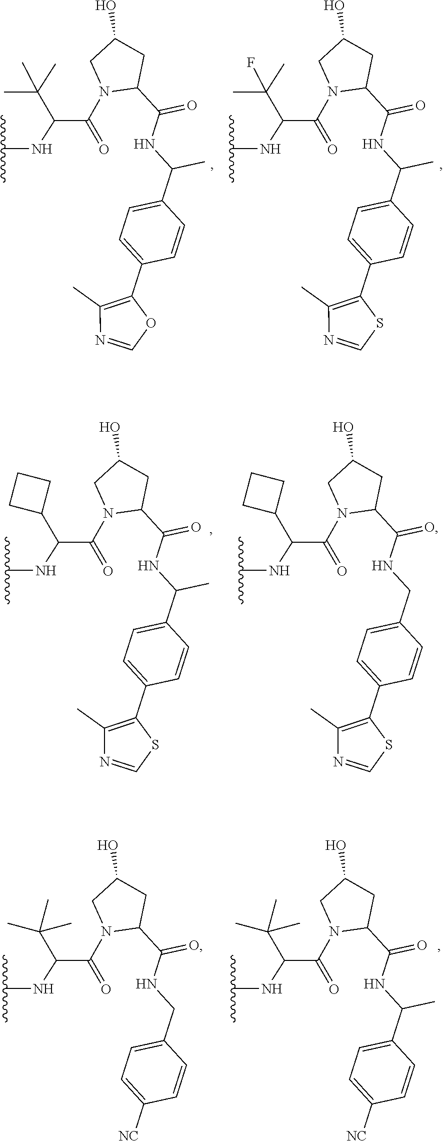

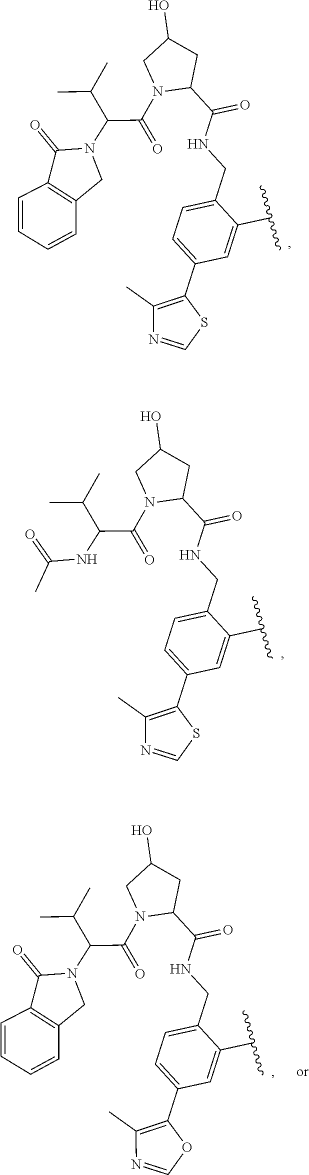 [SMILES: *c1cc(-c2scnc2C)ccc1CNC(=O)C1CC(O)CN1C(=O)C(C(C)C)N1Cc2ccccc2C1=O ; *c1cc(-c2scnc2C)ccc1CNC(=O)C1CC(O)CN1C(=O)C(NC(C)=O)C(C)C ; *c1cc(-c2ocnc2C)ccc1CNC(=O)C1CC(O)CN1C(=O)C(C(C)C)N1Cc2ccccc2C1=O]

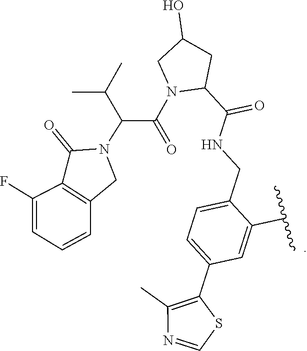 [SMILES: *c1cc(-c2scnc2C)ccc1CNC(=O)C1CC(O)CN1C(=O)C(C(C)C)N1Cc2cccc(F)c2C1=O]

In some embodiments, the targeting ligase binder has the structure:

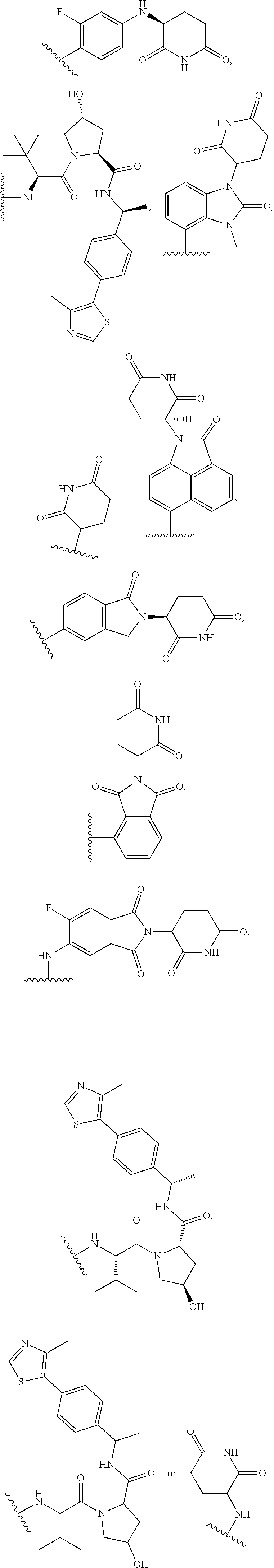

In some embodiments, the targeting ligand has the structure:

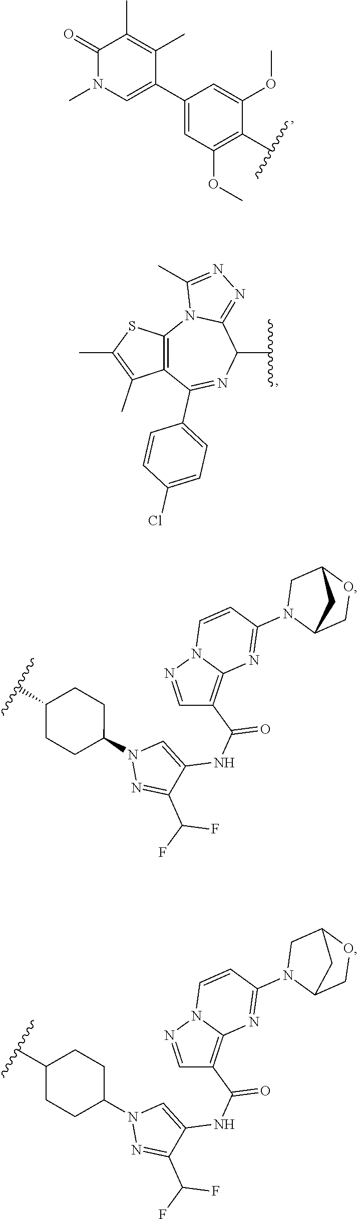

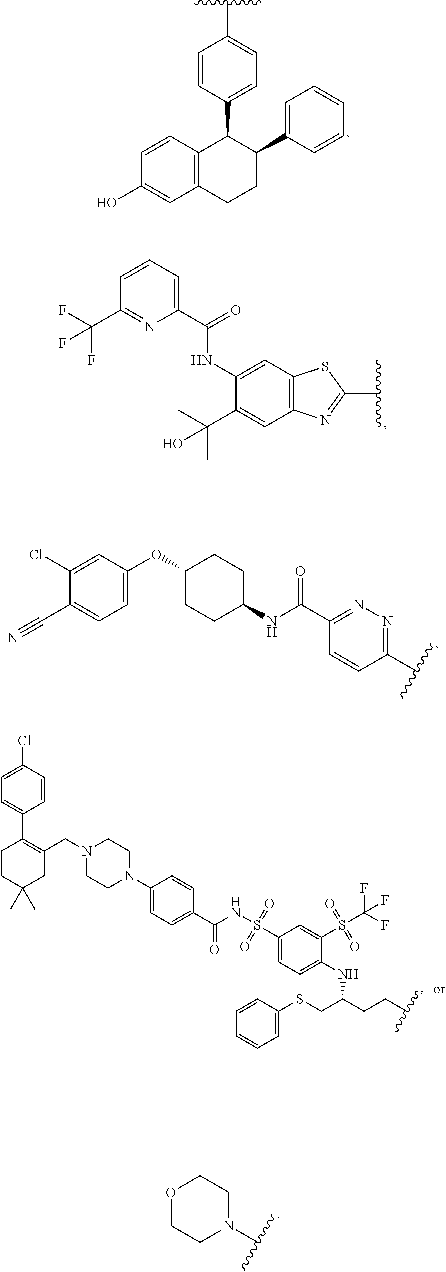 [SMILES: *c1ccc([C@@H]2c3ccc(O)cc3CC[C@@H]2c2ccccc2)cc1 ; *c1nc2cc(C(C)(C)O)c(NC(=O)c3cccc(C(F)(F)F)n3)cc2s1 ; *c1ccc(C(=O)N[C@H]2CC[C@H](Oc3ccc(C#N)c(Cl)c3)CC2)nn1 ; *CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F ; *N1CCOCC1]

In some embodiments, the targeting ligase binder comprises the structure of Formula IVa:

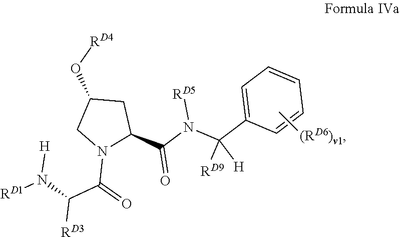

wherein
R^{D1} is H, A², —C(O)-A², optionally substituted C₁-C₆ alkyl, or optionally substituted C₁-C₆ heteroalkyl;
R^{D3} is A², —C(O)-A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl, optionally substituted C₃-C₁₀ carbocyclyl, or optionally substituted C₁-C₆ alkyl wherein the C₁-C₆ alkyl, C₁-C₆ heteroalkyl, C₃-C₁₀ carbocyclyl, C₆-C₁₀ aryl, C₁-C₆ alkyl, C₃-C₁₀ carbocyclyl, or C₆-C₁₀ aryl is substituted with A² and/or one of more groups of R^{J2};
R^{D4} is H, optionally substituted C₁-C₆ alkyl, optionally substituted C₃-C₁₀ carbocyclyl, or optionally substituted C₆₋₁₀ aryl;
R^{D5} is H, optionally substituted C₁-C₆ alkyl, or optionally substituted C₁-C₆ heteroalkyl;
v1 is 0, 1, 2, 3, or 4;
each R^{D6} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂₋₆ alkenyl, optionally substituted C₂-C₆ heteroalkenyl, optionally substituted C₂-C₆ alkynyl, hydroxy, thiol,
R^{D9} is H, or optionally substituted C₁-C₆ alkyl;
each R^{J2} is, independently, hydrogen, C₁-C₆ alkyl, C₃-C₁₂ carbocyclyl, and C₃-C₁₂ heterocyclyl, wherein each C₁-C₆ alkyl, C₃-C₁₂ carbocyclyl, and C₃-C₁₂ heterocyclyl is optionally substituted with one or more groups independently selected from amino, hydroxyl, C₁-C₆ alkoxy, C₃-C₁₂ carbocyclyl, C₃-C₁₂ heterocyclyl, or C₁-C₆ alkyl that is optionally substituted with A² and/or one or more groups independently selected from oxo and halo; and
wherein either R^{D1} or R^{D3} comprises A² or —C(O)-A²;
wherein A² is a bond to the linker;
or a pharmaceutically acceptable salt thereof.

In some embodiments, R^{D6} is optionally substituted C₂-C₉ heteroaryl.

In some embodiments, R^{D6} is:

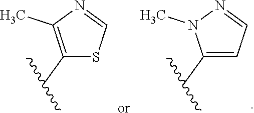

In some embodiments, the structure of Formula IVa is

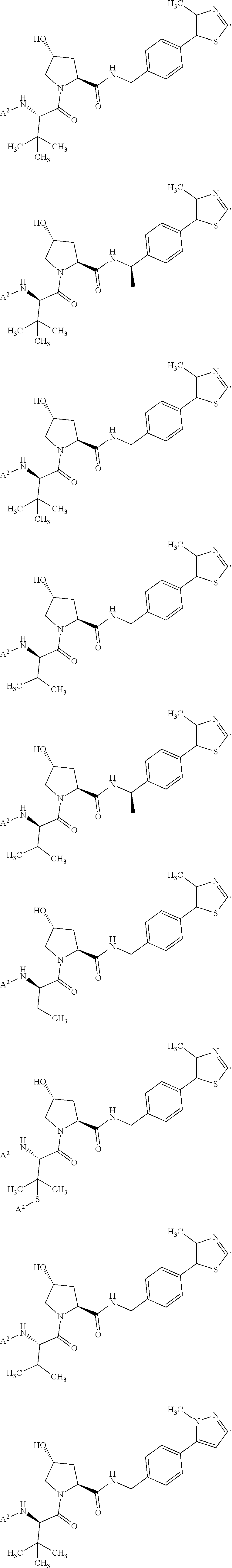

or a derivative or analog thereof.

In some embodiments, R^{D6} is halogen or optionally substituted C₂-C₆ alkynyl.

In some embodiments, R^{D6} is optionally substituted C₁-C₆ heteroalkyl.

In some embodiments, optionally substituted C₁-C₆ heteroalkyl is methoxy.

In some embodiments, the structure of Formula IVa is

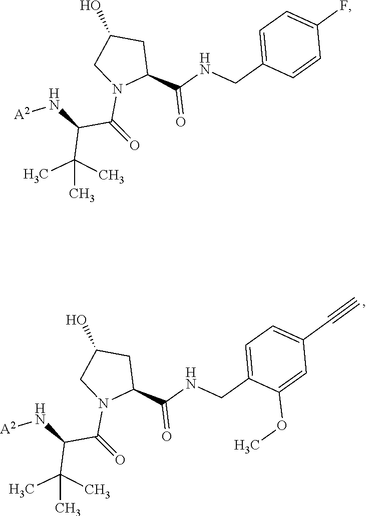

or a derivative or analog thereof.

In some embodiments, the targeting ligase binder comprises the structure of Formula IV:

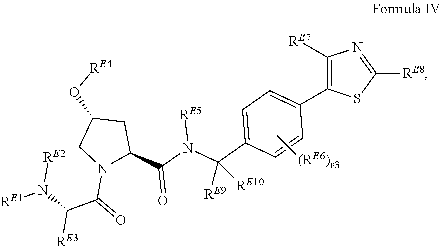

wherein
R^{E1} is H, A², optionally substituted C₁-C₆ alkyl, or optionally substituted C₁-C₆ heteroalkyl;
R^{E2} is H, optionally substituted C₁-C₆ alkyl, or optionally substituted C₁-C₆ heteroalkyl;
R^{E3} is A², optionally substituted C₁-C₆ alkyl, optionally substituted C₁-C₆ heteroalkyl, optionally substituted C₃-C₁₀ carbocyclyl, or optionally substituted C₆-C₁₀ aryl;
R^{E4} is H, optionally substituted C₁-C₆ alkyl, optionally substituted C₃-C₁₀ carbocyclyl, or optionally substituted C₆-C₁₀ aryl;
R^{E5} is H, optionally substituted C₁-C₆ alkyl, or optionally substituted C₁-C₆ heteroalkyl;
v3 is 0, 1, 2, 3, or 4;
each R^{E6} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R^{E7} and R^{E8} is, independently, H, halogen, optionally substituted C₁-C₆ alkyl, or optionally substituted C₆₋₁₀ aryl,
R^{E9} and R^{BE0} are, independently, H, or optionally substituted C₁-C₆ alkyl,
wherein one of R^{E1} and R^{E3} is A²;
wherein A² is a bond to the linker;
or a pharmaceutically acceptable salt thereof.

In some embodiments, the structure of Formula IV is

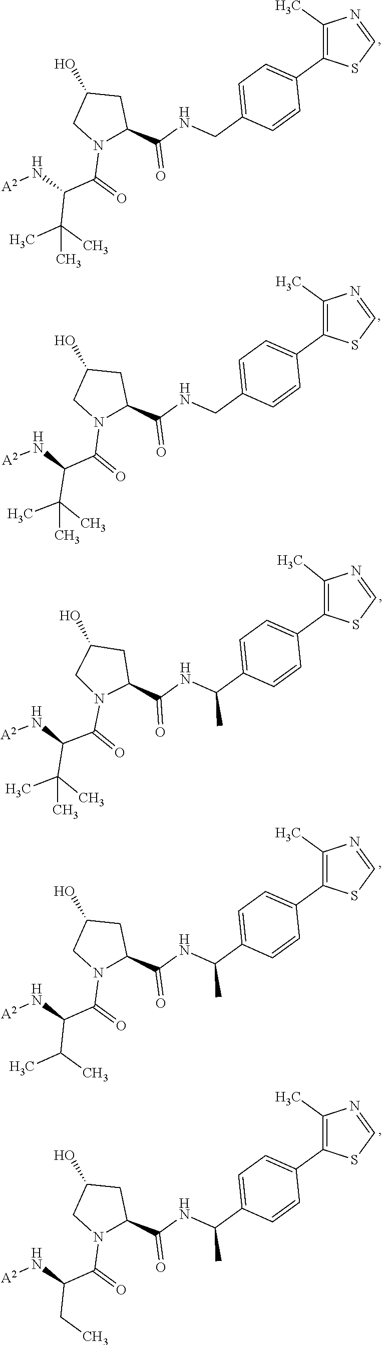

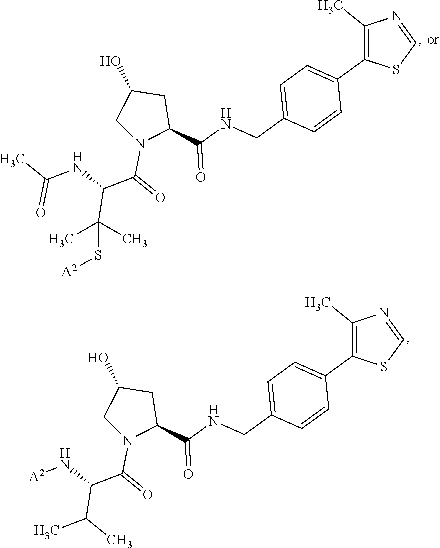

or derivative or analog thereof.

In some embodiments, the targeting ligase binder has the structure:

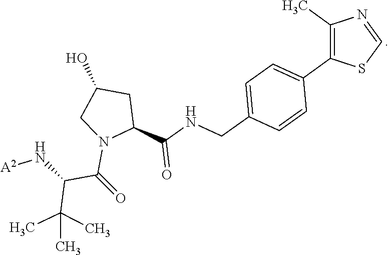

In some embodiments, the targeting ligase binder has the structure:

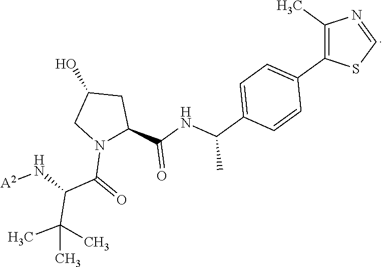

In some embodiments, the targeting ligase binder comprises the structure of Formula V:

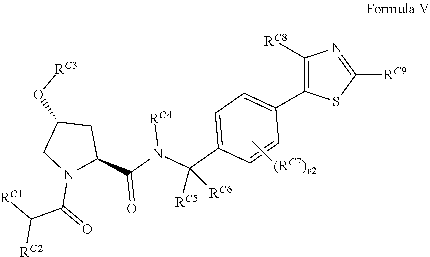

R^{C1} is optionally substituted C₁-C₆ alkyl;
R^{C2} is A², or optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₂-C₉ heteroaryl, wherein each C₁-C₆ alkyl, C₃-C₁₀ carbocyclyl, C₁-C₆ heteroalkyl, C₆-C₁₀ aryl, or C₂-C₉ heteroaryl is optionally substituted with A² and/or one or more groups R^{J};
R_{C3} is H, optionally substituted C₁-C₆ alkyl, optionally substituted C₃-C₁₀ carbocyclyl, or optionally substituted C₆-C₁₀ aryl;
R^{C4} is H, optionally substituted C₁-C₆ alkyl, or optionally substituted C₁-C₆ heteroalkyl;
v2 is 0, 1, 2, 3, or 4;
each of R^{C5} and R^{C6} is, independently, H, or optionally substituted C₁-C₆ alkyl each R^{C7} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R^{C8} and R^{C9} is, independently, H, halogen, optionally substituted C₁-C₆ alkyl, or optionally substituted C₆₋₁₀ aryl,
each R^{J} is, independently, hydrogen, C₁-C₆ alkyl, C₃-C₆ carbocyclyl, and C₂-C₆ heterocyclyl, wherein each C₁-C₆ alkyl, C₃-C₆ carbocyclyl, and C₂-C₆ heterocyclyl is optionally substituted with one or more groups independently selected from amino, hydroxyl, C₁-C₆ alkoxy, C₃-C₆ carbocyclyl, C₂-C₆ heterocyclyl, or C₁-C₆ alkyl that is optionally substituted with A² and/or one or more groups independently selected from oxo and halo;
wherein A² is a bond to the linker;
or a pharmaceutically acceptable salt thereof.

In some embodiments, the targeting ligase binder has the structure:

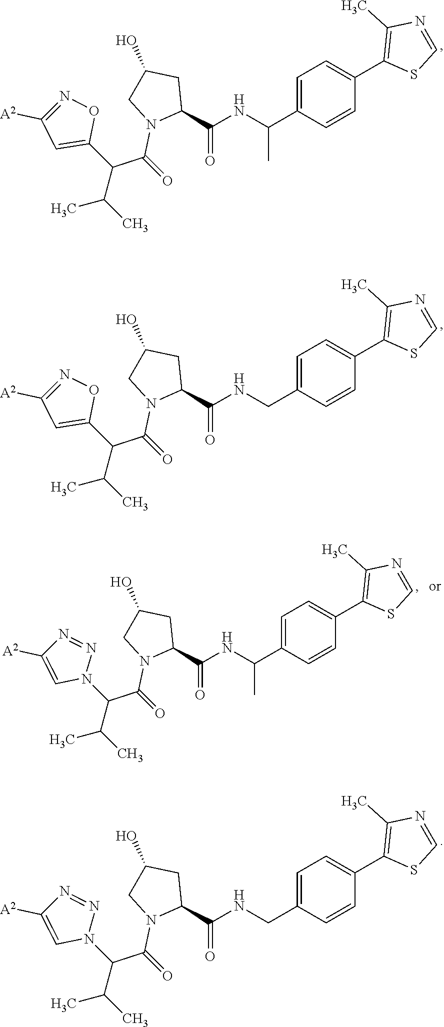

In some embodiments, the targeting ligase binder comprises the structure of Formula IVb:

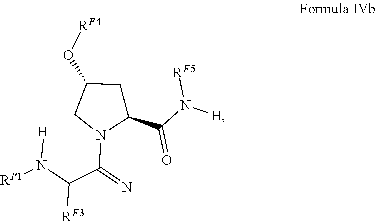

wherein
R^{F1} is H, A², —C(O)-A², optionally substituted C₁-C₆ alkyl, or optionally substituted C₁-C₆ heteroalkyl;
R^{F3} is A², —C(O)-A², optionally substituted C₁-C₆ alkyl, optionally substituted C₁-C₆ heteroalkyl, optionally substituted C₃-C₁₀ carbocyclyl, or optionally substituted C₆-C₁₀ aryl;
R^{F4} is H, optionally substituted C₁-C₆ alkyl, optionally substituted C₃-C₁₀ carbocyclyl, optionally substituted C₆-C₁₀ aryl;
R^{F5} is H, optionally substituted C₁₋₆ alkyl, or optionally substituted C₁-C₆ heteroalkyl or optionally substituted arylalkyl;
wherein one of R^{F1} and R^{F3} is A² or —C(O)-A²;
wherein A² is a bond to the linker;
or a pharmaceutically acceptable salt thereof.

In some embodiments, the structure of Formula IVb is

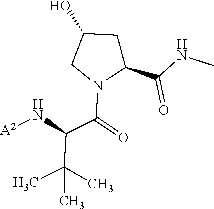

or a derivative or analog thereof.

In some embodiments, the linker has the structure:

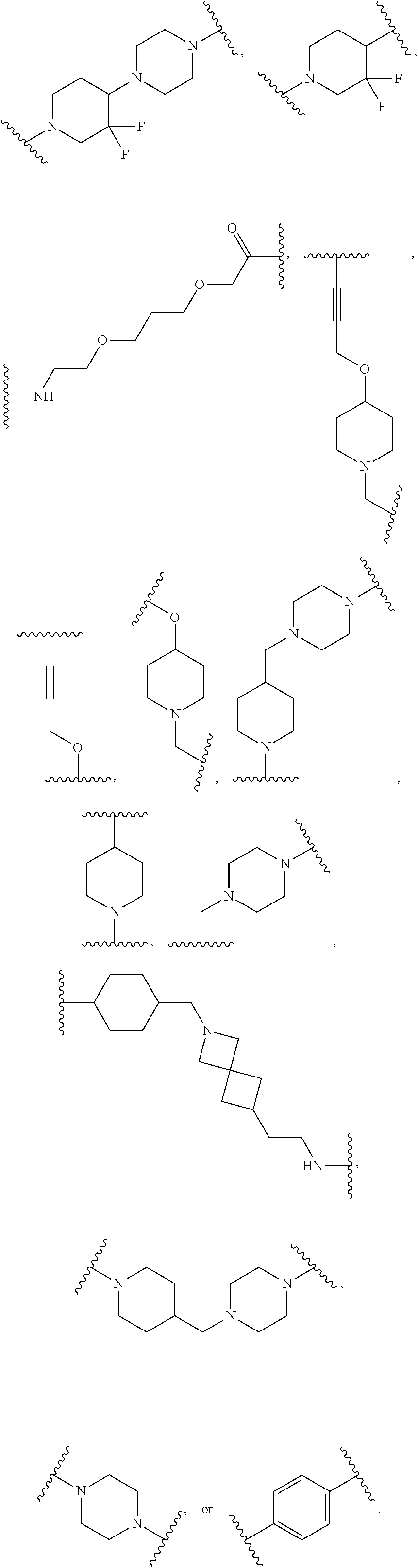

In some embodiments, the linker has the structure: —N(R)—(CH2)ₘ-O(CH2)ₙ-O(CH2)ₒ-O(CH2)ₚ-O(CH2)_{q}-O(CH2)ᵣ-OCH2-, —O—(CH2)ₘ-O(CH2)ₙ-O(CH2)ₒ-O(CH2)ₚ-O(CH2)_{q}-O(CH2)ᵣ-OCH2-, —O—(CH2)ₘ-O(CH2)ₙ-O(CH2)ₒ-O(CH2)ₚ-O(CH2)_{q}-O(CH2)ᵣ-O—, —N(R)—(CH2)ₘ-O(CH2)ₙ-O(CH2)ₒ-O(CH2)ₚ-O(CH2)_{q}-O(CH2)ᵣ-O—, —(CH2)ₘ-O(CH2)ₙ-O(CH2)ₒ-O(CH2)ₚ—O(CH2)_{q}-O(CH2)ᵣ-O—, —(CH2)ₘ-O(CH2)ₙ-O(CH2)ₒ-O(CH2)ₚ-O(CH2)_{q}-O(CH2)ᵣ-OCH2-,

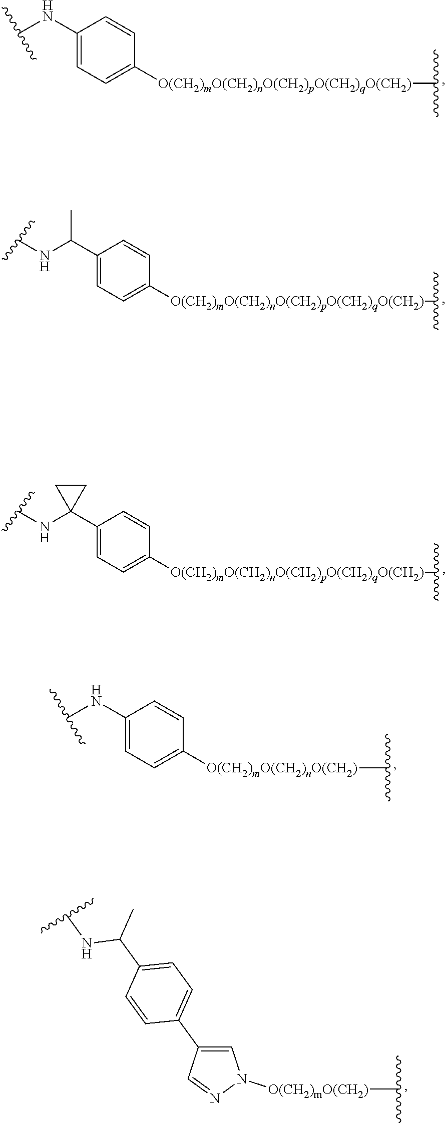

C(O)—(CH2)ₘ-O(CH2)ₙ-O(CH2)ₒ-O(CH2)ₚ-O(CH2)_{q}-O(CH2)ᵣ-OCH2-C(O), C(O)—(CH2)ₘ-O(CH2)ₙ-O(CH2)ₒ-O(CH2)ₚ-O(CH2)_{q}-OCH2-C(O), C(O)—(CH2)ₘ-O(CH2)ₙ-O(CH2)ₒ-O(CH2)ₚ-OCH2-C(O), C(O)—(CH2)ₘ-O(CH2)ₙ-O(CH2)ₒ-O(CH2)ᵣ-OCH2-C(O), or C(O)—(CH2)ₘ-OCH2-C(O);
wherein m, n, o, p, q and r are 1,2,3, or 4.

In some embodiments, the linker is an optionally substituted C₁-C₁₀ alkyl, C₁-C₁₀ heteroalkyl, C₁-C₁₀ alkynyl, 3-12 membered carbocycle, or 3-12 membered heterocycle, wherein each C₁-C₁₀ alkyl, C₁-C₁₀ alkynyl, 3-12 membered carbocycle, or 3-12 membered heterocycle of L² is optionally substituted with LBM, and/or one or more groups of R^{A10}.

Wherein R^{A10} is C₁-C₁₀ alkyl, C₁-C₁₀ alkynyl, 3-12 membered carbocycle, or 3-12 membered heterocycle is independently optionally substituted with LBM, and/or one or more of the substituent groups selected from CH₃, OH, OCH₃ and F.

In one embodiment of any of the compounds above, the compound includes only one linker and/or only one linkage group to a linker.

In some embodiments, the targeting ligand binds to BRD9, CBP, EP300, estrogen receptor, androgen receptor, IRAK4, IRAK4 and/or IMiD, STAT3, MDM2, BCLXL, BTK, BTK and/or IMiD, TRK, BRM, IKZF1 and/or IKZF3, BRAF V600x, or EGFR L858R.

In one aspect, the present disclosure provides a pharmaceutically acceptable composition comprising a long-acting formulation of a compound of the present disclosure that reduces the level and/or activity of BRD9, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of CBP, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of EP300, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of estrogen receptor, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of androgen receptor, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of IRAK4, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of IRAK4 and/or IMiD, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of STAT3, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of MDM2, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of BCLXL, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of BTK, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of BTK and/or MiD, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of TRK, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of BRM, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of IKZF1 and/or IKZF3, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of BRAF V600x, or a pharmaceutically acceptable salt thereof, and a pharmaceutically acceptable excipient.

In one aspect, the present disclosure provides a pharmaceutically acceptable composition comprising a long-acting formulation of a compound of the present disclosure that reduces the level and/or activity of EGFR L858R, or a pharmaceutically acceptable salt thereof, and a pharmaceutically acceptable excipient.

In some embodiments of any of the aspects described herein, the compound is:

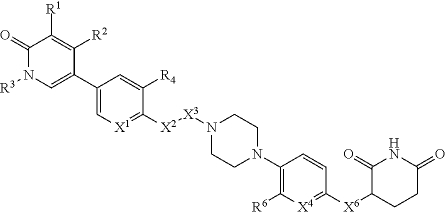

R¹ is NH₂, or CH₃;
R² is H, or CH₃;
R³ is H, CH₃ or OCH₃;
R⁴ is H, CH₃, OCH₃, CF₂H, or CF₃;
R5 is H, OH, CH₃, CFH2, F, Cl, Br, or I;
X¹ is CH, N, COCH₃, or CCF₃;
X² is CH₂, C(O), or O;
X³ is heterocycle optionally substituted with 1, 2, or 3 groups independently selected from R^{X};
X⁴ is N, CH, or CCH₃;
X⁵ is NH, NCH₃, CH₂, O, and S;

In some embodiments, X³ is

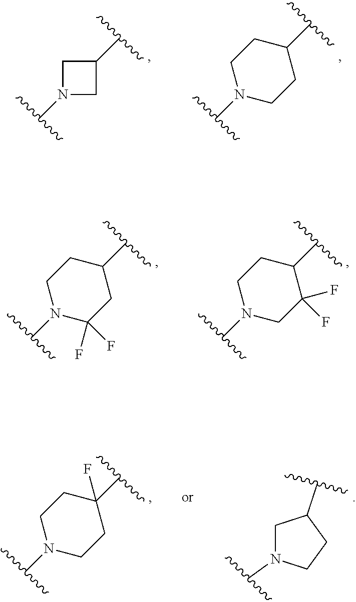 [SMILES: *C1CN(*)C1 ; *C1CCN(*)CC1 ; *C1CCN(*)C(F)(F)C1 ; *C1CCN(*)CC1(F)F ; *N1CCC(*)(F)CC1 ; *C1CCN(*)C1]

In some embodiments of any of the aspects described herein, the compound is:

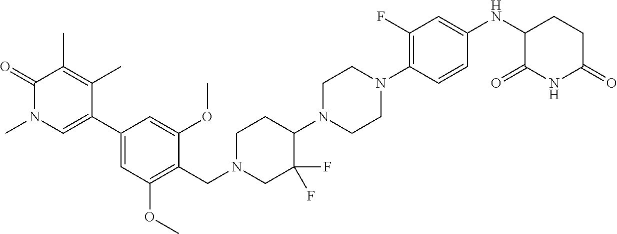 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1]

or a pharmaceutically acceptable salt thereof.

In some embodiments of any of the aspects described herein, the compound is:

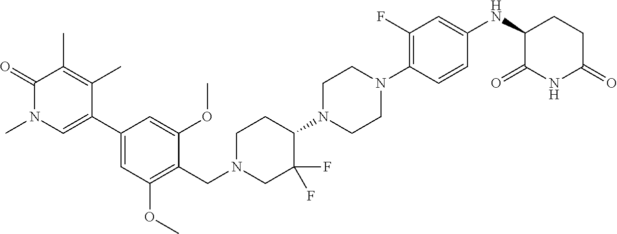 [SMILES: COc1cc(-c2cn(C)c(=O)c(C)c2C)cc(OC)c1CN1CC[C@H](N2CCN(c3ccc(N[C@H]4CCC(=O)NC4=O)cc3F)CC2)C(F)(F)C1]

or a pharmaceutically acceptable salt thereof.

In some embodiments of any of the aspects described herein, the compound is:

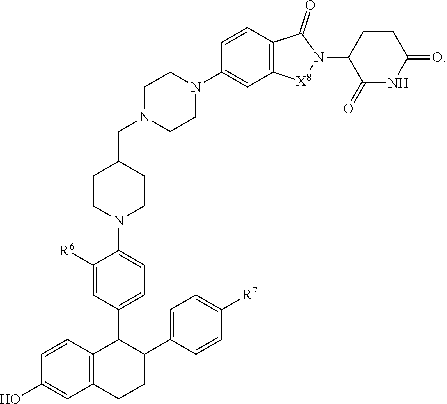

R⁶ is H, F or CH₃,
R⁷ is H, CH₃, OCH₃, or OH;
X⁶ is CH₂, or C(O).

In some embodiments of any of the aspects described herein, the compound is:

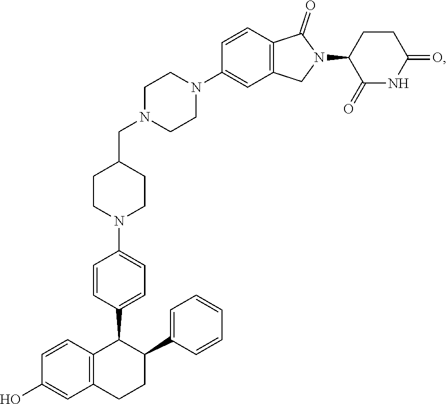 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof.

In some embodiments of any of the aspects described herein, the compound is:

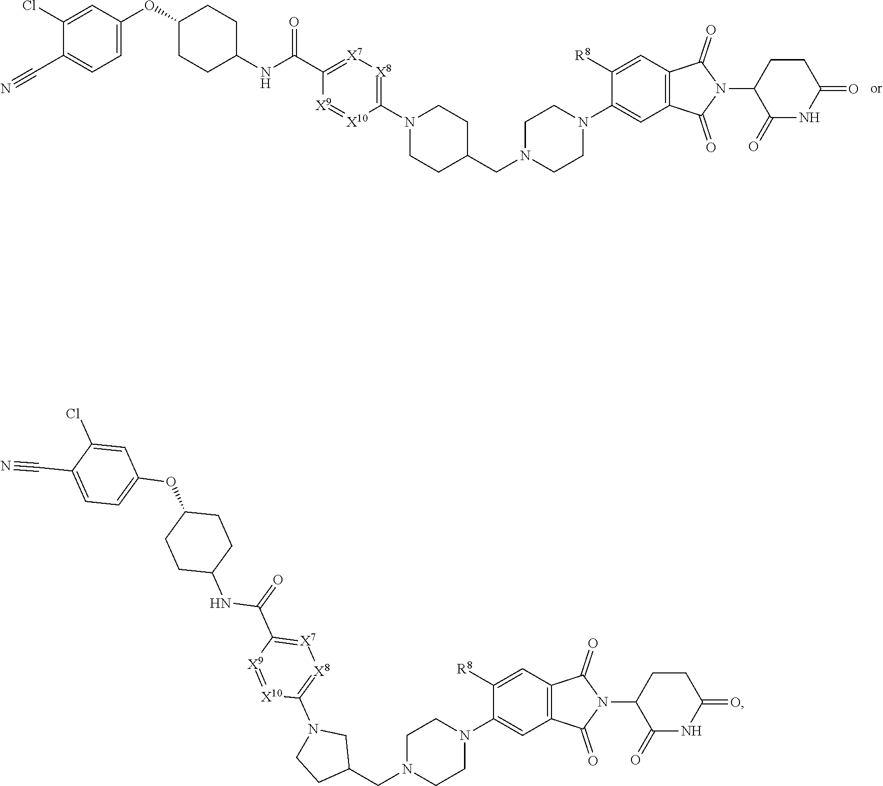

R⁸ is H or F;
X⁷ is CH₂ or N;
X⁸ is CH₂ or N;
X⁹ is CH₂ or N;
X¹⁰ is CH₂ or N.

In some embodiments of any of the aspects described herein, the compound is:

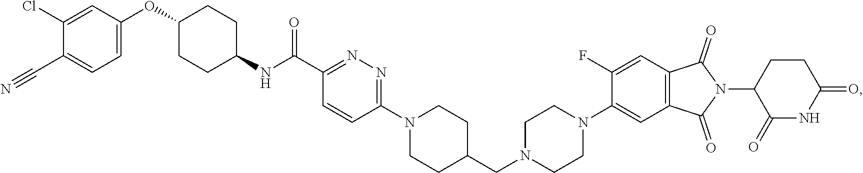 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof.

In some embodiments of any of the aspects described herein, the compound is:

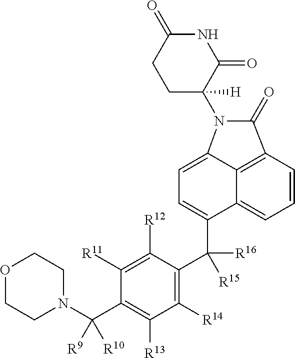

R⁹ is H, or CH₃;
R¹⁰ is H, or CH₃;
R¹¹ is H, or CH₃;
R¹² is H, or CH₃;
R¹³ is H, or CH₃;
R¹⁴ is H, or CH₃;
R¹⁵ is H, or CH₃;
R¹⁶ is H, or CH₃.

In some embodiments of any of the aspects described herein, the compound is:

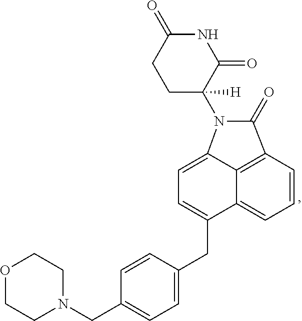 [SMILES: O=C1CC[C@H](N2C(=O)c3cccc4c(Cc5ccc(CN6CCOCC6)cc5)ccc2c34)C(=O)N1]

or a pharmaceutically acceptable salt thereof.

In some embodiments of any of the aspects described herein, the compound is:

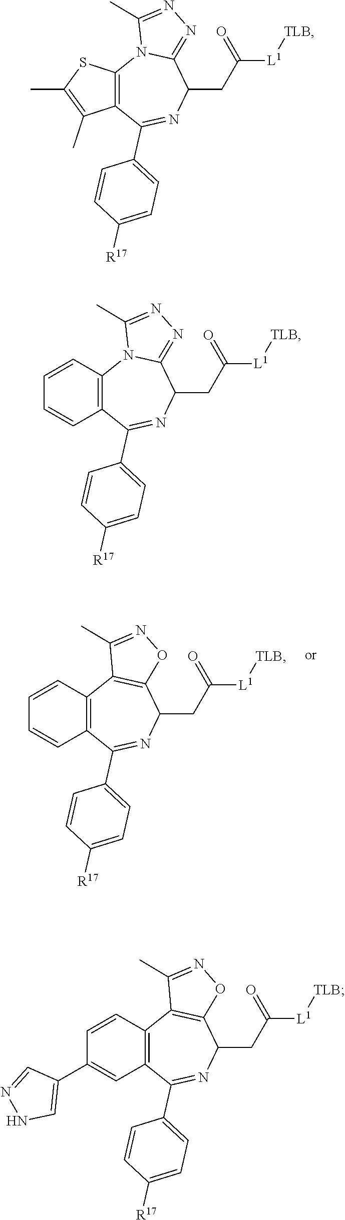

R¹⁷ is H, Cl, F, Br, CN, CH₃, OCH₃, or C(O)CH₃;

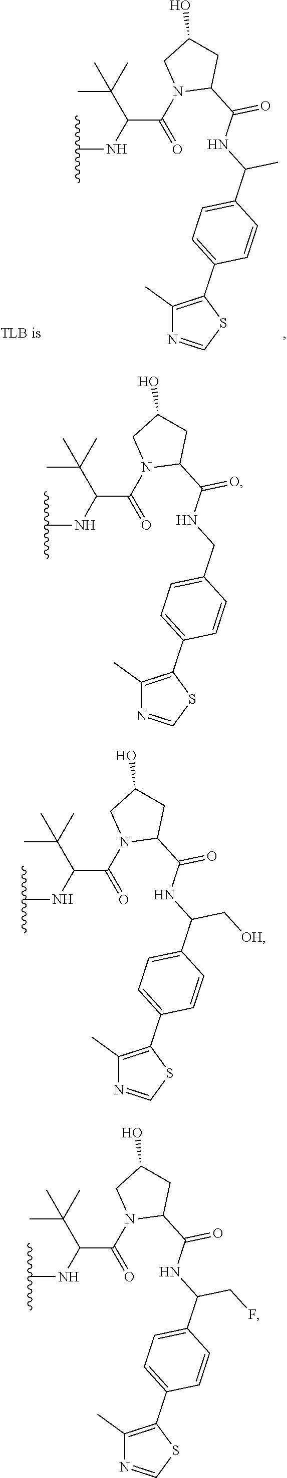

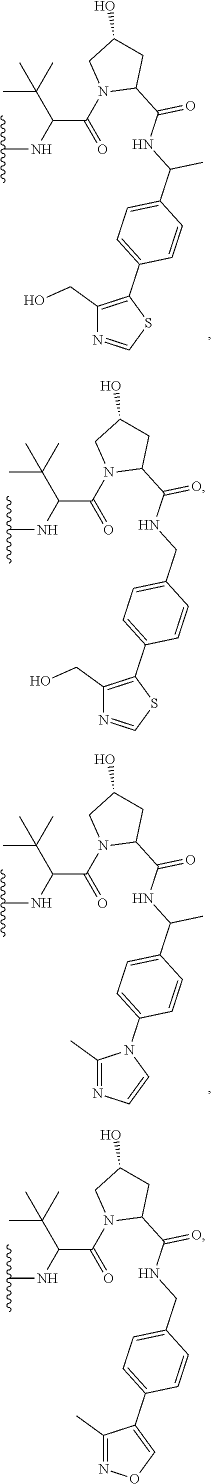

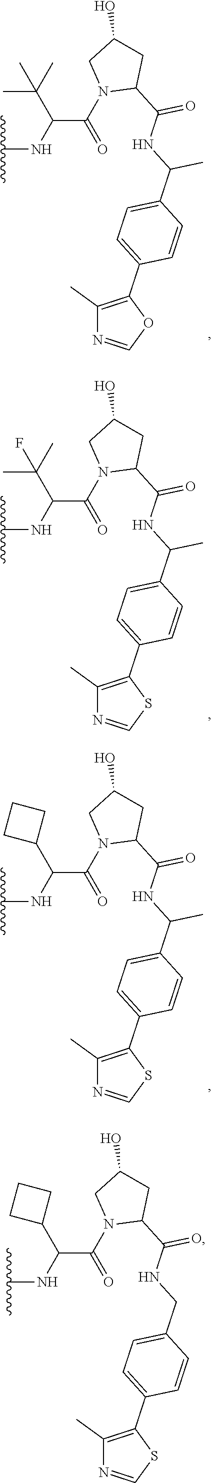

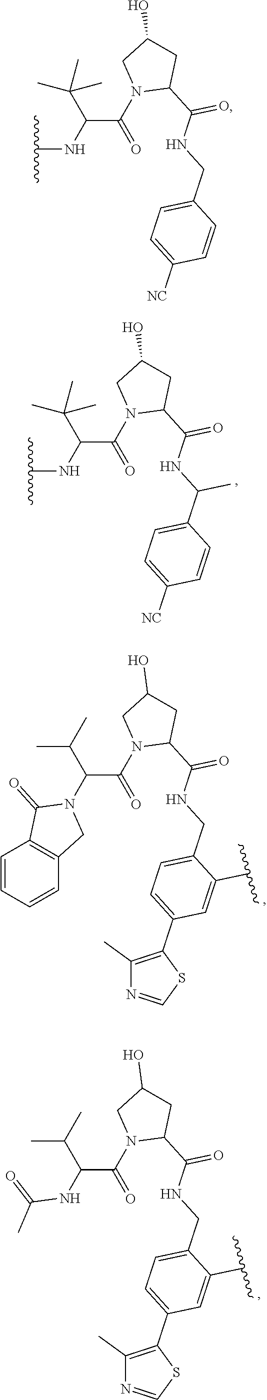

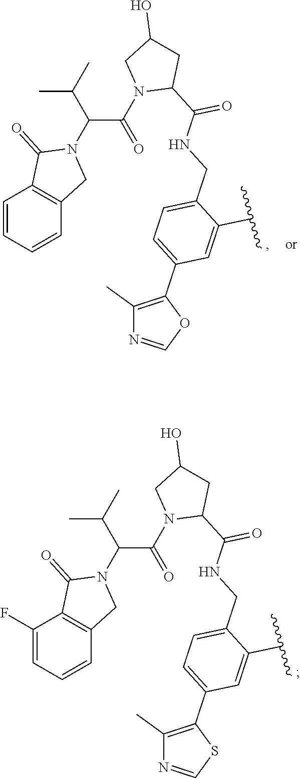 [SMILES: *c1cc(-c2ocnc2C)ccc1CNC(=O)C1CC(O)CN1C(=O)C(C(C)C)N1Cc2ccccc2C1=O ; *c1cc(-c2scnc2C)ccc1CNC(=O)C1CC(O)CN1C(=O)C(C(C)C)N1Cc2cccc(F)c2C1=O]

L¹ is —N(R)—(CH2)ₘ-O(CH2)ₙ-O(CH2)ₒ-O(CH2)ₚ-O(CH2)_{q}-O(CH2)ᵣ, OCH2-, —O—(CH2)ₘ-O(CH2)ₙ-O(CH2)ₒ-O(CH2)ₚ-O(CH2)_{q}-O(CH2)ᵣ-OCH2-, —O—(CH2)ₘ—O(CH2)ₙ—O(CH2)ₒ-O(CH2)ₚ-O(CH2)_{q}—O(CH2)ᵣ-O—, —N(R)—(CH2)ₘ—O(CH2)ₙ—O(CH2)ₒ-O(CH2)ₚ-O(CH2)_{q}-O(CH2)ᵣ-O—, (CH2)ₘ-O(CH2)ₙ-O(CH2)ₒ-O(CH2)ₚ-O(CH2)_{q}-O(CH2)ᵣ-O—, (CH2)ₘ-O(CH2)ₙ-O(CH2)ₒ-O(CH2)ₚ-O(CH2)_{q}-O(CH2)ᵣ-OCH2-,

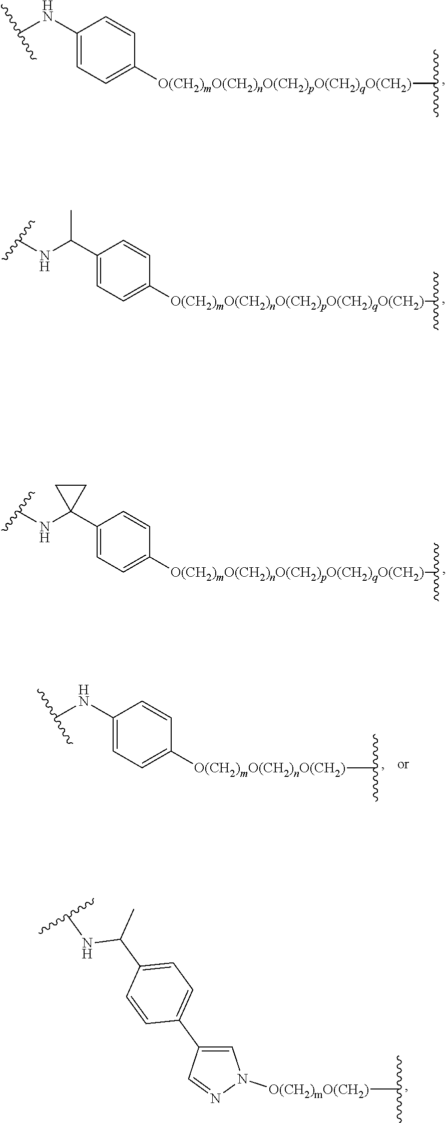

Wherein m, n, o, p, q and r are 1,2,3, or 4.

In some embodiments of any of the aspects described herein, the compound is:

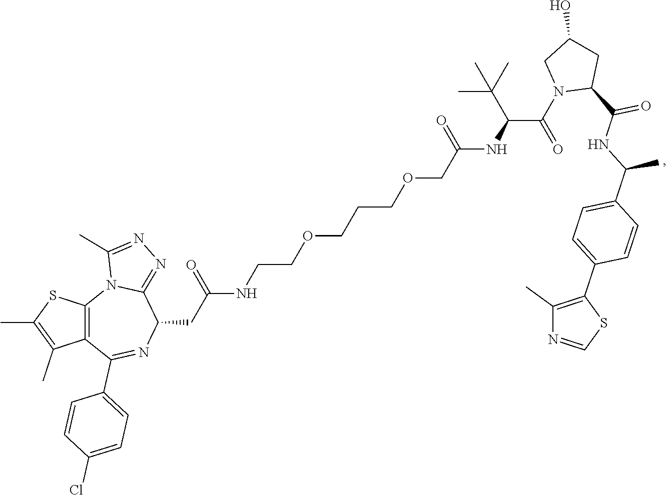 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1]

or a pharmaceutically acceptable salt thereof.

In some embodiments of any of the aspects described herein, the compound is:

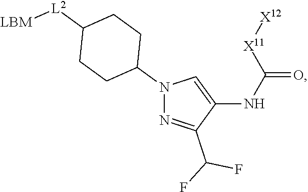

Wherein, LBM is

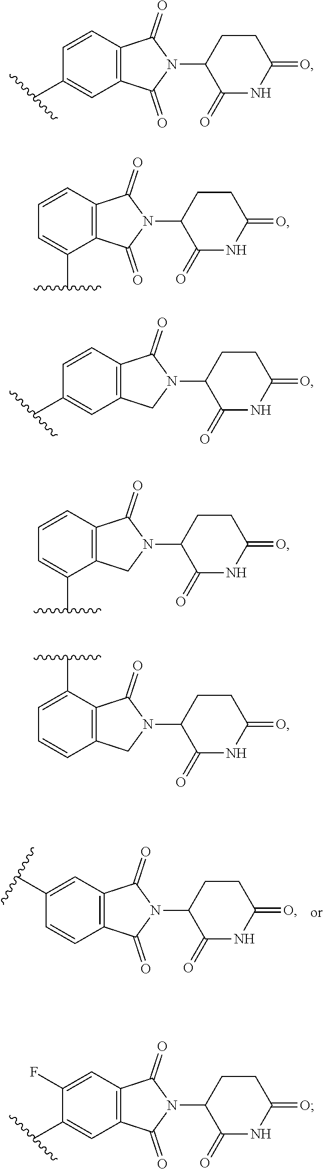

L² is an optionally substituted C₁₋₁₀ alkyl, C₁₋₁₀ heteroalkyl, C₁₋₁₀ alkynyl, 3-12 membered carbocycle, or 3-12 membered heterocycle, wherein each C₁₋₁₀ alkyl, C₁₋₁₀ alkynyl, 3-12 membered carbocycle, or 3-12 membered heterocycle of L² is optionally substituted with LBM, and/or one or more groups of R^{A10};
Wherein R^{A10} is C₁₋₁₀ alkyl, C₁₋₁₀ alkynyl, 3-12 membered carbocycle, or 3-12 membered heterocycle is independently optionally substituted with LBM, and/or one or more of the substituent groups selected from CH₃, OH, OCH₃ and F.
X¹¹ is

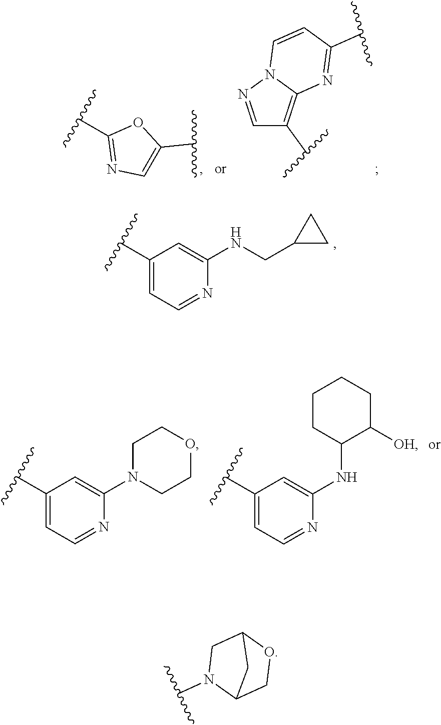

In some embodiments of any of the aspects described herein, the compound is:

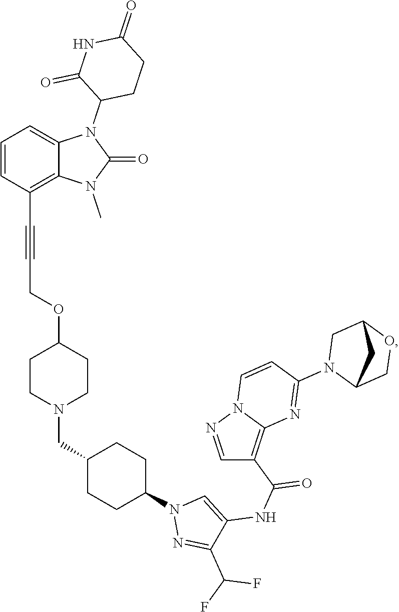 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

or a pharmaceutically acceptable salt thereof.

In some embodiments of any of the aspects described herein, the compound is N-(2-((1r,4r)-4-((6-(2-((2-(2,6-dioxopiperidin-3-yl)-1,3-dioxoisoindolin-4-yl)amino)ethyl)-2-azaspiro[3.3]heptan-2-yl)methyl)cyclohexyl)-5-(2-hydroxypropan-2-yl)benzo[d]thiazol-6-yl)-6-(trifluoromethyl)picolinamide.

In some embodiments of any of the aspects described herein, the compound is:

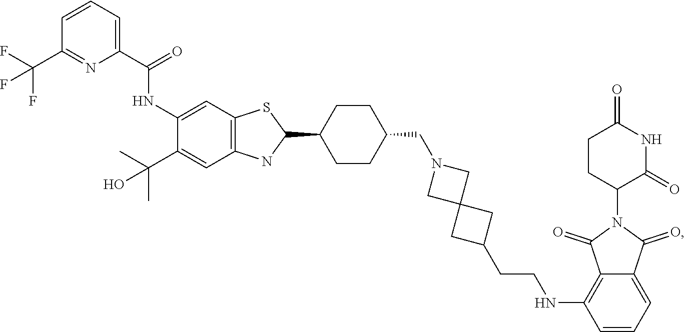 [SMILES: CC(C)(O)c1cc2c(cc1NC(=O)c1cccc(C(F)(F)F)n1)SC([C@H]1CC[C@H](CN3CC4(CC(CCNc5cccc6c5C(=O)N(C5CCC(=O)NC5=O)C6=O)C4)C3)CC1)N2]

or a pharmaceutically acceptable salt thereof.

In some embodiments of any of the aspects described herein, the compound is:

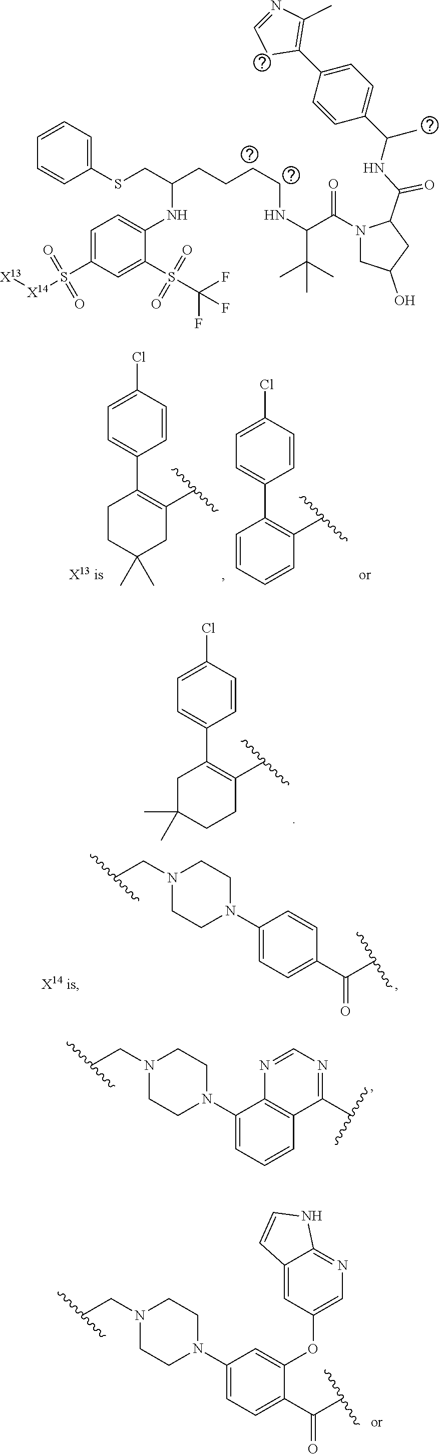

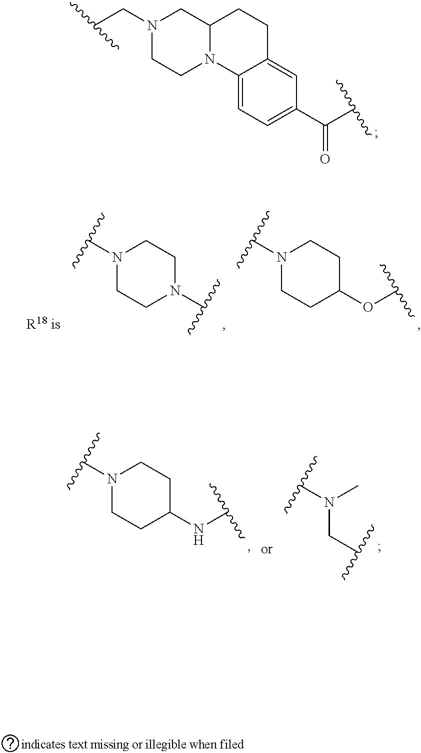

R¹⁹ is H or CH₃;
L³ is C(O)—(CH2)ₘ-O(CH2)ₙ-O(CH2)ₒ-O(CH2)ₚ-O(CH2)_{q}-O(CH2)ᵣ-OCH2-C(O), C(O)—(CH2)ₘ-O(CH2)ₙ-O(CH2)ₒ-O(CH2)ₚ-O(CH2)_{q}-OCH2-C(O), C(O)—(CH2)ₘ-O(CH2)ₙ-O(CH2)ₒ-O(CH2)ₚ-OCH2-C(O), C(O)—(CH2)ₘ-O(CH2)ₙ-O(CH2)ₒ-O(CH2)ᵣ-OCH2-C(O), C(O)—(CH2)ₘ-OCH2-C(O), wherein m, n, o, p, q and r are 1,2,3, or 4.

In some embodiments of any of the aspects described herein, the compound is:

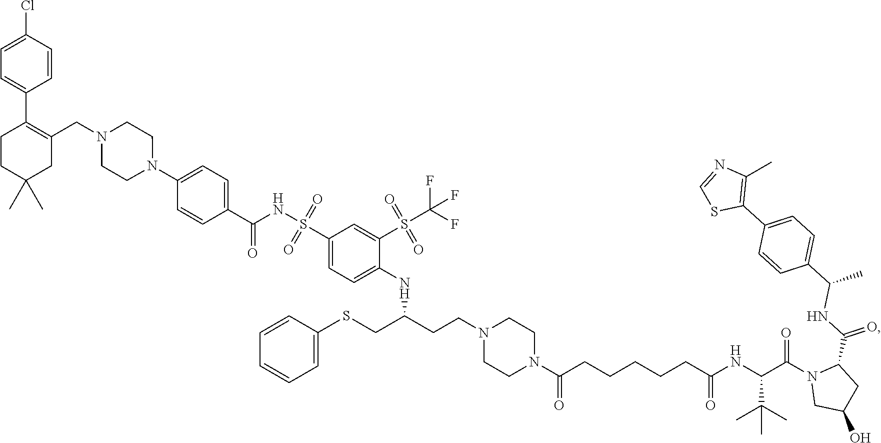 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

or a pharmaceutically acceptable salt thereof.

In some embodiments of any of the aspects described herein, the excipient includes a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof.

In some embodiments of any of the aspects described herein, the excipient is a solvent.

In some embodiments of any of the aspects described herein, the solvent is water, dichloromethane, dimethyl sulfoxide, dimethyl formamide, ethyl acetate, methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, or a combination thereof.

In some embodiments of any of the aspects described herein, the excipient is a diluent.

In some embodiments of any of the aspects described herein, the diluent is calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, saline, dry starch, cornstarch, powdered sugar, or a combination thereof.

In some embodiments of any of the aspects described herein, the excipient is a surfactant.

In some embodiments of any of the aspects described herein, the surfactant is polyvinylpyrrolidone (PVP), sodium carboxymethyl cellulose (CMC Na), PEG 4000, TWEEN®20 (polysorbate 20), TWEEN®80 (polysorbate 80), POLOXAMER®188, POLOXAMER®407, monooleate [SPAN®80], or a combination thereof.

In some embodiments of any of the aspects described herein, the excipient is a polymer.

In some embodiments of any of the aspects described herein, the polymer is poly(urethane), poly(siloxane), poly(silicone), poly(methyl methacrylate), poly(vinyl alcohol), poly(ethylene), poly(vinyl pyrrolidone) (PVP), poly(vinyl pyrrolidone) K12 (e.g., PVP K12, polyvidone, or povidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactide (PLA), polyglycolide (PGA), poly(lactide-co-glycolide) (PLGA), polyanhydrides, polyorthoester, and/or a combination thereof.

In some embodiments of any of the aspects described herein, the excipient is a cryoprotectant.

In some embodiments of any of the aspects described herein, the cryoprotectant is mannitol, PVP, PVA, PEG, amino acids such as glycine, glycerol, propylene glycol, dimethyl sulfoxide (DMSO), ethylene glycol, 2-methyl-2,4-pentadiol (MPD) or hexylene glycol, sucrose, trehalose, or a combination thereof.

In some embodiments of any of the aspects described herein, the formulation is lyophilized.

In some embodiments of any of the aspects described herein, the formulation is frozen.

In some embodiments of any of the aspects described herein, the formulation is a solution.

In some embodiments of any of the aspects described herein, the formulation is a suspension.

In some embodiments of any of the aspects described herein, the formulation is oil-based.

In some embodiments of any of the aspects described herein, the formulation is crystalline.

In some embodiments of any of the aspects described herein, the formulation is amorphous.

In some embodiments of any of the aspects described herein, the formulation is a nanosuspension.

In some embodiments of any of the aspects described herein, the nanosuspension is aqueous.

In some embodiments of any of the aspects described herein, the nanosuspension is oil based.

In some embodiments of any of the aspects described herein, the nanosuspension comprises sucrose acetate isobutyrate (SAIB).

In some embodiments of any of the aspects described herein, the formulation comprises an average particle size of between about 100 nm to about 1000 nm.

In some embodiments of any of the aspects described herein, the formulation is a microsuspension.

In some embodiments of any of the aspects described herein, the microsuspension is aqueous.

In some embodiments of any of the aspects described herein, the microsuspension is oil based.

In some embodiments of any of the aspects described herein, the oil is olive oil.

In some embodiments of any of the aspects described herein, the oil is soybean oil.

In some embodiments of any of the aspects described herein, the oil is sesame oil.

In some embodiments of any of the aspects described herein, the microsuspension has been prepared using a jet mill.

In some embodiments of any of the aspects described herein, the formulation comprises an average particle size of between about 1 μm to about 50 μm.

In some embodiments of any of the aspects described herein, the formulation has a viscosity that is between about 1 cp and about 25 cp.

In some embodiments of any of the aspects described herein, the formulation comprises TWEEN®20 (polysorbate 20).

In some embodiments of any of the aspects described herein, the formulation comprises sodium carboxymethyl cellulose (CMC Na).

In some embodiments of any of the aspects described herein, the formulation comprises poly(vinyl pyrrolidone) (PVP).

In some embodiments of any of the aspects described herein, the formulation comprises poly(vinyl pyrrolidone) K12 (PVP K12).

In some embodiments of any of the aspects described herein, the formulations comprises dimethyl sulfoxide (DMSO).

In some embodiments of any of the aspects described herein, the formulation comprises sorbitan monooleate [SPAN®80], In some embodiments of any of the aspects described herein, the formulation comprises saline.

In some embodiments of any of the aspects described herein, the formulation comprises TWEEN®20 (polysorbate 20) and saline.

In some embodiments of any of the aspects described herein, the formulation comprises poly (lactic-co-glycolic) acid (PLGA).

In some embodiments, the PLGA has an acid N-terminus.

In some embodiments of any of the aspects described herein, the formulation comprises poly (lactic-co-glycolic) acid (PLGA) microspheres.

In some embodiments of any of the aspects described herein, the PLGA microspheres are between about 1 and about 100 microns in size.

In some embodiments of any of the aspects described herein, the PLGA microspheres are between about 5 and about 100 microns in size.

In some embodiments of any of the aspects described herein, the PLGA microspheres are between about 5 and about 10 microns in size.

In some embodiments of any of the aspects described herein, the PLGA microspheres are suspended and dispersed in an excipient.

In some embodiments of any of the aspects described herein, the excipient is a solvent, diluent, surfactant, or a combination thereof.

In some embodiments of any of the aspects described herein, the microspheres are suspended and dispersed in poly(vinyl alcohol) (PVA).

In some embodiments of any of the aspects described herein, the microspheres are suspended and dispersed in POLOXAMER®188 (P-188).

In some embodiments of any of the aspects described herein, the microspheres are suspended and dispersed in POLOXAMER®407 (P-407).

In some embodiments of any of the aspects described herein, the PLGA microsphere formulation has a viscosity of between about 0 cp and about 1000 cp.

In some embodiments of any of the aspects described herein, the formulation comprises in situ PLGA gel.

In some embodiments of any of the aspects described herein, the formulation comprises a PLGA ratio of lactic acid to glycolic acid.

In some embodiments of any of the aspects described herein, the PLGA ratio is 50% lactic acid to 50% glycolic acid.

In some embodiments of any of the aspects described herein, the PLGA ratio is 65% lactic acid to 35% glycolic acid.

In some embodiments of any of the aspects described herein, the PLGA ratio is 75% lactic acid to 25% glycolic acid.

In some embodiments of any of the aspects described herein, the PLGA ratio is 85% lactic acid to 15% glycolic acid.

In some embodiments of any of the aspects described herein, the formulation comprises between about 80 mg and about 900 mg of PLGA per g of formulation.

In some embodiments of any of the aspects described herein, the PLGA has an ester N-terminus.

In some embodiments of any of the aspects described herein, the PLGA has an acid N-terminus. In some embodiments of any of the aspects described herein, the formulation comprises between about 1 mg and about 250 mg of PLGA per g of formulation.

In some embodiments of any of the aspects described herein, the formulation comprises dimethylacetamide (DMA).

In some embodiments of any of the aspects described herein, the formulation comprises dimethyl sulfoxide (DMSO).

In some embodiments of any of the aspects described herein, the formulation comprises dimethylformamide (DMF).

In some embodiments of any of the aspects described herein, the formulation comprises N-methyl-2-pryrrolidone (NMP).

In some embodiments of any of the aspects described herein, the formulation comprises an in situ sucrose acetate isobutyrate (SAIB) gel.

In one aspect, the present disclosure provides a method of treating cancer in a subject in need thereof, the method comprising administering to the subject an effective amount of a compound that reduces the level and/or activity of a protein, or a pharmaceutically acceptable salt thereof, and a pharmaceutically acceptable excipient.

In one aspect, the present disclosure provides a method of treating cancer in a subject in need thereof, the method comprising administering to the subject an effective amount of administering to the subject an effective amount of a compound that reduces the level and/or activity of a protein, or a pharmaceutically acceptable salt thereof.

In one aspect, the present disclosure provides a method of treating cancer in a subject in need thereof, the method comprising administering to the subject an effective amount of a compound that reduces the level and/or activity of BRD9, or a pharmaceutically acceptable salt thereof, and a pharmaceutically acceptable excipient. In some embodiments of any of the aspects described herein, the cancer is breast cancer, prostate cancer, colorectal cancer, pancreatic cancer, B cell Lymphoma, leukemia, T cell leukemia, NSCLC, multiple myeloma (MM), non-Hodgkin's lymphomas (NHL), melanoma, endometrial cancer, intestinal cancer, and skin cancer. In some embodiments of any of the aspects described herein, the cancer is synovial sarcoma.

In one aspect, the present disclosure provides a method of treating cancer in a subject in need thereof, the method comprising administering to the subject an effective amount of a compound that reduces the level and/or activity of CBP, or a pharmaceutically acceptable salt thereof, and a pharmaceutically acceptable excipient. In some embodiments, the cancer is osteosarcoma, colorectal cancer, bladder cancer, gastric cancer, breast cancer, head and neck cancer, prostate cancer, acute leukemias, ovarian cancer, neuroblastoma, myelofibrosis, lymphoma, leukemia, esophogeal, stomach, or lung cancer. In some embodiments, the cancer is gastric cancer. In some embodiments, the cancer is colorectal cancer. In some embodiments, the cancer is bladder cancer.

In one aspect, the present disclosure provides a method of treating cancer in a subject in need thereof, the method comprising administering to the subject an effective amount of a compound that reduces the level and/or activity of EP300, or a pharmaceutically acceptable salt thereof, and a pharmaceutically acceptable excipient. In some embodiments of any of the aspects described herein, the cancer is osteosarcoma, colorectal cancer, bladder cancer, gastric cancer, breast cancer, head and neck cancer, prostate cancer, acute leukemias, ovarian cancer, neuroblastoma, myeloma, skin, endometrial, esophageal, cervical, gastric, lymphoma, leukemia, esophogeal, stomach, lung cancer, or non-small cell lung cancer. In some embodiments, the cancer is prostate cancer.

In one aspect, the present disclosure provides a method of treating cancer in a subject in need thereof, the method comprising administering to the subject an effective amount of a compound that reduces the level and/or activity of estrogen receptor, or a pharmaceutically acceptable salt thereof, and a pharmaceutically acceptable excipient. In some embodiments of any of the aspects described herein, the cancer is breast cancer.

In one aspect, the present disclosure provides a method of treating cancer in a subject in need thereof, the method comprising administering to the subject an effective amount of a compound that reduces the level and/or activity of androgen receptor, or a pharmaceutically acceptable salt thereof, and a pharmaceutically acceptable excipient. In some embodiments of any of the aspects described herein, the cancer is prostate cancer.

In one aspect, the present disclosure provides a method of treating cancer in a subject in need thereof, the method comprising administering to the subject an effective amount of a compound that reduces the level and/or activity of IRAK4, or a pharmaceutically acceptable salt thereof, and a pharmaceutically acceptable excipient. In some embodiments of any of the aspects described herein, the cancer is pancreatic cancer. In some embodiments of any of the aspects described herein, the cancer is colorectal cancer.

In one aspect, the present disclosure provides a method of treating cancer in a subject in need thereof, the method comprising administering to the subject an effective amount of a compound that reduces the level and/or activity of IRAK4 and/or IMiD, or a pharmaceutically acceptable salt thereof, and a pharmaceutically acceptable excipient. In some embodiments of any of the aspects described herein, the cancer is relapsed/refractory B cell lymphomas. In some embodiments of any of the aspects described herein, the cancer is MYD88-mutant diffuse large B cell lymphoma (DLBCL).

In one aspect, the present disclosure provides a method of treating cancer in a subject in need thereof, the method comprising administering to the subject an effective amount of a compound that reduces the level and/or activity of STAT3, or a pharmaceutically acceptable salt thereof, and a pharmaceutically acceptable excipient. In some embodiments of any of the aspects described herein, the cancer is T cell lymphomas. In some embodiments of any of the aspects described herein, the cancer is leukemia.

In one aspect, the present disclosure provides a method of treating cancer in a subject in need thereof, the method comprising administering to the subject an effective amount of a compound that reduces the level and/or activity of MDM2, or a pharmaceutically acceptable salt thereof, and a pharmaceutically acceptable excipient. In some embodiments of any of the aspects described herein, the cancer is leukemia.

In one aspect, the present disclosure provides a method of treating cancer in a subject in need thereof, the method comprising administering to the subject an effective amount of a compound that reduces the level and/or activity of BCLXL, or a pharmaceutically acceptable salt thereof, and a pharmaceutically acceptable excipient. In some embodiments of any of the aspects described herein, the cancer is T cell lymphoma.

In one aspect, the present disclosure provides a method of treating cancer in a subject in need thereof, the method comprising administering to the subject an effective amount of a compound that reduces the level and/or activity of BTK, or a pharmaceutically acceptable salt thereof, and a pharmaceutically acceptable excipient. In some embodiments of any of the aspects described herein, the cancer is leukemia. In some embodiments of any of the aspects described herein, the cancer is lymphoma.

In one aspect, the present disclosure provides a method of treating cancer in a subject in need thereof, the method comprising administering to the subject an effective amount of a compound that reduces the level and/or activity of BTK and/or IMiD, or a pharmaceutically acceptable salt thereof, and a pharmaceutically acceptable excipient. In some embodiments of any of the aspects described herein, the cancer is leukemia. In some embodiments of any of the aspects described herein, the cancer is lymphoma.

In one aspect, the present disclosure provides a method of treating cancer in a subject in need thereof, the method comprising administering to the subject an effective amount of a compound that reduces the level and/or activity of TRK, or a pharmaceutically acceptable salt thereof, and a pharmaceutically acceptable excipient. In some embodiments of any of the aspects described herein, the cancer is non-small cell lung cancer. In some embodiments of any of the aspects described herein, the cancer is breast cancer. In some embodiments of any of the aspects described herein, the cancer is pancreatic cancer.

In one aspect, the present disclosure provides a method of treating cancer in a subject in need thereof, the method comprising administering to the subject an effective amount of a compound that reduces the level and/or activity of BRM, or a pharmaceutically acceptable salt thereof, and a pharmaceutically acceptable excipient. In some embodiments of any of the aspects described herein, the cancer is non-small cell lung cancer.

In one aspect, the present disclosure provides a method of treating cancer in a subject in need thereof, the method comprising administering to the subject an effective amount of a compound that reduces the level and/or activity of IKZF1 and/or IKZF3, or a pharmaceutically acceptable salt thereof, and a pharmaceutically acceptable excipient. In some embodiments of any of the aspects described herein, the cancer is non-small cell lung cancer.

In one aspect, the present disclosure provides a method of treating cancer in a subject in need thereof, the method comprising administering to the subject an effective amount of a compound that reduces the level and/or activity of BRAF V600X, or a pharmaceutically acceptable salt thereof, and a pharmaceutically acceptable excipient. In some embodiments of any of the aspects described herein, the cancer is non-small cell lung cancer.

In one aspect, the present disclosure provides a method of treating cancer in a subject in need thereof, the method comprising administering to the subject an effective amount of a compound that reduces the level and/or activity of EGFR L585R, or a pharmaceutically acceptable salt thereof, and a pharmaceutically acceptable excipient. In some embodiments of any of the aspects described herein, the cancer is non-small cell lung cancer.

In some embodiments, the compound is any one of compounds 1-9 in Table 1.

TABLE 1
Compounds 1-9

| | |
|---|---|
| 1 | 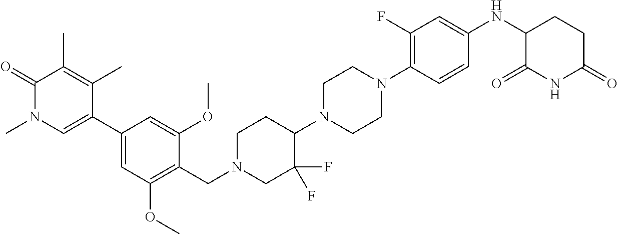 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1] |
| | CFT8634-iso |
| 2 | 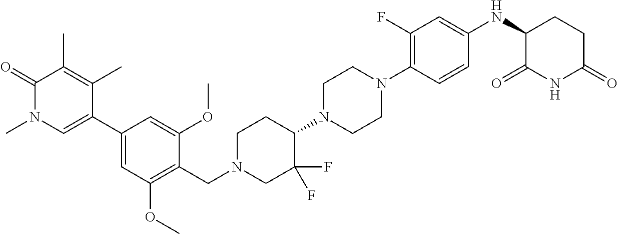 [SMILES: COc1cc(-c2cn(C)c(=O)c(C)c2C)cc(OC)c1CN1CC[C@H](N2CCN(c3ccc(N[C@H]4CCC(=O)NC4=O)cc3F)CC2)C(F)(F)C1] |
| | CFT8634 |
| 3 | 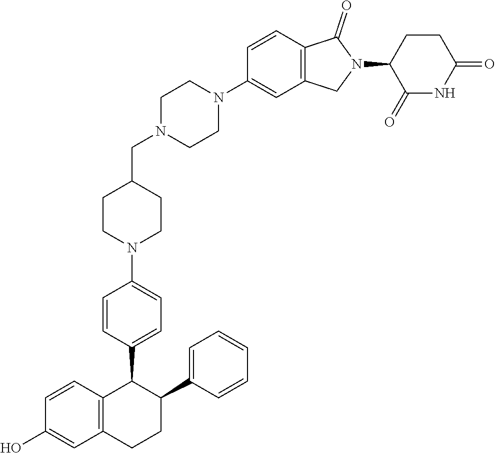 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| | ARV471 |
| 4 | 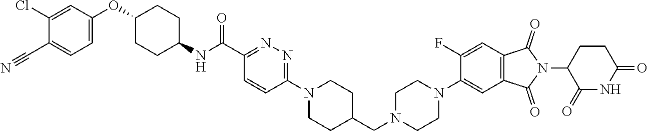 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl] |
| | ARV110 |
| 5 | 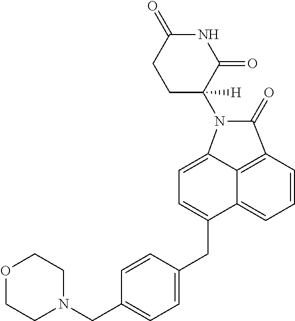 [SMILES: O=C1CC[C@H](N2C(=O)c3cccc4c(Cc5ccc(CN6CCOCC6)cc5)ccc2c34)C(=O)N1] |
| | CFT7455 |
| 6 | 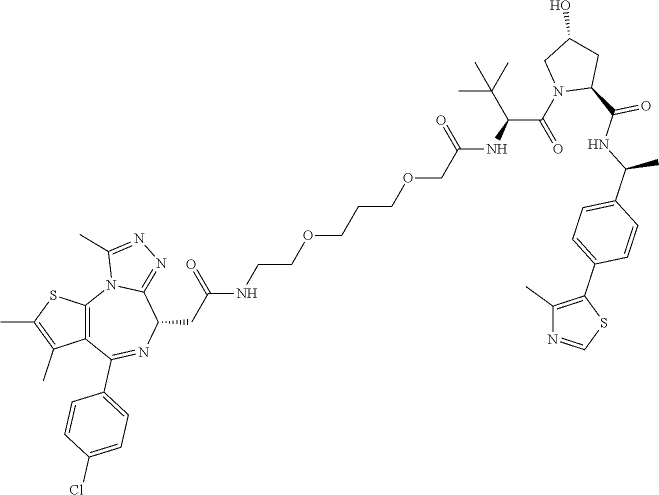 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1] |
| | ARV 776 |
| 7 | 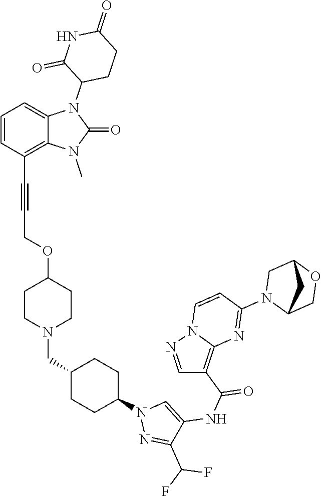 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21] |
| | KT-474 |
| 8 | 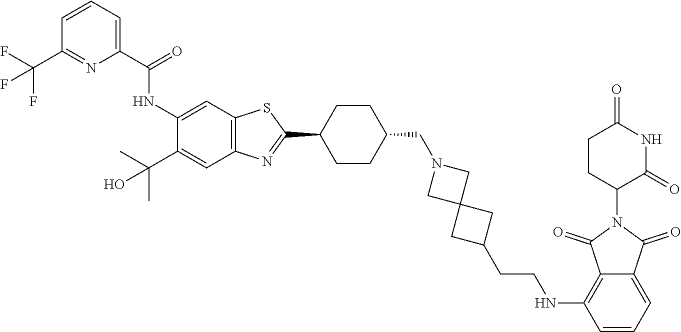 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)sc2cc1NC(=O)c1cccc(C(F)(F)F)n1] |
| | KT 413 |
| 9 | 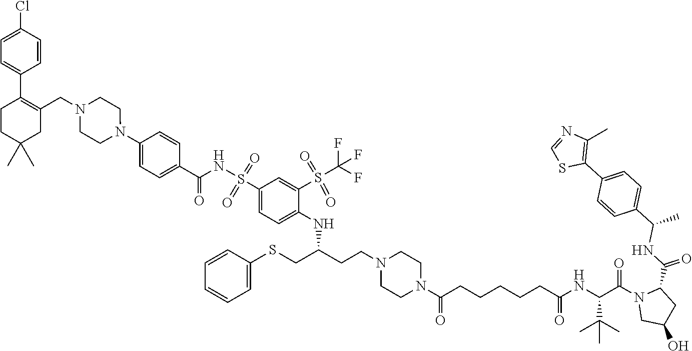 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| | DT-2216 |

In one aspect, the present disclosure provides a method of treating synovial sarcoma in a subject in need thereof, the method comprising administering to the subject an effective amount of administering to the subject an effective amount of a compound that reduces the level and/or activity of BRD9, or a pharmaceutically acceptable salt thereof.

In one aspect, the present disclosure provides a method of treating cancer in a subject in need thereof, the method comprising administering to the subject an effective amount of the compound

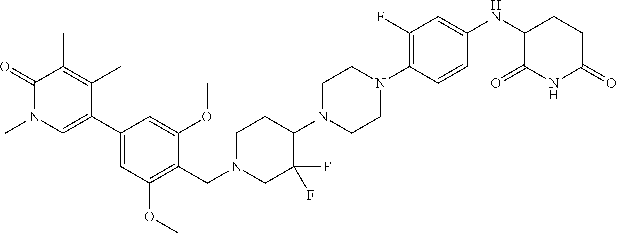 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1]

or a pharmaceutically acceptable salt thereof, and a pharmaceutically acceptable excipient. In some embodiments of any of the aspects described herein, the cancer is synovial sarcoma.

In one aspect, the present disclosure provides a method of treating cancer in a subject in need thereof, the method comprising administering to the subject an effective amount of the compound:

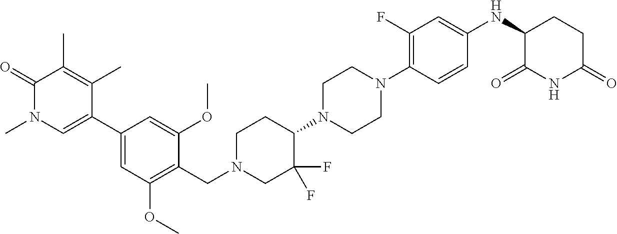 [SMILES: COc1cc(-c2cn(C)c(=O)c(C)c2C)cc(OC)c1CN1CC[C@H](N2CCN(c3ccc(N[C@H]4CCC(=O)NC4=O)cc3F)CC2)C(F)(F)C1]

or a pharmaceutically acceptable salt thereof, and a pharmaceutically acceptable excipient. In some embodiments of any of the aspects described herein, the cancer is synovial sarcoma.

In one aspect, the present disclosure provides a method of treating cancer in a subject in need thereof, the method comprising administering to the subject an effective amount of the compound:

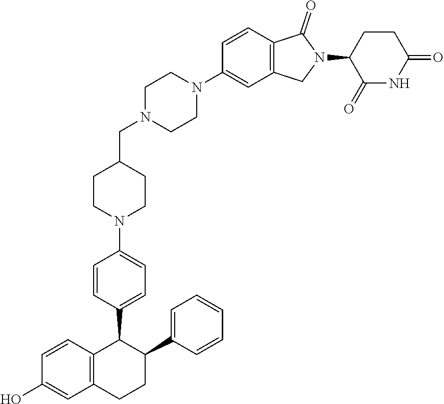 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, and a pharmaceutically acceptable excipient. In some embodiments of any of the aspects described herein, the cancer is breast cancer.

In one aspect, the present disclosure provides a method of treating cancer in a subject in need thereof, the method comprising administering to the subject an effective amount of the compound:

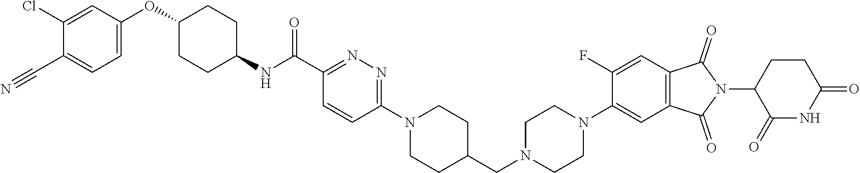 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt thereof, and a pharmaceutically acceptable excipient. In some embodiments of any of the aspects described herein, the cancer is prostate cancer.

In one aspect, the present disclosure provides a method of treating cancer in a subject in need thereof, the method comprising administering to the subject an effective amount of the compound:

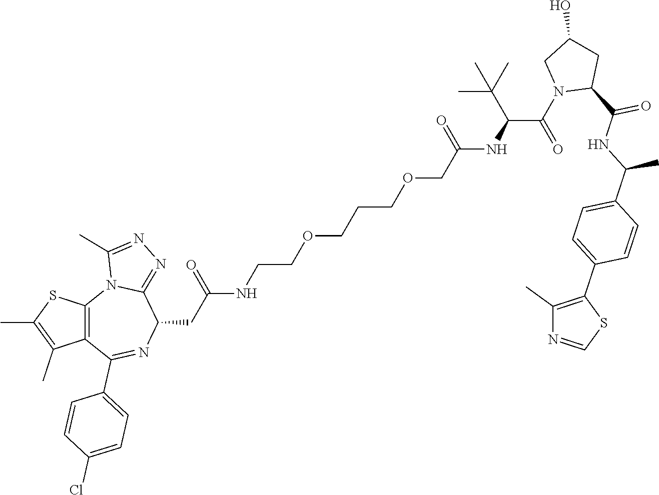 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1]

or a pharmaceutically acceptable salt thereof, and a pharmaceutically acceptable excipient. In some embodiments of any of the aspects described herein, the cancer is prostate cancer.

In one aspect, the present disclosure provides a method of treating cancer in a subject in need thereof, the method comprising administering to the subject an effective amount of the compound:

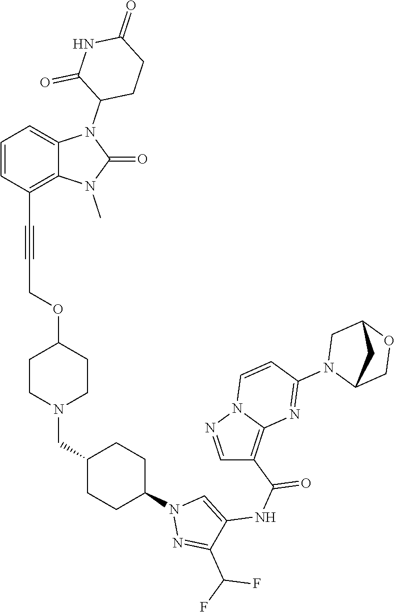 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

or a pharmaceutically acceptable salt thereof, and a pharmaceutically acceptable excipient. In some embodiments of any of the aspects described herein, the cancer is pancreatic cancer. In some embodiments of any of the aspects described herein, the cancer is colorectal cancer.

In one aspect, the present disclosure provides a method of treating cancer in a subject in need thereof, the method comprising administering to the subject an effective amount of the compound:

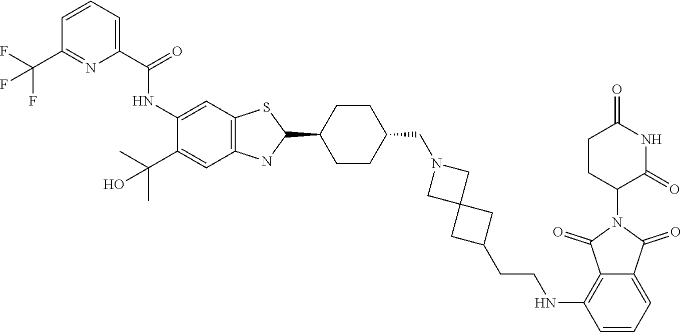 [SMILES: CC(C)(O)c1cc2c(cc1NC(=O)c1cccc(C(F)(F)F)n1)SC([C@H]1CC[C@H](CN3CC4(CC(CCNc5cccc6c5C(=O)N(C5CCC(=O)NC5=O)C6=O)C4)C3)CC1)N2]

or a pharmaceutically acceptable salt thereof, and a pharmaceutically acceptable excipient. In some embodiments of any of the aspects described herein, the cancer is B Cell lymphoma.

In one aspect, the present disclosure provides a method of treating cancer in a subject in need thereof, the method comprising administering to the subject an effective amount of the compound:

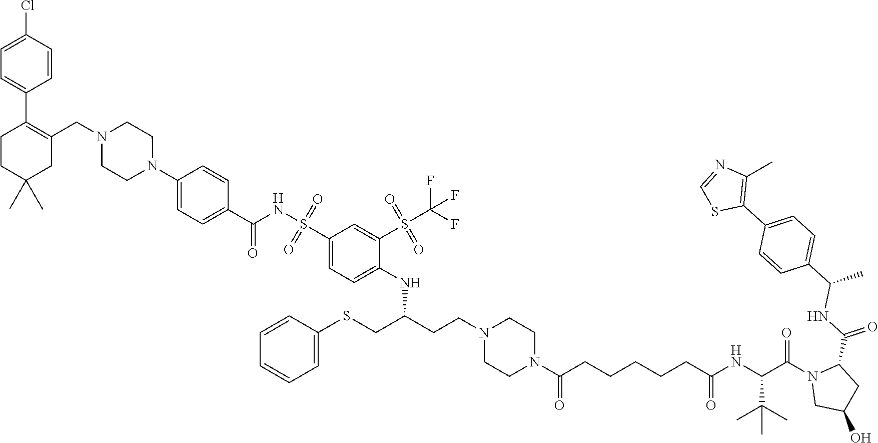 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

or a pharmaceutically acceptable salt thereof, and a pharmaceutically acceptable excipient. In some embodiments of any of the aspects described herein, the cancer is T cell lymphoma.

In one aspect, the present disclosure provides a method of treating cancer in a subject in need thereof, the method comprising administering to the subject an effective amount of the compound:

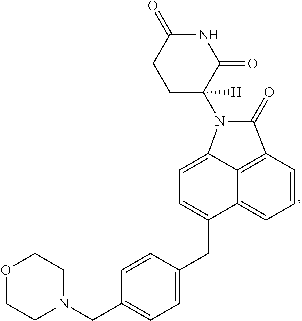 [SMILES: O=C1CC[C@H](N2C(=O)c3cccc4c(Cc5ccc(CN6CCOCC6)cc5)ccc2c34)C(=O)N1]

or a pharmaceutically acceptable salt thereof, and a pharmaceutically acceptable excipient. In some embodiments of any of the aspects described herein, the cancer is T cell lymphoma.

TABLE 2
Compounds and their Target Protein and Indication

| Compound | Target Protein | Indications |
|---|---|---|
| CFT8634-Iso (1) | BRD9 | Synovial Sarcoma |
| CFT8634 (2) | BRD9 | Synovial Sarcoma |
| ARV471 (3) | Estrogen Receptor | Breast Cancer |
| ARV110 (4) | Androgen Receptor | Prostate Cancer |
| CFT-7455 (5) | IKZF1 and/or | multiple myeloma (MM) |
| | IKZF3 | and non-Hodgkin's |
| | | lymphomas (NHL) |
| ARV766 (6) | Androgen Receptor | Prostate Cancer |
| KT-474 (7) | IRAK4 | Pancreatic and |
| | | Colorectal Cancer |
| KT-413 (8) | IRAK4 and/or IMiD | B Cell Lymphoma |
| DT-2216 (9) | BCLXL | T Cell Leukemia |

In some embodiments of any of the aspects described herein, the administering comprises combining the compound with an excipient to form a solution and administering the resulting solution.

In some embodiments of any of the aspects described herein, the administering comprises coupling a vial comprising the compound and a syringe comprising an excipient, transferring the contents of the syringe into the vial, mixing to form a solution, and administering the resulting solution.

In some embodiments of any of the aspects described herein, the administering comprises coupling a syringe comprising the compound and a syringe comprising an excipient, mixing to form a solution, and administering the resulting solution.

In some embodiments of any of the aspects described herein, the administering comprises coupling a vial comprising the compound and a syringe comprising PLGA and an excipient, transferring the contents of the syringe into the vial, mixing to form a solution, and administering the resulting solution.

In some embodiments of any of the aspects described herein, the administering comprises coupling a syringe comprising the compound and a syringe comprising PLGA and an excipient, mixing to form a solution, and administering the resulting solution.

In some embodiments of any of the aspects described herein, the administering comprises mixing the formulation to form a suspension and administering the resulting suspension.

In some embodiments of any of the aspects described herein, the administering comprises mixing the formulation to form a nanosuspension and administering the resulting nanosuspension.

In some embodiments of any of the aspects described herein, the administering comprises mixing the formulation to form a microsuspension and administering the resulting microsuspension.

In some embodiments of any of the aspects described herein, the effective amount of the compound is administered at a dose of between about 0.1 mg/kg and about 1000 mg/kg.

In some embodiments of any of the aspects described herein, the effective amount of the compound is administered at a dose of between about 0.1 mg/mL and about 1000 mg/mL.

In some embodiments of any of the aspects described herein, the effective amount of the compound is administered at a dose of between about 0.1 mg and about 1000 mg.

In some embodiments of any of the aspects described herein, the formulation is administered in a dose of between about 0.25 mL and about 3 mL.

In some embodiments of any of the aspects described herein, the effective amount of the compound is administered in a dose of between about 0.1 mg/m2 and about 1000 mg/m2.

In some embodiments of any of the aspects described herein, the formulation is administered at least once every week.

In some embodiments of any of the aspects described herein, the formulation is administered at least once every two weeks.

In some embodiments of any of the aspects described herein, the formulation is administered at least once every three weeks.

In some embodiments of any of the aspects described herein, the formulation is administered at least once every four weeks.

In some embodiments of any of the aspects described herein, the formulation is administered at least once every month.

In some embodiments of any of the aspects described herein, the formulation is administered at least once every six weeks.

In some embodiments of any of the aspects described herein, the formulation is administered at least once every eight weeks.

In some embodiments of any of the aspects described herein, the formulation is administered at least once every 2 months.

In some embodiments of any of the aspects described herein, the formulation is administered to the subject in a one-week dosing cycle.

In some embodiments of any of the aspects described herein, the formulation is administered to the subject in a two-week dosing cycle.

In some embodiments of any of the aspects described herein, the formulation is administered to the subject in a three-week dosing cycle.

In some embodiments of any of the aspects described herein, the formulation is administered to the subject in a four-week dosing cycle.

In some embodiments of any of the aspects described herein, the formulation is administered to the subject in a one-month dosing cycle.

In some embodiments of any of the aspects described herein, the formulation is administered to the subject in a six-week dosing cycle.

In some embodiments of any of the aspects described herein, the formulation is administered to the subject in an eight-week dosing cycle.

In some embodiments of any of the aspects described herein, the formulation is administered to the subject in a two-month dosing cycle.

In some embodiments of any of the aspects described herein, the formulation is administered subcutaneously.

In some embodiments of any of the aspects described herein, the formulation is administered intramuscularly.

In one aspect, the present disclosure provides a kit comprising a pre-filled syringe comprising a pharmaceutically acceptable composition comprising a long-acting formulation of a compound that reduces the level and/or activity of a protein, or a pharmaceutically acceptable salt thereof, and a pharmaceutically acceptable excipient; and a package insert with instructions to perform administration.

### Chemical Terms

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(RN1)2, wherein each RN1 is, independently, H, OH, NO2, N(RN2)2, SO2ORN2, SO2RN2, SORN2, an N-protecting group, alkyl, alkoxy, aryl, arylalkyl, cycloalkyl, acyl (e.g., acetyl, trifluoroacetyl, or others described herein), wherein each of these recited RN1 groups can be optionally substituted; or two RN1 combine to form an alkylene or heteroalkylene, and wherein each RN2 is, independently, H, alkyl, or aryl. The amino groups of the compounds described herein can be an unsubstituted amino (i.e., —NH2) or a substituted amino (i.e., —N(RN1)2).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Unsubstituted arylalkyl groups contain from 7 to 30 carbons (e.g., from 7 to 16 or from 7 to 20 carbons, such as C1-C6 alkyl C6-C10 aryl, C1-C10 alkyl C6-C10 aryl, or C1-C20 alkyl C6-C10 aryl), such as, benzyl and phenethyl. In some embodiments, the alkyl and the aryl each are further substituted with 1, 2, 3, or 4 substituent groups, valency permitting, as defined herein for the respective groups.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals. A carbocyclylene is a divalent carbocyclyl group.

The term “cycloalkyl,” as used herein, refers to a saturated, non-aromatic, and monovalent mono-di-, or tricyclic radical of 3 to 10, preferably 3 to 6 carbon atoms. The cycloalkyl group may be fully saturated or contain 1 or more double or triple bonds, provided that no ring is aromatic. This term is further exemplified by radicals such as cyclopropyl, cyclobutyl, cyclopentyl, cyclohexyl, cycloheptyl, norbornyl, and adamantyl. The term “cycloalkoxy” as used herein, refers to cycloalkyl-O— groups (e.g., cyclopropoxy and cyclobutoxy).

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Unsubstituted heteroarylalkyl groups contain from 7 to 30 carbons (e.g., from 7 to 16 or from 7 to 20 carbons, such as C1-C6 alkyl C2-C9 heteroaryl, C1-C10 alkyl C2-C9 heteroaryl, or C1-C20 alkyl C2-C9 heteroaryl). In some embodiments, the alkyl and the heteroaryl each are further substituted with 1, 2, 3, or 4 substituent groups, valency permitting, as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

A heterocyclylene is a divalent heterocyclyl group.

The term “hydroxyl,” as used herein, represents an —OH group.

The term “thiol,” as used herein, represents an —SH group.

The term “carbonyl,” as used herein, represents an —C(O)— group.

The term “thiocarbonyl,” as used herein, represents an —C(S)— group.

The term “sulfonyl,” as used herein, represents an —S(O)₂— group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide 35 of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “adult soft tissue sarcoma” refers to a sarcoma that develops in the soft tissues of the body, typically in adolescent and adult subjects (e.g., subjects who are at least 10 years old, 11 years old, 12 years old, 13 years old, 14 years old, 15 years old, 16 years old, 17 years old, 18 years old, or 19 years old). Non-limiting examples of adult soft tissue sarcoma include, but are not limited to, synovial sarcoma, fibrosarcoma, malignant fibrous histiocytoma, dermatofibrosarcoma, liposarcoma, leiomyosarcoma, hemangiosarcoma, Kaposi's sarcoma, lymphangiosarcoma, malignant peripheral nerve sheath tumor/neurofibrosarcoma, extraskeletal chondrosarcoma, extraskeletal osteosarcoma, extraskeletal myxoid chondrosarcoma, and extraskeletal mesenchymal chondrosarcoma.

As used herein, the term “BAF complex” refers to the BRG1- or HRBM-associated factors complex in a human cell.

As used herein, the term “BAF complex-related disorder” refers to a disorder that is caused or affected by the level and/or activity of a BAF complex.

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18.

As used herein, the term “BRD9” refers to bromodomain-containing protein 9, a component of the BAF (BRG1- or BRM-associated factors) complex, a SWI/SNF ATPase chromatin remodeling complex, and belongs to family IV of the bromodomain-containing proteins. BRD9 is encoded by the BRD9 gene, the nucleic acid sequence of which is set forth in SEQ ID NO: 1. The term “BRD9” also refers to natural variants of the wild-type BRD9 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BRD9, which is set forth in SEQ ID NO: 2.

SEQ ID NO: 1
atgggcaagaagcacaagaagcacaaggccgagtggcgctcgtcctacga
ggattatgccgacaagcccctggagaagcctctaaagctagtcctgaagg
tcggaggaagtgaagtgactgaactctcaggatccggccacgactccagt
tactatgatgacaggtcagaccatgagcgagagaggcacaaagaaaagaa
aaagaagaagaagaagaagtccgagaaggagaagcatctggacgatgagg
aaagaaggaagcgaaaggaagagaagaagcggaagcgagagagggagcac
tgtgacacggagggagaggctgacgactttgatcctgggaagaaggtgga
ggtggagccgcccccagatcggccagtccgagcgtgccggacacagccag
ccgaaaatgagagcacacctattcagcaactcctggaacacttcctccgc
cagcttcagagaaaagatccccatggattttttgcttttcctgtcacgga
tgcaattgctcctggatattcaatgataataaaacatcccatggattttg
gcaccatgaaagacaaaattgtagctaatgaatacaagtcagttacggaa
tttaaggcagatttcaagctgatgtgtgataatgcaatgacatacaatag
gccagataccgtgtactacaagttggcgaagaagatccttcacgcaggct
ttaagatgatgagcaaacaggcagctcttttgggcaatgaagatacagct
gttgaggaacctgtccctgaagttgtaccagtacaagtagaaactgccaa
gaaatccaaaaagccgagtagagaagttatcagctgcatgtttgagcctg
aagggaatgcctgcagcttgacggacagtaccgcagaggagcacgtgctg
gcgctggtggagcacgcagctgacgaagctcgggacaggatcaaccggtt
cctcccaggcggcaagatgggctatctgaagaggaacggggacgggagcc
tgctctacagcgtggtcaacacggccgagccggacgctgatgaggaggag
acccacccggtggacttgagctcgctctccagtaagctactcccaggctt
caccacgctgggcttcaaagacgagagaagaaacaaagtcacctttctct
ccagtgccactactgcgctttcgatgcagaataattcagtatttggcgac
ttgaagtcggacgagatggagctgctctactcagcctacggagatgagac
aggcgtgcagtgtgcgctgagcctgcaggagtttgtgaaggatgctggga
gctacagcaagaaagtggtggacgacctcctggaccagatcacaggcgga
gaccactctaggacgctcttccagctgaagcagagaagaaatgttcccat
gaagcctccagatgaagccaaggttggggacaccctaggagacagcagca
gctctgttctggagttcatgtcgatgaagtcctatcccgacgtttctgtg
gatatctccatgctcagctctctggggaaggtgaagaaggagctggaccc
tgacgacagccatttgaacttggatgagacgacgaagctcctgcaggacc
tgcacgaagcacaggcggagcgcggcggctctcggccgtcgtccaacctc
agctccctgtccaacgcctccgagagggaccagcaccacctgggaagccc
ttctcgcctgagtgtcggggagcagccagacgtcacccacgacccctatg
agtttcttcagtctccagagcctgcggcctctgccaagacctaa
SEQ ID NO: 2:
MGKKHKKHKAEWRSSYEDYADKPLEKPLKLVLKVGGSEVTELSGSGHDSS
YYDDRSDHERERHKEKKKKKKKKSEKEKHLDDEERRKRKEEKKRKREREH
CDTEGEADDFDPGKKVEVEPPPDRPVRACRTQPAENESTPIQQLLEHFLR
QLQRKDPHGFFAFPVTDAIAPGYSMIIKHPMDFGTMKDKIVANEYKSVTE
FKADFKLMCDNAMTYNRPDTVYYKLAKKILHAGFKMMSKQAALLGNEDTA
VEEPVPEVVPVQVETAKKSKKPSREVISCMFEPEGNACSLTDSTAEEHVL
ALVEHAADEARDRINRFLPGGKMGYLKRNGDGSLLYSVVNTAEPDADEEE
THPVDLSSLSSKLLPGFTTLGFKDERRNKVTFLSSATTALSMQNNSVFGD
LKSDEMELLYSAYGDETGVQCALSLQEFVKDAGSYSKKVVDDLLDQITGG
DHSRTLFQLKQRRNVPMKPPDEAKVGDTLGDSSSSVLEFMSMKSYPDVSV
DISMLSSLGKVKKELDPDDSHLNLDETTKLLODLHEAQAERGGSRPSSNL
SSLSNASERDOHHLGSPSRLSVGEQPDVTHDPYEFLQSPEPAASAKT

As used herein, the term “BRD9-related disorder” refers to a disorder that is caused or affected by the level and/or activity of BRD9. The term “cancer” refers to a condition caused by the proliferation of malignant neoplastic cells, such as tumors, neoplasms, carcinomas, sarcomas, leukemias, and lymphomas.

As used herein, the term “CBP” refers to the Creb-binding protein in a human cell.

As used herein, the term “CBP-related disorder” refers to a disorder that is caused or affected by the level of activity of CBP.

As used herein, the term “CBP loss of function mutation” refers to a mutation in CBP that leads to the protein having diminished activity (e.g., at least 1% reduction in CBP activity, for example 2%, 5%, 10%, 25%, 50%, or 100% reduction in CBP activity). Exemplary CBP loss of function mutations include, but are not limited to, a homozygous CBP mutation and chromosomal translocations.

As used herein, the term “CBP loss of function disorder” refers to a disorder (e.g., cancer) that exhibits a reduction in CBP activity (e.g., at least 1% reduction in CBP activity, for example 2%, 5%, 10%, 25%, 50%, or 100% reduction in CBP activity).

By “reducing the activity of CBP” is meant reducing the level of an activity related to CBP, or a related downstream effect. The activity level of a CBP may be measured using any method known in the art, e.g., HiBit assay. By “reducing the level of CBP,” is meant decreasing the level of CBP in a cell or subject. The level of CBP may be measured using any method known in the art.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

A “compound of the present disclosure” and similar terms as used herein, whether explicitly noted or not, refers to compounds useful for treating BAF-related disorders (e.g., cancer) described herein, including, e.g., 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione and the compounds disclosed in International Patent Publication Nos. WO/2021/155225, WO/2021/022163, WO/2020/160198, WO/2020/160196, WO/2020/160193, WO/2020/160192, and WO/2019/152440 (the contents of which are incorporated herein by reference in their entirety), as well as salts (e.g., pharmaceutically acceptable salts), solvates, hydrates, stereoisomers (including atropisomers), and tautomers thereof. Those skilled in the art will appreciate that certain compounds described herein can exist in one or more different isomeric (e.g., stereoisomers, geometric isomers, atropisomers, and tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination. Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

As used herein, the term “degrader” refers to a small molecule compound including a degradation moiety, wherein the compound interacts with a protein (e.g., BRD9, CBP, or EP300) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRD9, CBP, or EP300. In one example, the moiety binds to a protease or a ubiquitin ligase that metabolizes the protein, e.g., BRD9, CBP, or EP300.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

As used herein, the terms “effective amount,” “therapeutically effective amount,” and “a “sufficient amount” of an agent that reduces the level and/or activity of BRD9 (e.g., in a cell or a subject) described herein, unless specified otherwise, refer to a quantity sufficient to, when administered to the subject, including a human, decrease a BRD9 immunohistochemistry score of the subject or an amount sufficient to reduce the level of BRD9 expression in the subject, as described herein. An “effective amount” may also refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRD9 sufficient to achieve a treatment response as compared to the response obtained without administration of the agent that reduces the level and/or activity of BRD9. The amount of a given agent that reduces the level and/or activity of BRD9 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRD9 of the present disclosure is an amount which results in a beneficial or desired result in a subject, e.g., as compared to a control. As defined herein, a therapeutically effective amount of an agent that reduces the level and/or activity of BRD9 of the present disclosure may be readily determined by one of ordinary skill by routine methods known in the art. Dosage regimen may be adjusted to provide the optimum therapeutic response.

As used herein, the terms “effective amount,” “therapeutically effective amount,” and “a “sufficient amount” of an agent that reduces the level and/or activity of CBP (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of CBP sufficient to achieve a treatment response as compared to the response obtained without administration of the agent that reduces the level and/or activity of CBP. The amount of a given agent that reduces the level and/or activity of CBP described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of CBP of the present disclosure is an amount which results in a beneficial or desired result in a subject as compared to a control. As defined herein, a therapeutically effective amount of an agent that reduces the level and/or activity of CBP of the present disclosure may be readily determined by one of ordinary skill by routine methods known in the art. Dosage regimen may be adjusted to provide the optimum therapeutic response.

As used herein, the terms “effective amount,” “therapeutically effective amount,” and “a “sufficient amount” of an agent that reduces the level and/or activity of EP300 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of EP300 sufficient to achieve a treatment response as compared to the response obtained without administration of the agent that reduces the level and/or activity of EP300. The amount of a given agent that reduces the level and/or activity of EP300 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of EP300 of the present disclosure is an amount which results in a beneficial or desired result in a subject as compared to a control. As defined herein, a therapeutically effective amount of an agent that reduces the level and/or activity of EP300 of the present disclosure may be readily determined by one of ordinary skill by routine methods known in the art. Dosage regimen may be adjusted to provide the optimum therapeutic response.

As used herein, the term “EP300” refers to the EP300 protein in a human cell.

As used herein, the term “EP300-related disorder” refers to a disorder that is caused or affected by the level of activity of CBP.

As used herein, the term “EP300 loss of function mutation” refers to a mutation in EP300 that leads to the protein having diminished activity (e.g., at least 1% reduction in EP300 activity, for example 2%, 5%, 10%, 25%, 50%, or 100% reduction in CBP activity). Exemplary EP300 loss of function mutations include, but are not limited to, a homozygous EP300 mutation and chromosomal translocations.

As used herein, the term “EP300 loss of function disorder” refers to a disorder (e.g., cancer) that exhibits a reduction in EP300 activity (e.g., at least 1% reduction in EP300 activity, for example 2%, 5%, 10%, 25%, 50%, or 100% reduction in EP300 activity). By “reducing the activity of EP300” is meant reducing the level of an activity related to EP300, or a related downstream effect. The activity level of a EP300 may be measured using any method known in the art, e.g., HiBit assay.

By “reducing the level of EP300,” is meant decreasing the level of EP300 in a cell or subject. The level of EP300 may be measured using any method known in the art.

As used herein, the term “inhibitor” refers to any agent which reduces the level and/or activity of a protein (e.g., BRD9). Non-limiting examples of inhibitors include small molecule inhibitors, degraders, antibodies, enzymes, or polynucleotides (e.g., siRNA).

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, “metastatic nodule” refers to an aggregation of tumor cells in the body at a site other than the site of the original tumor.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

As used herein, the term “non-canonical BAF complex” refers to the BRG1- or HRBM-associated factors complex in a human cell that includes BRD9.

As used herein, the term “non-canonical BAF complex-related disorder” refers to a disorder that is caused or affected by the level and/or activity of a non-canonical BAF complex.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)
where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid. The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By “reducing the activity of BRD9,” is meant decreasing the level of an activity related to an BRD9, or a related downstream effect. A non-limiting example of inhibition of an activity of BRD9 is decreasing the level of a BAF complex (e.g., GBAF) in a cell. The activity level of BRD9 may be measured using any method known in the art. In some embodiments, an agent which reduces the activity of BRD9 is a small molecule BRD9 inhibitor. In some embodiments, an agent which reduces the activity of BRD9 is a small molecule BRD9 degrader.

By “reducing the level of BRD9,” is meant decreasing the level of BRD9 in a cell or subject. The level of BRD9 may be measured using any method known in the art.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a compound of the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a graph illustrating mean plasma concentration of Compound A over time after administration of various nanosuspension and microsuspension formulations tested in a rat pharmacokinetics study.

FIG. 2 is a graph illustrating mean plasma concentration of Compound A over time after administration of various nanosuspension formulations tested in a mouse pharmacokinetics study.

FIG. 3A is a graph illustrating body weight of mice after administration of various nanosuspension formulations in a rat pharmacokinetics study.

FIG. 3B is a graph illustrating tumor volume of tumors in mice after administration of various nanosuspension formulations in a rat pharmacokinetics study.

FIG. 4 is a graph illustrating mean plasma concentration of Compound A over time after administration of an amorphous microsuspension formulation tested in a rat pharmacokinetics study.

### DETAILED DESCRIPTION

The present disclosure features compositions and methods useful for the treatment of BAF-related disorders (e.g., cancer). The disclosure further features compositions and methods useful for inhibition of the level and/or activity of a protein, e.g., for the treatment of disorders such as cancer in a subject in need thereof.

### Compounds

The present disclosure provides compounds that reduce the level of an activity related to a protein, or a related downstream effect, or reduce the level of a protein in a cell or subject.

The present disclosure provides compounds that reduce the level of an activity related to BRD9, CBP, EP300, estrogen receptor, androgen receptor, IRAK4, IRAK4 and/or IMid, STAT3, MDM2, BCLXL, BTK, BTK and/or IMiD, TRK, BRM, IKZF1 and/or IKZF3, BRAF V600X, or EGFR L585R or a related downstream effect, or reduce the level of BRD9, estrogen receptor, androgen receptor, IRAK4, IRAK4 and/or IMid, STAT3, MDM2, BCLXL, BTK, BTK and/or IMiD, TRK, BRM, IKZF1 and/or IKZF3, BRAF V600X, or EGFR L585R in a cell or subject.

In some embodiments of any of the aspects described herein, the compound is compound 172 described in US Patent application publication US20220098194A1, or a pharmaceutically acceptable salt thereof. The compounds thereof are incorporated by reference herein.

In some embodiments of any of the aspects described herein, the compound is compound 176 described in US Patent application publication US20220098194A1, or a pharmaceutically acceptable salt thereof. The compounds thereof are incorporated by reference herein.

In some embodiments of any of the aspects described herein, the compound is compound 341 described in U.S. Pat. No. 10,647,698B2, or a pharmaceutically acceptable salt thereof. The compounds thereof are incorporated by reference herein.

In some embodiments of any of the aspects described herein, the compound is compound 406 described in U.S. Pat. No. 10,584,101 B2, or a pharmaceutically acceptable salt thereof. The compounds thereof are incorporated by reference herein.

In some embodiments of any of the aspects described herein, the compound is compound 483 as described in U.S. Pat. No. 11,352,350B2, or a pharmaceutically acceptable salt thereof. The compounds thereof are incorporated by reference herein.

In some embodiments of any of the aspects described herein, the compound is compound A described in US Patent application publication US20220054453A1, or a pharmaceutically acceptable salt thereof. The compounds thereof are incorporated by reference herein.

In some embodiments of any of the aspects described herein, the compound is compound 123 described in U.S. Pat. No. 11,407,732B1, or a pharmaceutically acceptable salt thereof. The compounds thereof are incorporated by reference herein.

In some embodiments of any of the aspects described herein, the compound is compound of Formula II described in U.S. Pat. No. 10,807,977B2 (¶0086), or a pharmaceutically acceptable salt thereof. The compounds thereof are incorporated by reference herein.

In some embodiments of any of the aspects described herein, the compound is compound 71 in US Patent application publication US20170065719, or a pharmaceutically acceptable salt thereof. The compounds thereof are incorporated by reference herein. These compounds can be synthesized and formulated for the treatment of cancer according to the procedure disclosed in US patent application publication US20170065719.

In some embodiments of any of the aspects described herein, the compound is a compound described in U.S. Pat. No. 10,647,698B2, or a pharmaceutically acceptable salt thereof. The compounds thereof are incorporated by reference herein. These compounds can be synthesized and formulated for the treatment of cancer according to the procedure disclosed in U.S. Pat. No. 10,647,698B2.

In some embodiments of any of the aspects described herein, the compound is a compound described in U.S. Pat. No. 10,584,101 B2, or a pharmaceutically acceptable salt thereof. The compounds thereof are incorporated by reference herein. These compounds can be synthesized and formulated for the treatment of cancer according to the procedure disclosed in U.S. Pat. No. 10,584,101 B2.

In some embodiments of any of the aspects described herein, the compound is a compound described in US Patent US U.S. Ser. No. 11/352,350B2, or a pharmaceutically acceptable salt thereof. The compounds thereof are incorporated by reference herein. These compounds can be synthesized and formulated for the treatment of cancer according to the procedure disclosed in U.S. Pat. No. 11,352,350B2.

In some embodiments of any of the aspects described herein, the compound is a compound described in US Patent application publication US20220054453A1, or a pharmaceutically acceptable salt thereof. The compounds thereof are incorporated by reference herein. These compounds can be synthesized and formulated for the treatment of cancer according to the procedure disclosed in US patent application publication US20220054453A1.

In some embodiments of any of the aspects described herein, the compound is a compound described in US Patent application publication US20190076540A1, or a pharmaceutically acceptable salt thereof. The compounds thereof are incorporated by reference herein. These compounds can be synthesized and formulated for the treatment of cancer according to the procedure disclosed in US patent application publication US20190076540A1.

In some embodiments of any of the aspects described herein, the compound is a compound described in U.S. Pat. No. 10,660,968B2, or a pharmaceutically acceptable salt thereof. The compounds thereof are incorporated by reference herein. These compounds can be synthesized and formulated for the treatment of cancer according to the procedure disclosed in U.S. Pat. No. 10,660,968B2.

In some embodiments of any of the aspects described herein, the compound is a compound described in US Patent application publication US20210198256A1, or a pharmaceutically acceptable salt thereof. The compounds thereof are incorporated by reference herein. These compounds can be synthesized and formulated for the treatment of cancer according to the procedure disclosed in US patent application publication US20210198256A1.

In some embodiments of any of the aspects described herein, the compound is a compound described in US Patent application publication US20190076542A1, or a pharmaceutically acceptable salt thereof. The compounds thereof are incorporated by reference herein. These compounds can be synthesized and formulated for the treatment of cancer according to the procedure disclosed in US patent application publication US20190076542A1.

In some embodiments of any of the aspects described herein, the compound is a compound described in U.S. Pat. No. 10,849,982B2, or a pharmaceutically acceptable salt thereof. The compounds thereof are incorporated by reference herein. These compounds can be synthesized and formulated for the treatment of cancer according to the procedure disclosed in U.S. Pat. No. 10,849,982B2.

In some embodiments of any of the aspects described herein, the compound is a compound described in US Patent application publication US20220313827A1, or a pharmaceutically acceptable salt thereof. The compounds thereof are incorporated by reference herein. These compounds can be synthesized and formulated for the treatment of cancer according to the procedure disclosed in US patent application publication US20220313827A1.

In some embodiments of any of the aspects described herein, the compound is a compound described in US Patent application publication US20220372016A1, or a pharmaceutically acceptable salt thereof. The compounds thereof are incorporated by reference herein. These compounds can be synthesized and formulated for the treatment of cancer according to the procedure disclosed in US patent application publication US20220372016A1.

In some embodiments of any of the aspects described herein, the compound is a compound described in U.S. Pat. No. 11,524,949B2, or a pharmaceutically acceptable salt thereof. The compounds thereof are incorporated by reference herein. These compounds can be synthesized and formulated for the treatment of cancer according to the procedure disclosed in U.S. Pat. No. 11,524,949B2.

In some embodiments of any of the aspects described herein, the compound is a compound described in US Patent application publication US20200140456A, or a pharmaceutically acceptable salt thereof. The compounds thereof are incorporated by reference herein. These compounds can be synthesized and formulated for the treatment of cancer according to the procedure disclosed in US patent application publication US20200140456A.

In some embodiments of any of the aspects described herein, the compound is a compound described in U.S. Pat. No. 11,459,335B2, or a pharmaceutically acceptable salt thereof. The compounds thereof are incorporated by reference herein. These compounds can be synthesized and formulated for the treatment of cancer according to the procedure disclosed in U.S. Pat. No. 11,459,335B2.

In some embodiments of any of the aspects described herein, the compound is a compound described in US Patent application publication US20210238193A1, or a pharmaceutically acceptable salt thereof. The compounds thereof are incorporated by reference herein. These compounds can be synthesized and formulated for the treatment of cancer according to the procedure disclosed in US patent application publication US20210238193A1.

In some embodiments of any of the aspects described herein, the compound is a compound described in US Patent application publication US20200377469A1, or a pharmaceutically acceptable salt thereof. The compounds thereof are incorporated by reference herein. These compounds can be synthesized and formulated for the treatment of cancer according to the procedure disclosed in US patent application publication US20200377469A1.

In some embodiments of any of the aspects described herein, the compound is a compound described in U.S. Pat. No. 11,358,948B2, or a pharmaceutically acceptable salt thereof. The compounds thereof are incorporated by reference herein. These compounds can be synthesized and formulated for the treatment of cancer according to the procedure disclosed in U.S. Pat. No. 11,358,948B2.

In some embodiments of any of the aspects described herein, the compound is a compound described in US Patent application publication US20210002296A1, or a pharmaceutically acceptable salt thereof. The compounds thereof are incorporated by reference herein. These compounds can be synthesized and formulated for the treatment of cancer according to the procedure disclosed in US patent application publication US20210002296A1.

In some embodiments of any of the aspects described herein, the compound is a compound described in US Patent application publication US20200347045A1, or a pharmaceutically acceptable salt thereof. The compounds thereof are incorporated by reference herein. These compounds can be synthesized and formulated for the treatment of cancer according to the procedure disclosed in US patent application publication US20200347045A1.

In some embodiments of any of the aspects described herein, the compound is a compound described in U.S. Pat. No. 11,512,080B2, or a pharmaceutically acceptable salt thereof. The compounds thereof are incorporated by reference herein. These compounds can be synthesized and formulated for the treatment of cancer according to the procedure disclosed in U.S. Pat. No. 11,512,080B2.

In some embodiments of any of the aspects described herein, the compound is a compound described in US Patent application publication US20200010468A1, or a pharmaceutically acceptable salt thereof. The compounds thereof are incorporated by reference herein. These compounds can be synthesized and formulated for the treatment of cancer according to the procedure disclosed in US patent application publication US20200010468A1.

In some embodiments of any of the aspects described herein, the compound is a compound described in U.S. Pat. No. 11,292,792B2, or a pharmaceutically acceptable salt thereof. The compounds thereof are incorporated by reference herein. These compounds can be synthesized and formulated for the treatment of cancer according to the procedure disclosed in U.S. Pat. No. 11,292,792B2.

In some embodiments of any of the aspects described herein, the compound is a compound described in US Patent application publication US20220348556A1, or a pharmaceutically acceptable salt thereof. The compounds thereof are incorporated by reference herein. These compounds can be synthesized and formulated for the treatment of cancer according to the procedure disclosed in US patent application publication US20220348556A1.

In some embodiments of any of the aspects described herein, the compound is a compound described in US Patent application publication US20220281831A1, or a pharmaceutically acceptable salt thereof. The compounds thereof are incorporated by reference herein. These compounds can be synthesized and formulated for the treatment of cancer according to the procedure disclosed in US patent application publication US20220281831A1.

In some embodiments of any of the aspects described herein, the compound is a compound described in US Patent application publication US20220331317A1, or a pharmaceutically acceptable salt thereof. The compounds thereof are incorporated by reference herein. These compounds can be synthesized and formulated for the treatment of cancer according to the procedure disclosed in US patent application publication US20220331317A1.

In some embodiments of any of the aspects described herein, the compound is a compound described in US Patent application publication US20180044335A1, or a pharmaceutically acceptable salt thereof. The compounds thereof are incorporated by reference herein. These compounds can be synthesized and formulated for the treatment of cancer according to the procedure disclosed in US patent application publication US20180044335A1.

In some embodiments of any of the aspects described herein, the compound is a compound described in U.S. Pat. No. 11,319,318B2, or a pharmaceutically acceptable salt thereof. The compounds thereof are incorporated by reference herein. These compounds can be synthesized and formulated for the treatment of cancer according to the procedure disclosed in U.S. Pat. No. 11,319,318B2.

In some embodiments of any of the aspects described herein, the compound is a compound described in US Patent application publication US20210380579A1, or a pharmaceutically acceptable salt thereof. The compounds thereof are incorporated by reference herein. These compounds can be synthesized and formulated for the treatment of cancer according to the procedure disclosed in US patent application publication US20210380579A1.

In some embodiments of any of the aspects described herein, the compound is a compound described in US Patent application publication US20220387602A1, or a pharmaceutically acceptable salt thereof. The compounds thereof are incorporated by reference herein. These compounds can be synthesized and formulated for the treatment of cancer according to the procedure disclosed in US patent application publication US20220387602A1.

In some embodiments of any of the aspects described herein, the compound is a compound described in US Patent application publication US20210323956A1, or a pharmaceutically acceptable salt thereof. The compounds thereof are incorporated by reference herein. These compounds can be synthesized and formulated for the treatment of cancer according to the procedure disclosed in US patent application publication US20210323956A1.

In some embodiments of any of the aspects described herein, the compound is a compound described in US Patent application publication US20220315578A1, or a pharmaceutically acceptable salt thereof. The compounds thereof are incorporated by reference herein. These compounds can be synthesized and formulated for the treatment of cancer according to the procedure disclosed in US patent application publication US20220315578A1.

In some embodiments of any of the aspects described herein, the compound is a compound described in US Patent application publication US20210155932A1, or a pharmaceutically acceptable salt thereof. The compounds thereof are incorporated by reference herein. These compounds can be synthesized and formulated for the treatment of cancer according to the procedure disclosed in US patent application publication US20210155932A1.

In some embodiments of any of the aspects described herein, the compound is a compound described in U.S. Pat. No. 11,530,413B2, or a pharmaceutically acceptable salt thereof. The compounds thereof are incorporated by reference herein. These compounds can be synthesized and formulated for the treatment of cancer according to the procedure disclosed in U.S. Pat. No. 11,530,413B2.

In some embodiments of any of the aspects described herein, the compound is a compound described in US Patent application publication US20160032402A1, or a pharmaceutically acceptable salt thereof. The compounds thereof are incorporated by reference herein. These compounds can be synthesized and formulated for the treatment of cancer according to the procedure disclosed in US patent application publication US20160032402A1.

In some embodiments of any of the aspects described herein, the compound is a compound described in US Patent U.S. Pat. No. 9,850,543B2, or a pharmaceutically acceptable salt thereof. The compounds thereof are incorporated by reference herein. These compounds can be synthesized and formulated for the treatment of cancer according to the procedure disclosed in US patent U.S. Pat. No. 9,850,543B2.

In some embodiments of any of the aspects described herein, the compound is a compound described in US Patent application publication US20220033409A1, or a pharmaceutically acceptable salt thereof. The compounds thereof are incorporated by reference herein. These compounds can be synthesized and formulated for the treatment of cancer according to the procedure disclosed in US patent application publication US20220033409A1.

In some embodiments of any of the aspects described herein, the compound is a compound described in US Patent application publication US20190300521A1, or a pharmaceutically acceptable salt thereof. The compounds thereof are incorporated by reference herein. These compounds can be synthesized and formulated for the treatment of cancer according to the procedure disclosed in US patent application publication US20190300521A1.

In some embodiments of any of the aspects described herein, the compound is a compound described in PCT application publication WO2022042707A1A1, or a pharmaceutically acceptable salt thereof. The compounds thereof are incorporated by reference herein. These compounds can be synthesized and formulated for the treatment of cancer according to the procedure disclosed in PCT application publication WO2022042707A1.

In some embodiments of any of the aspects described herein, the compound is a compound described in US Patent application publication US2023073777A1, or a pharmaceutically acceptable salt thereof. The compounds thereof are incorporated by reference herein. These compounds can be synthesized and formulated for the treatment of cancer according to the procedure disclosed in US patent application publication US2023073777A1.

In some embodiments of any of the aspects described herein, the compound is a compound described in US Patent application publication US2023027088A1, or a pharmaceutically acceptable salt thereof. The compounds thereof are incorporated by reference herein. These compounds can be synthesized and formulated for the treatment of cancer according to the procedure disclosed in US patent application publication US2023027088A1.

In some embodiments of any of the aspects described herein, the compound is a compound described in US Patent application publication US2023322724A1, or a pharmaceutically acceptable salt thereof. The compounds thereof are incorporated by reference herein. These compounds can be synthesized and formulated for the treatment of cancer according to the procedure disclosed in US patent application publication US2023322724A1.

In some embodiments of any of the aspects described herein, the compound is a compound described in PCT application publication WO2022187417A1, or a pharmaceutically acceptable salt thereof. The compounds thereof are incorporated by reference herein. These compounds can be synthesized and formulated for the treatment of cancer according to the procedure disclosed in PCT application publication WO2022187417A1.

In some embodiments of any of the aspects described herein, the compound is a compound described in U.S. Pat. No. 10,696,655B2, or a pharmaceutically acceptable salt thereof. The compounds thereof are incorporated by reference herein. These compounds can be synthesized and formulated for the treatment of cancer according to the procedure disclosed in U.S. Pat. No. 10,696,655B2.

In some embodiments of any of the aspects described herein, the compound is a compound described in U.S. Pat. No. 11,168,070B2, or a pharmaceutically acceptable salt thereof. The compounds thereof are incorporated by reference herein. These compounds can be synthesized and formulated for the treatment of cancer according to the procedure disclosed in U.S. Pat. No. 11,168,070B2.

In some embodiments of any of the aspects described herein, the compound is a compound described in U.S. Pat. No. 11,247,989B2, or a pharmaceutically acceptable salt thereof. The compounds thereof are incorporated by reference herein. These compounds can be synthesized and formulated for the treatment of cancer according to the procedure disclosed in U.S. Pat. No. 11,247,989B2.

In some embodiments of any of the aspects described herein, the compound is a compound described in US Patent application publication US2022127265A1, or a pharmaceutically acceptable salt thereof. The compounds thereof are incorporated by reference herein. These compounds can be synthesized and formulated for the treatment of cancer according to the procedure disclosed in US patent application publication US2022127265A1.

In some embodiments of any of the aspects described herein, the compound is a compound described in US Patent U.S. Pat. No. 9,763,922B2, or a pharmaceutically acceptable salt thereof. The compounds thereof are incorporated by reference herein. These compounds can be synthesized and formulated for the treatment of cancer according to the procedure disclosed in US patent U.S. Pat. No. 9,763,922B2.

In some embodiments of any of the aspects described herein, the compound is a compound described in U.S. Pat. No. 10,206,931 B2, or a pharmaceutically acceptable salt thereof. The compounds thereof are incorporated by reference herein. These compounds can be synthesized and formulated for the treatment of cancer according to the procedure disclosed in U.S. Pat. No. 10,206,931 B2.

In some embodiments of any of the aspects described herein, the compound is a compound described in US Patent application publication US2017196878A1, or a pharmaceutically acceptable salt thereof. The compounds thereof are incorporated by reference herein. These compounds can be synthesized and formulated for the treatment of cancer according to the procedure disclosed in US patent application publication US2017196878A1.

In some embodiments of any of the aspects described herein, the compound is a compound described in U.S. Pat. No. 10,358,437B2, or a pharmaceutically acceptable salt thereof. The compounds thereof are incorporated by reference herein. These compounds can be synthesized and formulated for the treatment of cancer according to the procedure disclosed in U.S. Pat. No. 10,358,437B2.

In some embodiments of any of the aspects described herein, the compound is a compound described in US Patent application publication US2016317632A1, or a pharmaceutically acceptable salt thereof. The compounds thereof are incorporated by reference herein. These compounds can be synthesized and formulated for the treatment of cancer according to the procedure disclosed in US patent application publication US2016317632A1.

In some embodiments of any of the aspects described herein, the compound is a compound described in U.S. Pat. No. 10,428,065B2, or a pharmaceutically acceptable salt thereof. The compounds thereof are incorporated by reference herein. These compounds can be synthesized and formulated for the treatment of cancer according to the procedure disclosed in U.S. Pat. No. 10,428,065B2.

In some embodiments of any of the aspects described herein, the compound is a compound described in U.S. Pat. No. 10,118,920B2, or a pharmaceutically acceptable salt thereof. The compounds thereof are incorporated by reference herein. These compounds can be synthesized and formulated for the treatment of cancer according to the procedure disclosed in U.S. Pat. No. 10,118,920B2.

In some embodiments of any of the aspects described herein, the compound is a compound described in U.S. Pat. No. 10,696,666B2, or a pharmaceutically acceptable salt thereof. The compounds thereof are incorporated by reference herein. These compounds can be synthesized and formulated for the treatment of cancer according to the procedure disclosed in U.S. Pat. No. 10,696,666B2.

In some embodiments of any of the aspects described herein, the compound is a compound described in U.S. Pat. No. 10,647,704B2, or a pharmaceutically acceptable salt thereof. The compounds thereof are incorporated by reference herein. These compounds can be synthesized and formulated for the treatment of cancer according to the procedure disclosed in U.S. Pat. No. 10,647,704B2.

In some embodiments of any of the aspects described herein, the compound is a compound described in U.S. Pat. No. 11,453,662B2, or a pharmaceutically acceptable salt thereof. The compounds thereof are incorporated by reference herein. These compounds can be synthesized and formulated for the treatment of cancer according to the procedure disclosed in U.S. Pat. No. 11,453,662B2. For example, the compound may be any one of compounds 1-9 or a pharmaceutically acceptable salt thereof. For example, the compound may be:

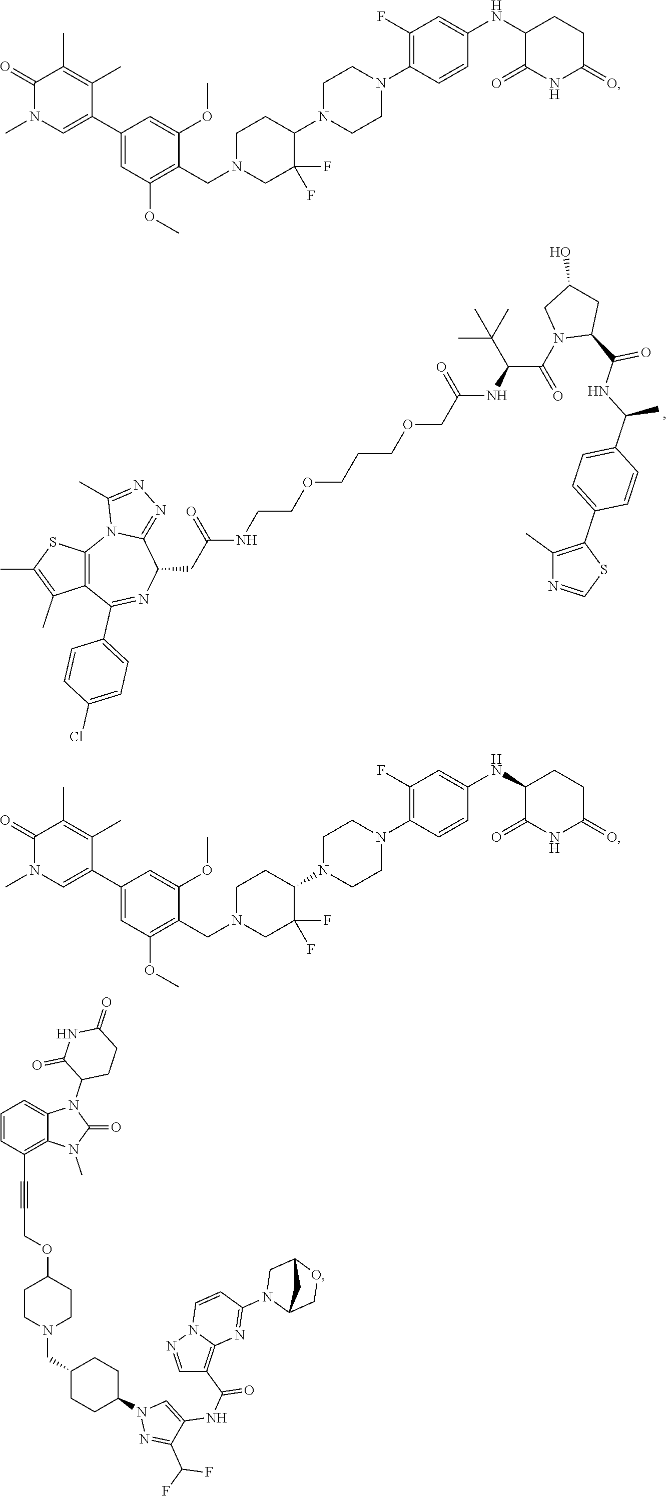 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1 ; Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1 ; COc1cc(-c2cn(C)c(=O)c(C)c2C)cc(OC)c1CN1CC[C@H](N2CCN(c3ccc(N[C@H]4CCC(=O)NC4=O)cc3F)CC2)C(F)(F)C1 ; Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

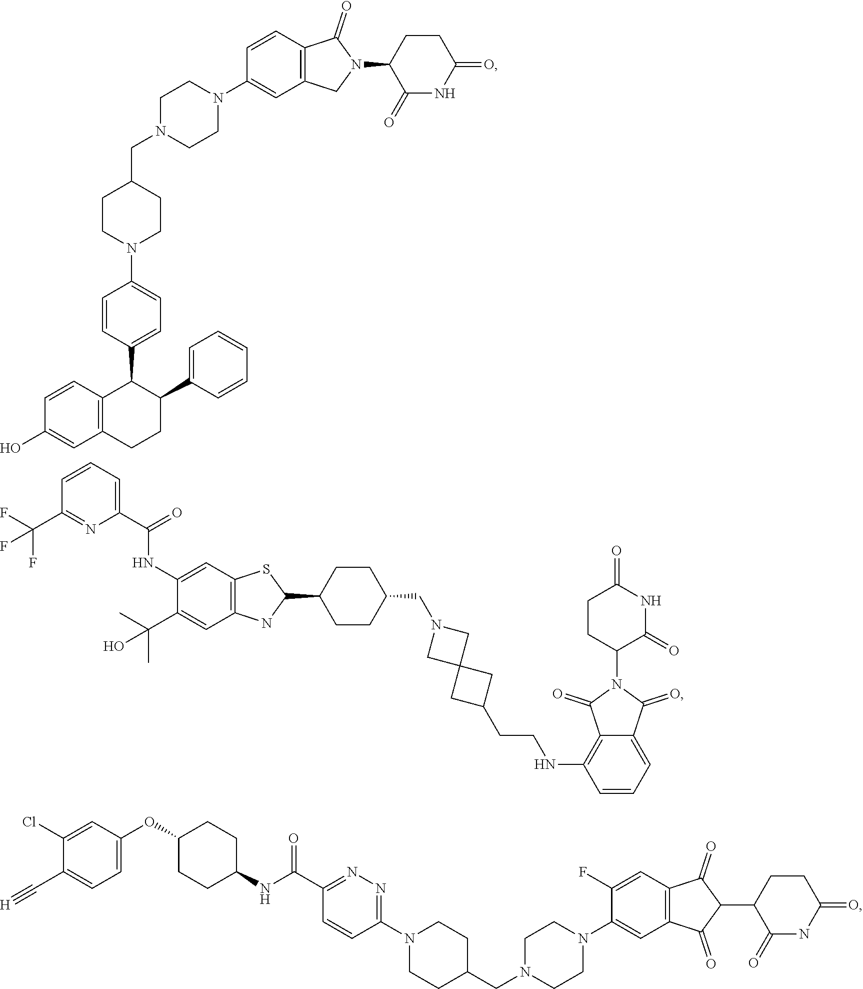

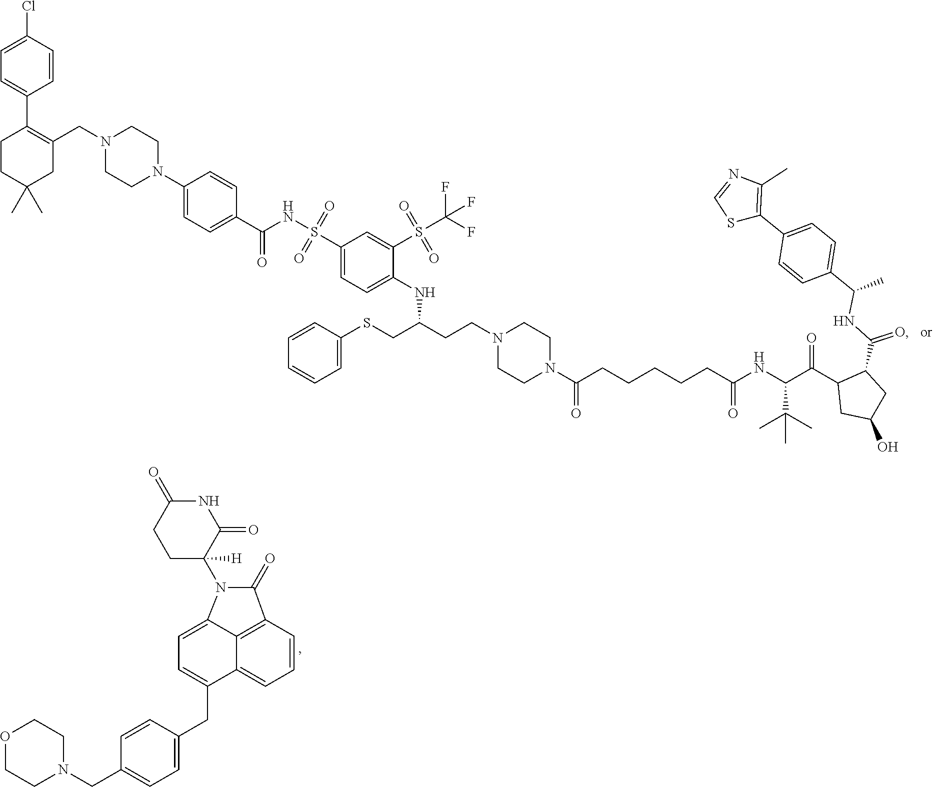 [SMILES: Cc1ncsc1-c1ccc([C@H](C)NC(=O)[C@@H]2C[C@@H](O)CC2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1 ; O=C1CC[C@H](N2C(=O)c3cccc4c(Cc5ccc(CN6CCOCC6)cc5)ccc2c34)C(=O)N1]

or a pharmaceutically acceptable salt thereof.

In some embodiments of any of the aspects described herein, the compound is a compound described in US Patent US20220098194A1, or a pharmaceutically acceptable salt thereof. For example, the compound is of the following structure:

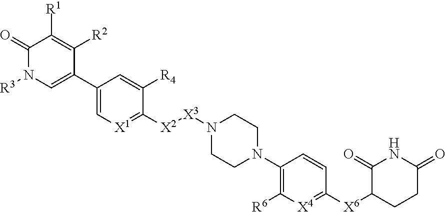

R¹ is NH₂, or CH₃;
R² is H, or CH₃;
R³ is H, CH₃ or OCH₃;
R⁴ is H, CH₃, OCH₃, CF₂H, or CF₃;
R5 is H, OH, CH₃, CFH2, F, Cl, Br, or I;
X¹ is CH, N, COCH₃, or CCF₃;
X² is CH₂, C(O), or O;
X³ is heterocycle optionally substituted with 1, 2, or 3 groups independently selected from RX;
X⁴ is N, CH, or CCH₃;
X⁵ is NH, NCH₃, CH₂, O, and S;

In some embodiments of any of the aspects described herein, the compound is a compound described in U.S. Pat. No. 10,647,698B2, or a pharmaceutically acceptable salt thereof. For example, the compound is of the following structure:

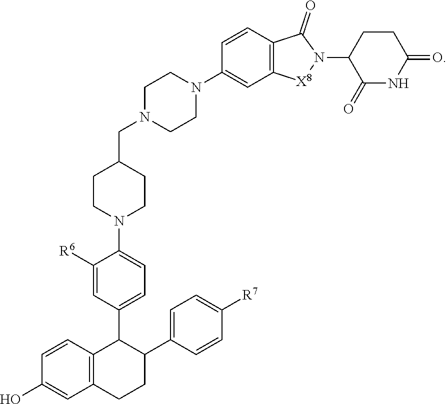

R⁶ is H, F or CH₃,
R⁷ is H, CH₃, OCH₃, or OH;
X⁶ is CH₂, or C(O).

In some embodiments of any of the aspects described herein, the compound is a compound described in U.S. Pat. No. 10,584,101 B2, or a pharmaceutically acceptable salt thereof. For example, the compound is of the following structure:

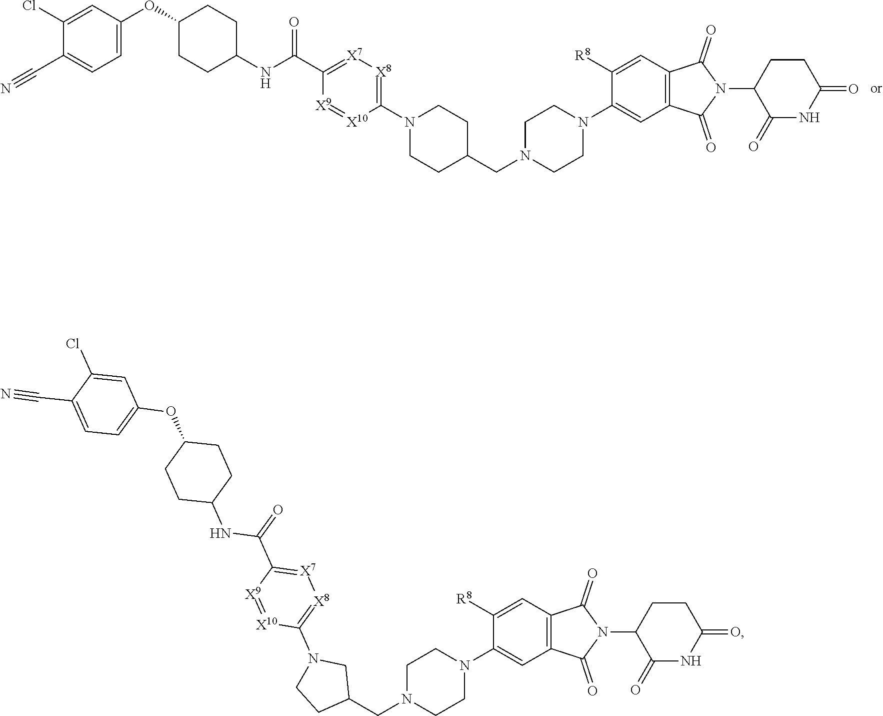

R⁸ is H or F;
X⁷ is CH₂ or N;
X⁸ is CH₂ or N;
X⁹ is CH₂ or N;
X¹⁰ is CH₂ or N.

In some embodiments of any of the aspects described herein, the compound is a compound described in U.S. Pat. No. 11,407,732B1, or a pharmaceutically acceptable salt thereof. For example, the compound is of the following formula:

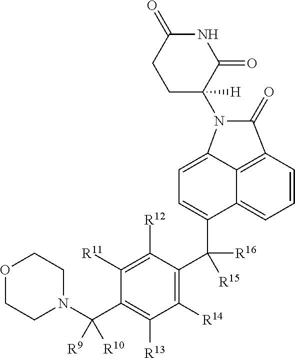

R⁹ is H, or CH₃;
R¹⁰ is H, or CH₃;
R¹¹ is H, or CH₃;
R¹² is H, or CH₃;
R¹³ is H, or CH₃;
R¹⁴ is H, or CH₃;
R¹⁵ is H, or CH₃;
R¹⁶ is H, or CH₃.

In some embodiments of any of the aspects described herein, the compound is a compound described in US Patent US20170065719, or a pharmaceutically acceptable salt thereof. For example, the compound is of the following formula:

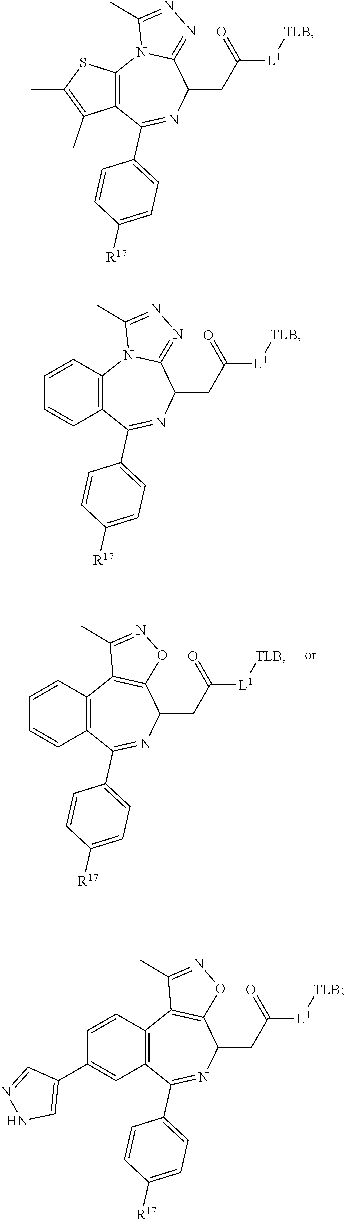

R¹⁷ is H, Cl, F, Br, CN, CH3, OCH3, or C(O)CH3;

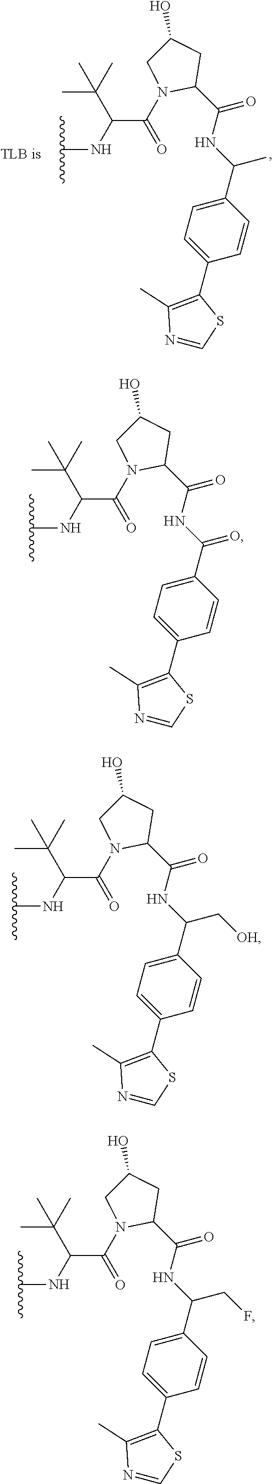

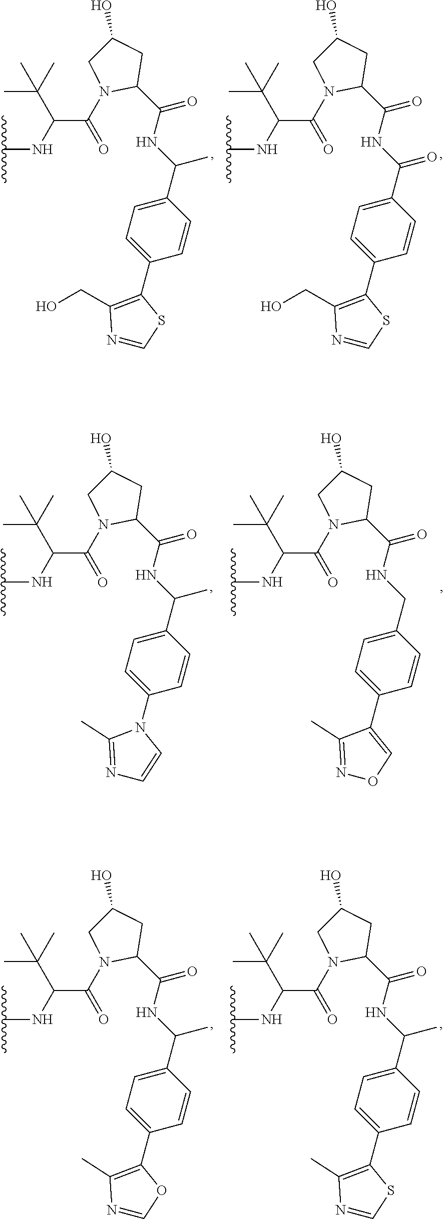

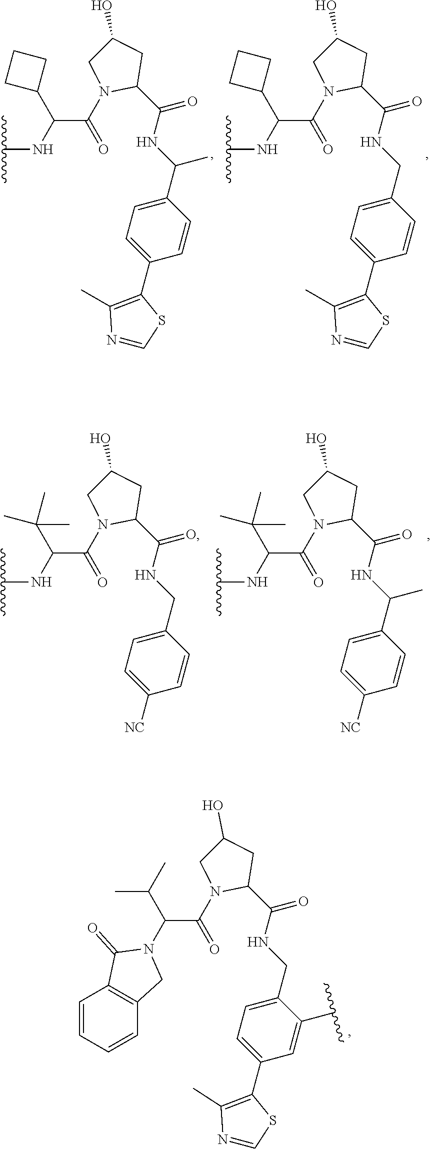

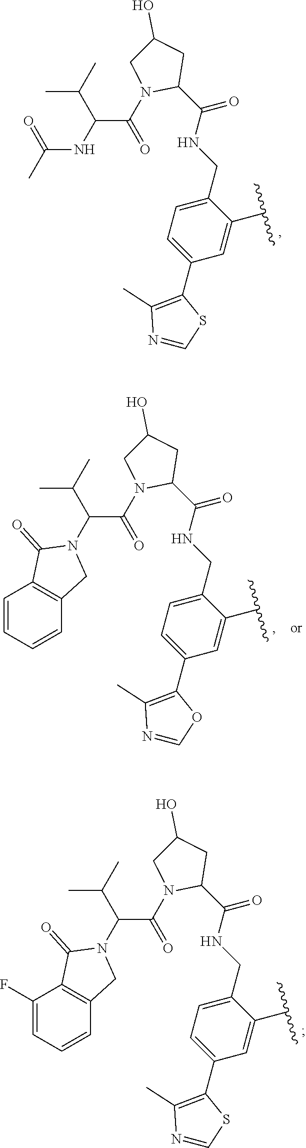 [SMILES: *c1cc(-c2scnc2C)ccc1CNC(=O)C1CC(O)CN1C(=O)C(NC(C)=O)C(C)C ; *c1cc(-c2ocnc2C)ccc1CNC(=O)C1CC(O)CN1C(=O)C(C(C)C)N1Cc2ccccc2C1=O ; *c1cc(-c2scnc2C)ccc1CNC(=O)C1CC(O)CN1C(=O)C(C(C)C)N1Cc2cccc(F)c2C1=O]

L¹ is —N(R)—(CH2)ₘ-O(CH2)ₙ-O(CH2)ₒ-O(CH2)ₚ-O(CH2)_{q}-O(CH2)ᵣOCH2-, —O—(CH2)ₘ—O(CH2)ₙ—O(CH2)ₒ-O(CH2)ₚ—O(CH2)_{q}—O(CH2)ᵣ-OCH2-, —O—(CH2)ₘ-O(CH2)ₙ-O(CH2)ₒ-O(CH2)ₚ-O(CH2)_{q}-O(CH2)ᵣ-O—, —N(R)—(CH2)ₘ-O(CH2)ₙ-O(CH2)ₒ-O(CH2)ₚ-O(CH2)_{q}—O(CH2)ᵣ-O—, —(CH2)ₘ-O(CH2)ₙ-O(CH2)ₒ-O(CH2)ₚ-O(CH2)_{q}-O(CH2)ᵣ-O—, —(CH2)ₘ-O(CH2)ₙ-O(CH2)ₒ-O(CH2)ₚ-O(CH2)_{q}-O(CH2)ᵣ-OCH2-,

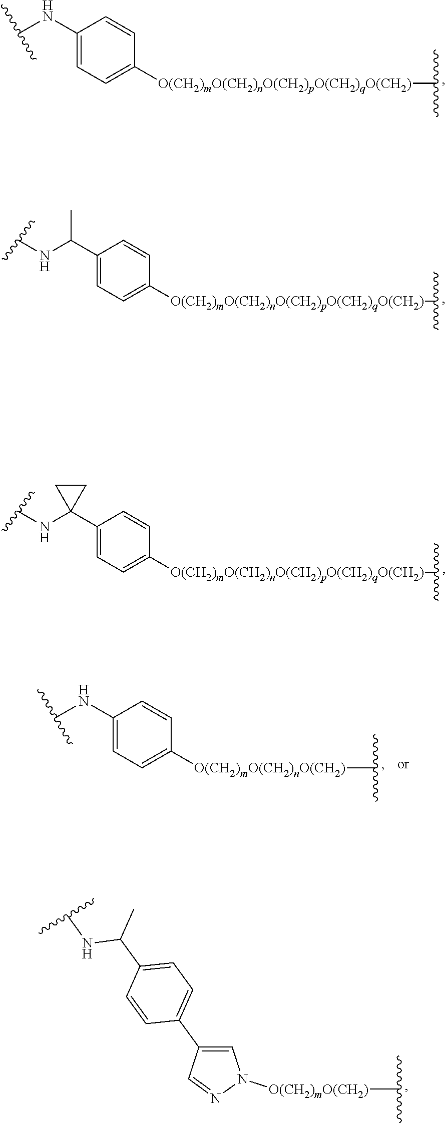

Wherein m, n, o, p, q and r are 1,2,3, or 4.

In some embodiments of any of the aspects described herein, the compound is a compound described in U.S. Pat. No. 11,352,350B2, or a pharmaceutically acceptable salt thereof. For example, the compound is of the following formula:

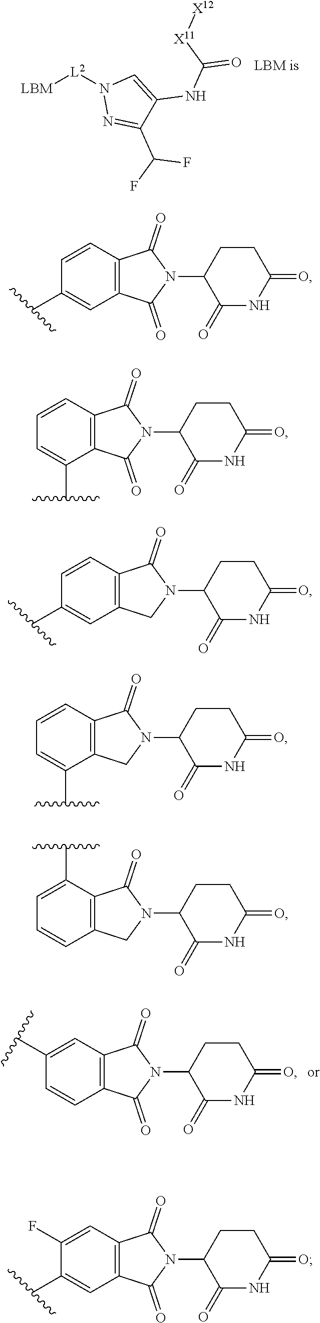

L² is an optionally substituted C₁-C₁₀ alkyl, C₁₋₁₀ heteroalkyl, C₁-C₁₀ alkynyl, 3-12 membered carbocycle, or 3-12 membered heterocycle, wherein each C₁₋₁₀ alkyl, C₁-C₁₀ alkynyl, 3-12 membered carbocycle, or 3-12 membered heterocycle of L² is optionally substituted with LBM, and/or one or more groups of R^{A10},
Wherein R^{A10} is C₁-C₁₀ alkyl, C₁-C₁₀ alkynyl, 3-12 membered carbocycle, or 3-12 membered heterocycle is independently optionally substituted with LBM, and/or one or more of the substituent groups selected from CH₃, OH, OCH₃ and F.
X¹¹ is

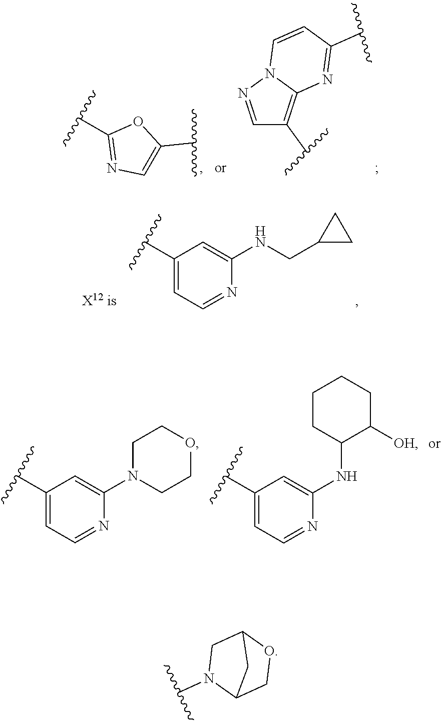

In some embodiments of any of the aspects described herein, the compound is a compound described in US Patent US20220054453A1, or a pharmaceutically acceptable salt thereof. For example, the compound is N-(2-((1r,4r)-4-((6-(2-((2-(2,6-dioxopiperidin-3-yl)-1,3-dioxoisoindolin-4-yl)amino)ethyl)-2-azaspiro[3.3]heptan-2-yl)methyl)cyclohexyl)-5-(2-hydroxypropan-2-yl)benzo[d]thiazol-6-yl)-6-(trifluoromethyl)picolinamide.

In some embodiments of any of the aspects described herein, the compound is a compound described in U.S. Pat. No. 11,352,350B2, or a pharmaceutically acceptable salt thereof. For example, the compound is of the following formula:

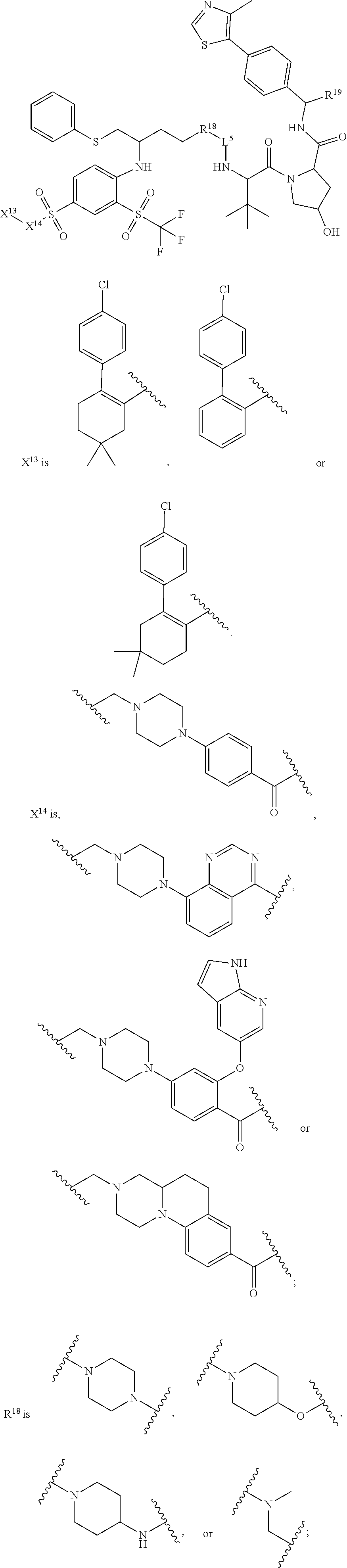

R¹⁹ is H or CH₃;
L³ is C(O)—(CH2)ₘ-O(CH2)ₙ-O(CH2)ₒ-O(CH2)ₚ-O(CH2)_{q}-O(CH2)ᵣ-OCH2-C(O), C(O)—(CH2)ₘ-O(CH2)ₙ-O(CH2)ₒ-O(CH2)ₚ-O(CH2)_{q}-OCH2-C(O), C(O)—(CH2)ₘ-O(CH2)ₙ-O(CH2)ₒ-O(CH2)ₚ-OCH2-C(O), C(O)—(CH2)ₘ-O(CH2)ₙ-O(CH2)ₒ-O(CH2)ᵣ-OCH2-C(O), C(O)—(CH2)ₘ-OCH2-C(O), wherein m, n, o, p, q and r are 1,2,3, or 4.

The compounds found in US Patent Nos US20220098194A1, US20170065719, U.S. Ser. No. 11/352,350B2, U.S. Ser. No. 10/647,698B2, US20220054453A1, U.S. Ser. No. 10/584,101B2, U.S. Ser. No. 10/807,977B2, and U.S. Ser. No. 11/407,732B1 are incorporated by reference herein. These compounds can be synthesized and formulated for the treatment of cancer according to the procedure disclosed in US patent Nos US20220098194A1, US20170065719, U.S. Ser. No. 11/352,350B2, U.S. Ser. No. 10/647,698B2, US20220054453A1, U.S. Ser. No. 10/584,101 B2, U.S. Ser. No. 10/807,977B2, and U.S. Ser. No. 11/407,732B1.

### Pharmaceutical Uses

The compounds described herein are useful in the methods of the disclosure and, while not bound by theory, are believed to exert their desirable effects through their ability to modulate the level, status, and/or activity of BRD9, CBP, EP300, estrogen receptor, androgen receptor, IRAK4, IRAK4 and/or IMid, STAT3, MDM2, BCLXL, BTK, BTK and/or IMiD, TRK, BRM, IKZF1 and/or IKZF3, BRAF V600X, or EGFR L585R protein in a cell in a mammal.

An aspect of the present disclosure relates to methods of treating disorders related to BRD9, CBP, EP300, estrogen receptor, androgen receptor, IRAK4, IRAK4 and/or IMid, STAT3, MDM2, BCLXL, BTK, BTK and/or IMiD, TRK, BRM, IKZF1 and/or IKZF3, BRAF V600X, or EGFR L585R such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f reduced rate of metastasis, (g) decreased tumor recurrence (h) increased survival of subject, and (i) increased progression free survival of a subject.

An aspect of the present disclosure relates to methods of treating disorders related to BRD9 such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

In some embodiments of any of the aspects described herein, the cancer is a malignant rhabdoid tumor, a CDS+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma, non-small cell lung cancer, stomach cancer, breast cancer, or SMARCAB1-loss cancer.

In some embodiments, the cancer is a malignant rhabdoid tumor.

In some embodiments, the cancer is a CDS+ T-cell lymphoma.

In some embodiments, the cancer is endometrial carcinoma.

In some embodiments, the cancer is ovarian carcinoma.

In some embodiments, the cancer is bladder cancer.

In some embodiments, the cancer is stomach cancer.

In some embodiments, the cancer is pancreatic cancer.

In some embodiments, the cancer is esophageal cancer.

In some embodiments, the cancer is prostate cancer.

In some embodiments, the cancer is renal cell carcinoma.

In some embodiments, the cancer is melanoma.

In some embodiments, the cancer is colorectal cancer.

In some embodiments, the cancer is a sarcoma, e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, or low-grade rhabdomyosarcoma. In particular embodiments, the sarcoma is synovial sarcoma.

In some embodiments, the cancer is non-small cell lung cancer.

In some embodiments, the cancer is breast cancer.

In some embodiments, the cancer is a SMARCAB1-loss cancer, e.g., high grade ovarian serous adenocarcinoma, epithelioid sarcoma, atypical teratoid/rhabdoid tumor, malignant rhabdoid tumor (MRT), prostate adenocarcinoma, conventional glioblastoma multiforme, breast invasive ductal carcinoma, sarcoma (NOS), gastrointestinal stromal tumor, kidney/renal medullary carcinoma, pancreatic neuroendocrine neoplasm, sinonasal adenocarcinoma, adrenal cortex carcinoma, proximal-type epithelioid sarcoma, head and neck carcinoma, invasive breast carcinoma, rhabdoid tumor of the kidney, dedifferentiated liposarcoma, pancreatic adenocarcinoma, chordoma, testicular yolk sac tumor, small cell lung carcinoma, squamous cell lung carcinoma, acral lentiginous melanoma, peripheral T-cell lymphoma, nasal cavity and paranasal sinus squamous cell carcinoma, myoepithelial carcinoma, malignant peripheral nerve sheath tumor, breast invasive lobular carcinoma, chordoma, a SMARCB1-mutant cancer, or lung adenocarcinoma. In particular embodiments, the SMARCAB1-loss cancer is chordoma.

In one aspect, the present disclosure provides a method of treating cancer in a subject in need thereof, the method comprising administering to the subject an effective amount of a long-acting formulation of a compound that reduces the level and/or activity of BRD9, or a pharmaceutically acceptable salt thereof.

In one aspect, the present disclosure provides a method of treating cancer in a subject in need thereof, the method comprising administering to the subject an effective amount of a long-acting formulation of any one of compounds 1-9, or a pharmaceutically acceptable salt thereof.

In some embodiments, the cancer is synovial sarcoma.

In one aspect, the present disclosure provides a method of treating synovial sarcoma in a subject in need thereof, the method comprising administering to the subject an effective amount of a long-acting formulation of any one of compounds 1-9 or a pharmaceutically acceptable salt thereof.

In one aspect, the present disclosure provides a method of treating cancer in a subject in need thereof, the method comprising administering to the subject an effective amount of a long-acting formulation of a compound that reduces the level and/or activity of CBP, or a pharmaceutically acceptable salt thereof. In some embodiments, the cancer is gastric cancer. In some embodiments, the cancer is colorectal cancer. In some embodiments, the cancer is bladder cancer.

In one aspect, the present disclosure provides a method of treating cancer in a subject in need thereof, the method comprising administering to the subject an effective amount of a long-acting formulation of a compound that reduces the level and/or activity of EP300, or a pharmaceutically acceptable salt thereof. In some embodiments, the cancer is prostate cancer.

### Long-Acting Formulations

The term “long acting” may be referred to in the art as “sustained release” and/or “extended release”.

The term “long acting” as used herein refers to a dosage formulation that provides for the release of a compound over an extended period of time. In some embodiments, the release of a compound will occur over a period of about 1 day. In some embodiments, the release of a compound will occur over a period of about 2 days. In some embodiments, the release of a compound will occur over a period of about 3 days. In some embodiments, the release of a compound will occur over a period of about 4 days. In some embodiments, the release of a compound will occur over a period of about 5 days. In some embodiments, the release of a compound will occur over a period of about 6 days. In some embodiments, the release of a compound will occur over a period of about 1 week. In some embodiments, the release of a compound will occur over a period of about 2 weeks. In some embodiments, the release of a compound will occur over a period of about 3 weeks. In some embodiments, the release of a compound will occur over a period of about 4 weeks. In some embodiments, the release of a compound will occur over a period of about 5 weeks. In some embodiments, the release of a compound will occur over a period of about 6 weeks. In some embodiments, the release of a compound will occur over a period of about 8 weeks. In some embodiments, the release of a compound will occur over a period of about 1 month. In some embodiments, the release of a compound will occur over a period of about 2 months. In some embodiments, the release of a compound will occur over a period of about 1 day to about 1 month. In some embodiments, the release of a compound will occur over a period of about 1 day to about 2 months. In some embodiments, the release of a compound will occur over a period of about 1 week to about 1 month. In some embodiments, the release of a compound will occur over a period of about 1 week to about 2 months. In some embodiments, the release of a compound will occur over a period of about 2 weeks to about 1 month. In some embodiments, the release of a compound will occur over a period of about 2 weeks to about 2 months. In some embodiments, the release of a compound will occur over a period of about 1 month to about 2 months.

In some embodiments, the term “long acting” refers to a release profile wherein the plasma concentration of the compound is maintained at steady state for an extended period of time. In some embodiments, the plasma concentration of the compound is maintained at steady state over a period of about 1 day. In some embodiments, the plasma concentration of the compound is maintained at steady state over a period of about 2 days. In some embodiments, the plasma concentration of the compound is maintained at steady state over a period of about 3 days. In some embodiments, the plasma concentration of the compound is maintained at steady state over a period of about 4 days. In some embodiments, the plasma concentration of the compound is maintained at steady state over a period of about 5 days. In some embodiments, the plasma concentration of the compound is maintained at steady state over a period of about 6 days. In some embodiments, the plasma concentration of the compound is maintained at steady state over a period of about 1 week. In some embodiments, the plasma concentration of the compound is maintained at steady state over a period of about 2 weeks. In some embodiments, the plasma concentration of the compound is maintained at steady state over a period of about 3 weeks. In some embodiments, the plasma concentration of the compound is maintained at steady state over a period of about 4 weeks. In some embodiments, the plasma concentration of the compound is maintained at steady state over a period of about 5 weeks. In some embodiments, the plasma concentration of the compound is maintained at steady state over a period of about 6 weeks. In some embodiments, the plasma concentration of the compound is maintained at steady state over a period of about 8 weeks. In some embodiments, the plasma concentration of the compound is maintained at steady state over a period of about 1 month. In some embodiments, the plasma concentration of the compound is maintained at steady state over a period of about 2 months. In some embodiments, the plasma concentration of the compound is maintained at steady state over a period of about 1 day to about 1 month. In some embodiments, the plasma concentration of the compound is maintained at steady state over a period of about 1 day to about 2 months. In some embodiments, the plasma concentration of the compound is maintained at steady state over a period of about 1 week to about 1 month. In some embodiments, the plasma concentration of the compound is maintained at steady state over a period of about 1 week to about 2 months. In some embodiments, the plasma concentration of the compound is maintained at steady state over a period of about 2 weeks to about 1 month. In some embodiments, the plasma concentration of the compound is maintained at steady state over a period of about 2 weeks to about 2 months. In some embodiments, the plasma concentration of the compound is maintained at steady state over a period of about 1 month to about 2 months.

In some embodiments, the term “long acting” refers to a release profile wherein the therapeutic effect of the compound is maintained for an extended period of time. In some embodiments, the therapeutic effect is maintained for a period of about 1 day. In some embodiments, the therapeutic effect is maintained for a period of about 2 days. In some embodiments, the therapeutic effect is maintained for a period of about 3 days. In some embodiments, the therapeutic effect is maintained for a period of about 4 days. In some embodiments, the therapeutic effect is maintained for a period of about 5 days. In some embodiments, the therapeutic effect is maintained for a period of about 6 days. In some embodiments, the therapeutic effect is maintained for a period of about 1 week. In some embodiments, the therapeutic effect is maintained for a period of about 2 weeks. In some embodiments, the therapeutic effect is maintained for a period of about 3 weeks. In some embodiments, the therapeutic effect is maintained for a period of about 4 weeks. In some embodiments, the therapeutic effect is maintained for a period of about 5 weeks. In some embodiments, the therapeutic effect is maintained for a period of about 6 weeks. In some embodiments, the therapeutic effect is maintained for a period of about 8 weeks. In some embodiments, the therapeutic effect is maintained for a period of about 1 month. In some embodiments, the therapeutic effect is maintained for a period of about 2 months. In some embodiments, the therapeutic effect is maintained for a period of about 1 week to about 1 month. In some embodiments, the therapeutic effect is maintained for a period of about 1 week to about 2 months. In some embodiments, the therapeutic effect is maintained for a period of about 2 weeks to about 1 month. In some embodiments, the therapeutic effect is maintained for a period of about 2 weeks to about 2 months. In some embodiments, the therapeutic effect is maintained for a period of about 1 month to about 2 months.

In some embodiments, a formulation of the present disclosure is administered in an amount sufficient to maintain a concentration of between about 1 ng/mL and about 10 ng/mL over a dosing cycle. In some embodiments, a formulation of the present disclosure is administered in an amount sufficient to maintain a concentration of between about 1 ng/mL and about 5 ng/mL over a dosing cycle. In some embodiments, a formulation of the present disclosure is administered in an amount sufficient to maintain a concentration of between about 1 ng/mL and about 3 ng/mL over a dosing cycle. In some embodiments, a formulation of the present disclosure is administered in an amount sufficient to maintain a concentration of at least 1 ng/mL of compound for at least 2 weeks after administration. In some embodiments, a formulation of the present disclosure is administered in an amount sufficient to maintain a concentration of at least 1 ng/mL of compound for at least 4 weeks after administration. In some embodiments, a formulation of the present disclosure is administered in an amount sufficient to maintain a concentration of at least 1 ng/mL of compound for at least 6 weeks after administration. In some embodiments, a formulation of the present disclosure is administered in an amount sufficient to maintain a concentration of at least 1 ng/mL of compound for at least 8 weeks after administration. In some embodiments, a formulation of the present disclosure is administered in an amount sufficient to maintain a concentration of at least 1 ng/mL of compound for at least 1 month after administration. In some embodiments, a formulation of the present disclosure is administered in an amount sufficient to maintain a concentration of at least 1 ng/mL of compound for at least 2 months after administration.

In some embodiments, a formulation of the present disclosure is administered in an amount sufficient to maintain a concentration of at least 2 ng/mL of compound for at least 2 weeks after administration. In some embodiments, a formulation of the present disclosure is administered in an amount sufficient to maintain a concentration of at least 2 ng/mL of compound for at least 4 weeks after administration. In some embodiments, a formulation of the present disclosure is administered in an amount sufficient to maintain a concentration of at least 2 ng/mL of compound for at least 6 weeks after administration. In some embodiments, a formulation of the present disclosure is administered in an amount sufficient to maintain a concentration of at least 2 ng/mL of compound for at least 8 weeks after administration. In some embodiments, a formulation of the present disclosure is administered in an amount sufficient to maintain a concentration of at least 2 ng/mL of compound for at least 1 month after administration. In some embodiments, a formulation of the present disclosure is administered in an amount sufficient to maintain a concentration of at least 2 ng/mL of compound for at least 2 months after administration.

In some embodiments, a formulation of the present disclosure is administered in an amount sufficient to maintain a concentration of at least 3 ng/mL of compound for at least 2 weeks after administration. In some embodiments, a formulation of the present disclosure is administered in an amount sufficient to maintain a concentration of at least 3 ng/mL of compound for at least 4 weeks after administration. In some embodiments, a formulation of the present disclosure is administered in an amount sufficient to maintain a concentration of at least 3 ng/mL of compound for at least 6 weeks after administration. In some embodiments, a formulation of the present disclosure is administered in an amount sufficient to maintain a concentration of at least 3 ng/mL of compound for at least 8 weeks after administration. In some embodiments, a formulation of the present disclosure is administered in an amount sufficient to maintain a concentration of at least 3 ng/mL of compound for at least 1 month after administration. In some embodiments, a formulation of the present disclosure is administered in an amount sufficient to maintain a concentration of at least 3 ng/mL of compound for at least 2 months after administration.

In some embodiments, a formulation of the present disclosure is administered in an amount sufficient to maintain a concentration of at least 5 ng/mL of compound for at least 2 weeks after administration. In some embodiments, a formulation of the present disclosure is administered in an amount sufficient to maintain a concentration of at least 5 ng/mL of compound for at least 4 weeks after administration. In some embodiments, a formulation of the present disclosure is administered in an amount sufficient to maintain a concentration of at least 5 ng/mL of compound for at least 6 weeks after administration. In some embodiments, a formulation of the present disclosure is administered in an amount sufficient to maintain a concentration of at least 5 ng/mL of compound for at least 8 weeks after administration. In some embodiments, a formulation of the present disclosure is administered in an amount sufficient to maintain a concentration of at least 5 ng/mL of compound for at least 1 month after administration. In some embodiments, a formulation of the present disclosure is administered in an amount sufficient to maintain a concentration of at least 5 ng/mL of compound for at least 2 months after administration.

In some embodiments, a formulation of the present disclosure is administered in an amount sufficient to maintain a concentration of at least 10 ng/mL of compound for at least 2 weeks after administration. In some embodiments, a formulation of the present disclosure is administered in an amount sufficient to maintain a concentration of at least 10 ng/mL of compound for at least 4 weeks after administration. In some embodiments, a formulation of the present disclosure is administered in an amount sufficient to maintain a concentration of at least 10 ng/mL of compound for at least 6 weeks after administration. In some embodiments, a formulation of the present disclosure is administered in an amount sufficient to maintain a concentration of at least 10 ng/mL of compound for at least 8 weeks after administration. In some embodiments, a formulation of the present disclosure is administered in an amount sufficient to maintain a concentration of at least 10 ng/mL of compound for at least 1 month after administration. In some embodiments, a formulation of the present disclosure is administered in an amount sufficient to maintain a concentration of at least 10 ng/mL of compound for at least 2 months after administration.

In one aspect, the present disclosure provides a pharmaceutically acceptable composition comprising a long-acting formulation of a compound that reduces the level and/or activity of a protein, or a pharmaceutically acceptable salt thereof and a pharmaceutically acceptable excipient.

The term “injectable” as used herein refers to an aseptic preparation acting in such a way that a commonly injected liquid-phase drug is directly introduced into the body via the skin, muscle, vein, etc. In some embodiments, the term “injectable” refers to subcutaneous and/or intramuscular preparations. In some embodiments, the term “injectable” refers to subcutaneous preparations. In some embodiments, the term “injectable” refers to intramuscular preparations.

The term “formulation” or “composition” or “dosage form” as used herein refers to physically discrete units, wherein each unit containing a predetermined quantity of active ingredient in association with a pharmaceutically acceptable excipient.

Long-acting formulations comprise a variety of sub-types, including, but not limited to, nanosuspensions, in situ gel and microsphere poly (lactic-co-glycolic) acid (PLGA), in situ sucrose acetate isobutyrate (SAIB) gels, and SAIB nanosuspensions. In some embodiments, a formulation of the present disclosure comprises a nanosuspension. In some embodiments, a formulation of the present disclosure comprises a microsuspension. In some embodiments, a formulation of the present disclosure comprises PLGA microspheres. In some embodiments, a formulation of the present disclosure comprises a PLGA in situ gel. In some embodiments, a formulation of the present disclosure comprises an in situ SAIB gel. In some embodiments, a formulation of the present disclosure comprises an in situ SAIB nanosuspension. In some embodiments, a formulation of the present disclosure is lyophilized. In some embodiments, a formulation of the present disclosure is frozen. In some embodiments, a formulation of the present disclosure is a solution. In some embodiments, a formulation of the present disclosure is a suspension. In some embodiments, a formulation of the present disclosure is aqueous. In some embodiments, a formulation of the present disclosure is oil-based. In some embodiments, a formulation of the present disclosure is crystalline. In some embodiments, a formulation of the present disclosure is amorphous.

### Nanosuspension/Microsuspension

A nanosuspension is generally a disperse system of solid-in-liquid or solid-in-semisolid, wherein the dispersed phase comprises one or more compounds. On average, the diameter of the dispersed phase is between about 10 nm and about 999 nm for nanosuspensions. Should the average particle size (e.g., D₅₀) of the drug particle reach 1 μm, the formulation may be referred to as a microsuspension. A nanosuspension and/or microsuspension can be surfactant-free but can also comprise surfactants and/or other excipients. A nanosuspension and/or microsuspension can be lyophilized or spray dried, and the nanoparticles of a nanosuspension and/or microparticles of a microsuspension can also be incorporated into a solid carrier matrix.

Advantages to a nanosuspension and/or microsuspension formulation include, but are not limited to, an ability to enhance solubility and bioavailability of a drug, higher drug loading, dose reduction, enhance physical and chemical drug stability, and provide passive drug targeting. Nanosuspension and/or microsuspension-based drug particle size reduction results in an increase in the effective surface area and therefore can increase in intrinsic solubility as well as enhance dissolution rate.

A nanosuspension and/or microsuspension of the present disclosure can be processed and/or manufactured by several different methods including, but not limited to media milling, jet milling, high pressure homogenization, microfluidizer, precipitation, emulsion, microemulsion, melt emulsification, supercritical fluid, nanoedge, and/or combinations thereof. In some embodiments, a nanosuspension and/or microsuspension of the present disclosure is processed and/or manufactured by media milling. In some embodiments, a nanosuspension and/or microsuspension of the present disclosure is processed and/or manufactured by jet milling. In some embodiments, a nanosuspension and/or microsuspension of the present disclosure is processed and/or manufactured by high pressure homogenization. In some embodiments, a nanosuspension and/or microsuspension of the present disclosure is processed and/or manufactured by a microfluidizer. In some embodiments, a nanosuspension and/or microsuspension of the present disclosure is processed and/or manufactured by precipitation. In some embodiments, a nanosuspension and/or microsuspension of the present disclosure is processed and/or manufactured by emulsion. In some embodiments, a nanosuspension and/or microsuspension of the present disclosure is processed and/or manufactured by microemulsion. In some embodiments, a nanosuspension and/or microsuspension of the present disclosure is processed and/or manufactured by melt emulsification. In some embodiments, a nanosuspension and/or microsuspension of the present disclosure is processed and/or manufactured by supercritical fluid. In some embodiments, a nanosuspension and/or microsuspension of the present disclosure is processed and/or manufactured by nanoedge.

Nanosuspension and/or microsuspension particle size and particle distribution (polydispersity index) can influence stability, saturation solubility of drug and dissolution velocity as well as in vivo performance. Photon Correlation Spectroscopy (PCS) and Laser Diffractometry (LD) analysis are commonly used techniques to measure such parameters. A low value of polydispersity index ranging between 0.1 and 0.3 signifies homogeneity and stability of a nanosuspension and/or microsuspension, while a higher value is indicative of broad and heterogeneous distribution in the dispersed phase. In some embodiments, a nanosuspension and/or microsuspension of the present invention has a low polydispersity index. In some embodiments, a nanosuspension and/or microsuspension of the present invention has a polydispersity index of between about 0.1 and about 0.3.

In one aspect, the present disclosure provides a pharmaceutically acceptable composition comprising a long-acting formulation of a compound that reduces the level and/or activity of a protein, or a pharmaceutically acceptable salt thereof, and a pharmaceutically acceptable excipient.

In some embodiments, a nanosuspension and/or microsuspension of the present disclosure comprises an excipient. In some embodiments, the excipient includes a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof. In some embodiments, the excipient is used to reconstitute a lyophilized, frozen, spray dried, or powdered nanosuspension and/or microsuspension prior to administration. In some embodiments, a nanosuspension and/or microsuspension of the present disclosure comprises TWEEN®20 (polysorbate 20). In some embodiments, a nanosuspension and/or microsuspension of the present disclosure comprises sodium carboxymethyl cellulose (CMC Na). In some embodiments, a nanosuspension and/or microsuspension of the present disclosure comprises poly(vinyl pyrrolidone) (PVP). In some embodiments, a nanosuspension and/or microsuspension of the present disclosure comprises poly(vinyl pyrrolidone) K12 (PVP K12). In some embodiments, a nanosuspension and/or microsuspension of the present disclosure comprises saline. In some embodiments, a nanosuspension and/or microsuspension of the present disclosure comprises TWEEN®20. In some embodiments, a nanosuspension and/or microsuspension of the present disclosure comprises PLGA. In some embodiments, the PLGA is in a ratio of 50% lactic acid to 50% glycolic acid. In some embodiments, the PLGA has an acidic N-terminus. In some embodiments, a nanosuspension and/or microsuspension of the present disclosure comprises saline and TWEEN®20. In some embodiments, a nanosuspension and/or microsuspension of the present disclosure comprises olive oil. In some embodiments, a nanosuspension and/or microsuspension of the present disclosure comprises soybean oil. In some embodiments, a nanosuspension and/or microsuspension of the present disclosure comprises sesame oil. In some embodiments, a nanosuspension and/or microsuspension of the present disclosure comprises water.

Particle size distribution (PSD) is represented as D₁₀, D₅₀, and D₉₀ values. D₁₀ indicates the size below which 10% of all particles in the formulation are found. D₅₀ indicates the size below which 50% of all particles in the formulation are found. D₉₀ indicates the size below which 90% of all particles in the formulation are found. In some embodiments, particle size is represented as D₁₀ of the formulation. In some embodiments, particle size is represented as D₅₀ of the formulation. In some embodiments, particle size is represented as D₉₀ of the formulation. In some embodiments, particle size is represented as particle size distribution (PSD). In some embodiments, the size of particles in a formulation is measured as an average particle size (e.g., D₅₀). In some embodiments, the size of particles of a nanosuspension of the present disclosure is measured as an average particle size.

In some embodiments, a nanosuspension comprises an average particle size (e.g., D₅₀) of between about 100 nm and about 1000 nm.

In some embodiments, a formulation of the present disclosure is a microsuspension. In some embodiments, the size of particles of a microsuspension of the present disclosure is measured as an average particle size (e.g., D₅₀). In some embodiments, a microsuspension comprises an average particle size (e.g., D₅₀) of between about 1 μm and about 50 μm.

In some embodiments, a nanosuspension and/or microsuspension of the present disclosure comprises between about 30 mg and about 200 mg of compound per g of formulation.

A nanosuspension and/or microsuspension can be described by identifying weight percentages of the total mass of the formulation of each component of the formulation. In some embodiments, a nanosuspension formulation and/or microsuspension formulation as described herein comprises a compound of the present disclosure, water and/or oil, and one or more further excipients.

In some embodiments, a nanosuspension and/or microsuspension formulation of the present disclosure comprises a weight percentage of a compound of the present disclosure of the total mass of the formulation. In some embodiments, a nanosuspension and/or microsuspension of the present disclosure comprises between about 1% compound by weight and about 20% of compound by weight of the total mass of the formulation.

In further embodiments, a nanosuspension and/or microsuspension formulation comprises a percentage by weight of one or more excipients of the total mass of the formulation. In some embodiments, a nanosuspension and/or microsuspension of the present disclosure comprises between about 0.1% excipient by weight and about 5% excipient by weight of the total mass of the formulation.

In further embodiments, the remainder of the nanosuspension and/or microsuspension comprises water and/or oil, sufficient to make the total weight percent of the formulation equal to 100%. In some embodiments, the percentage by weight of water and/or oil of the total mass of the formulation is 100% minus the sum of the percentage of compound by weight and the percentage of one of more excipients by weight of the total mass of the formulation. In some embodiments, a nanosuspension and/or microsuspension comprises between about 75% water and/or oil by weight and about 97% water and/or oil by weight of the total mass of the formulation.

A liquid nanosuspension and/or microsuspension can have different measurable properties that alter drug availability, one of which is viscosity. In some embodiments, a nanosuspension and/or microsuspension of the present disclosure has a viscosity of between about 1 and about 25 cp.

A nanosuspension and/or microsuspension of the present disclosure may be lyophilized, frozen, spray dried, powdered, aqueous, or oil-based. In some embodiments, a nanosuspension and/or microsuspension of the present disclosure is lyophilized. In some embodiments, a nanosuspension and/or microsuspension of the present disclosure comprises a cryoprotectant. In some embodiments, the cryoprotectant is trehalose, sucrose, mannitol, PVP, PVA, PEG, amino acids such as glycine or a combination thereof. In some embodiments, a nanosuspension and/or microsuspension of the present disclosure is frozen. In some embodiments, a nanosuspension and/or microsuspension of the present disclosure is spray dried. In some embodiments, a nanosuspension and/or microsuspension of the present disclosure is powdered. In some embodiments, a nanosuspension and/or microsuspension formulation of the present disclosure is crystalline. In some embodiments, a nanosuspension and/or microsuspension formulation of the present disclosure is partially crystalline (mixture of crystalline and amorphous) In some embodiments, a nanosuspension and/or microsuspension formulation of the present disclosure is amorphous (e.g., the amorphous microsuspension is media milled in oil).

### Poly (Lactic-Co-Glycolic) Acid (PLGA)-Based Microspheres and In Situ Gels

Poly (lactic-so-glycolic) acid (PLGA) formulations have been used for over 30 years to deliver small molecules, peptides, and proteins for periods ranging from 1 week to 6 months. Around 20 PLGA formulated drugs have been approved by the FDA. Most commonly, PLGA formulations are microspheres, but they can also be in the form of in situ gels and solid implants. As used herein, the terms microparticle and microsphere are interchangeable. PLGA is typically defined by its molecular weight, its lactic acid to glycolic acid (L:G) ratio, by the end group attached, and also by the molecular shape (e.g., linear or branched).

In some embodiments, a formulation of the present disclosure comprises PLGA microspheres. In some embodiments, a formulation of the present disclosure comprises in situ PLGA gels. In some embodiments, a formulation of the present disclosure comprises solid PLGA implants. In some embodiments, the PLGA is linear. In some embodiments, the PLGA is branched. In some embodiments, the PLGA has an ester N-terminus (e.g., RG502). In some embodiments, the PLGA has an acid N-terminus (e.g., RG502H).

In one aspect, the present disclosure provides a pharmaceutically acceptable composition comprising a long-acting formulation of a compound that reduces the level and/or activity of BRD9, or a pharmaceutically acceptable salt thereof, and a pharmaceutically acceptable excipient.

In some embodiments, a PLGA microsphere and/or PLGA gel formulation of the present disclosure comprises one or more excipients. In some embodiments, a PLGA microsphere and/or PLGA gel formulation of the present disclosure comprises a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof. In some embodiments, the excipient is used to reconstitute a lyophilized, frozen, spray dried, or powdered PLGA microsphere formulations prior to administration. In some embodiments, a PLGA microsphere and/or PLGA gel formulation of the present disclosure is suspended and dispersed in an excipient. In some embodiments, a PLGA microsphere and/or PLGA gel formulation of the present disclosure comprises a solvent, diluent, surfactant, or a combination thereof. In some embodiments, a PLGA microsphere and/or PLGA gel formulation of the present disclosure is suspended and dispersed in poly(vinyl alcohol) (PVA). In some embodiments, a PLGA microsphere and/or PLGA gel formulation of the present disclosure is suspended and dispersed in POLOXAMER®188 (P-188). In some embodiments, a PLGA microsphere and/or PLGA gel formulation of the present disclosure is suspended and dispersed in POLOXAMER®407 (P-407).

In situ forming PLGA gel implants/depots are formed by mixing drug powder with PLGA dissolved in an excipient (e.g., a solvent) just before injection. After injection, the excipient, which is generally a water-miscible solvent, is diluted with water at the injection site, leading to gel formation and solidification of the drug/PLGA mixture. In some embodiments, a PLGA in situ gel formulation of the present disclosure comprises one or more excipients. In some embodiments, the excipient comprises a solvent. In some embodiments, the solvent is dichloromethane (DCM), dimethylacetamide (DMA), dimethyl sulfoxide (DMSO), dimethyl formamide (DMF), ethyl acetate, N-methyl-2-pyrrolidone (NMP), methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, or combinations thereof. In some embodiments, the solvent is dimethyl sulfoxide (DMSO). In some embodiments, the solvent is dimethyl formamide (DMF). In some embodiments the solvent is ethyl acetate, N-methyl-2-pyrrolidone (NMP).

Microparticles or microspheres are a common form of PLGA formulation. Particle size distribution (PSD) is represented as D₁₀, D₅₀, and D₉₀ values. D₁₀ indicates the size below which 10% of all particles in the formulation are found. D₅₀ indicates the size below which 50% of all particles in the formulation are found. Do indicates the size below which 90% of all particles in the formulation are found. In some embodiments, particle size is represented as D₁₀ of the formulation. In some embodiments, particle size is represented as D₅₀ of the formulation. In some embodiments, particle size is represented as Do of the formulation. In some embodiments, particle size is represented as particle size distribution (PSD). In some embodiments, the size of particles in a formulation is measured as an average particle size (e.g., D₅₀). In some embodiments, the size of a PLGA microsphere is measured as an average particle size.

In some embodiments, the average size of a PLGA microsphere is about 100 μm. In some embodiments, the average size of a PLGA microsphere is between about 90 μm and about 100 μm. In some embodiments, the average size of a PLGA microsphere is between about 80 μm and about 90 μm. In some embodiments, the average size of a PLGA microsphere is between about 70 μm and about 80 μm. In some embodiments, the average size of a PLGA microsphere is between about 60 μm and about 70 μm. In some embodiments, the average size of a PLGA microsphere is between about 50 μm and about 60 μm. In some embodiments, the average size of a PLGA microsphere is between about 40 μm and about 50 μm. In some embodiments, the average size of a PLGA microsphere is between about 30 μm and about 40 μm. In some embodiments, the average size of a PLGA microsphere is between about 20 μm and about 30 μm. In some embodiments, the average size of a PLGA microsphere is between about 10 μm and about 20 μm. In some embodiments, the average size of a PLGA microsphere is between about 1 μm and about 10 μm. In some embodiments, the average size of a PLGA microsphere is between about 5 μm and about 10 μm. In some embodiments, the average size of a PLGA microsphere is between about 5 μm and about 100 μm. In some embodiments, the average size of a PLGA microsphere is between about 1 μm and about 100 μm.

In some embodiments, a PLGA microsphere and/or in situ PLGA gel formulation of the present disclosure comprises between about 50 mg and about 800 mg (e.g., 50-100 mg, 50-200 mg, 80-200 mg, 50-300 mg, 50-400 mg, 50-500 mg, 50-600 mg, 50-700 mg, 100-200 mg, 100-400 mg, 100-500 mg, 100-700 mg, 100-800 mg, 200-400 mg, 200-500 mg, 200-800 mg, 500-800 mg) of compound per g of formulation. In some embodiments, a PLGA microsphere and/or PLGA in situ gel formulation of the present disclosure comprises between about 50 mg and about 100 mg of compound per g of formulation. In some embodiments, a PLGA microsphere and/or PLGA in situ gel formulation of the present disclosure comprises between about 50 mg and about 200 mg of compound per g of formulation. In some embodiments, a PLGA microsphere and/or PLGA in situ gel formulation of the present disclosure comprises between about 80 mg and about 200 mg of compound per g of formulation. In some embodiments, a PLGA microsphere and/or PLGA in situ gel formulation of the present disclosure comprises between about 50 mg and about 300 mg of compound per g of formulation. In some embodiments, a PLGA microsphere and/or PLGA in situ gel formulation of the present disclosure comprises between about 50 mg and about 400 mg of compound per g of formulation. In some embodiments, a PLGA microsphere and/or PLGA in situ gel formulation of the present disclosure comprises between about 50 mg and about 500 mg of compound per g of formulation. In some embodiments, a PLGA microsphere and/or PLGA in situ gel formulation of the present disclosure comprises between about 50 mg and about 600 mg of compound per g of formulation. In some embodiments, a PLGA microsphere and/or PLGA in situ gel formulation of the present disclosure comprises between about 50 mg and about 700 mg of compound per g of formulation. In some embodiments, a PLGA microsphere and/or PLGA in situ gel formulation of the present disclosure comprises between about 100 mg and about 200 mg of compound per g of formulation. In some embodiments, a PLGA microsphere and/or PLGA in situ gel formulation of the present disclosure comprises between about 100 mg and about 400 mg of compound per g of formulation. In some embodiments, a PLGA microsphere and/or PLGA in situ gel formulation of the present disclosure comprises between about 100 mg and about 500 mg of compound per g of formulation. In some embodiments, a PLGA microsphere and/or PLGA in situ gel formulation of the present disclosure comprises between about 100 mg and about 700 mg of compound per g of formulation. In some embodiments, a PLGA microsphere and/or PLGA in situ gel formulation of the present disclosure comprises between about 100 mg and about 800 mg of compound per g of formulation. In some embodiments, a PLGA microsphere and/or PLGA in situ gel formulation of the present disclosure comprises between about 200 mg and about 400 mg of compound per g of formulation. In some embodiments, a PLGA microsphere and/or PLGA in situ gel formulation of the present disclosure comprises between about 200 mg and about 500 mg of compound per g of formulation. In some embodiments, a PLGA microsphere and/or PLGA in situ gel formulation of the present disclosure comprises between about 200 mg and about 800 mg of compound per g of formulation. In some embodiments, a PLGA microsphere and/or PLGA in situ gel formulation of the present disclosure comprises between about 500 mg and about 800 mg of compound per g of formulation.

In some embodiments, a PLGA microsphere formulation and/or in situ PLGA gel formulation of the present disclosure comprises between about 80 mg and about 900 mg (e.g., 80-200 mg, 100-200 mg, 100-400 mg, 100-500 mg, 100-700 mg, 200-400 mg, 200-500 mg, 200-900 mg, 500-900 mg) of PLGA per g of formulation. In some embodiments, a PLGA microsphere and/or PLGA in situ gel formulation of the present disclosure comprises between about 80 mg and about 200 mg of PLGA per g of formulation. In some embodiments, a PLGA microsphere and/or PLGA in situ gel formulation of the present disclosure comprises between about 100 mg and about 200 mg of PLGA per g of formulation. In some embodiments, a PLGA microsphere and/or PLGA in situ gel formulation of the present disclosure comprises between about 100 mg and about 400 mg of PLGA per g of formulation. In some embodiments, a PLGA microsphere and/or PLGA in situ gel formulation of the present disclosure comprises between about 100 mg and about 500 mg of PLGA per g of formulation. In some embodiments, a PLGA microsphere and/or PLGA in situ gel formulation of the present disclosure comprises between about 100 mg and about 700 mg of PLGA per g of formulation. In some embodiments, a PLGA microsphere and/or PLGA in situ gel formulation of the present disclosure comprises between about 200 mg and about 400 mg of PLGA per g of formulation. In some embodiments, a PLGA microsphere and/or PLGA in situ gel formulation of the present disclosure comprises between about 200 mg and about 500 mg of PLGA per g of formulation. In some embodiments, a PLGA microsphere and/or PLGA in situ gel formulation of the present disclosure comprises between about 200 mg and about 900 mg of PLGA per g of formulation. In some embodiments, a PLGA microsphere and/or PLGA in situ gel formulation of the present disclosure comprises between about 500 mg and about 900 mg of PLGA per g of formulation.

A PLGA microsphere formulation and/or PLGA gel formulation can be described by identifying weight percentages of the total mass of the formulation of each component of the formulation. In some embodiments, a PLGA microsphere and/or in situ PLGA gel formulation as described herein comprises a compound of the present disclosure, PLGA, and one or more excipients. In some embodiments, one or more excipients is evaporated from a PLGA microsphere formulation during formulation processing. The weight percentages for all components of a formulation should add up to 100%.

In some embodiments, a PLGA microsphere and/or PLGA in situ gel formulation can comprise a percentage of a compound of the present disclosure, of the total mass of the formulation. In some embodiments, a PLGA microsphere and/or PLGA in situ gel formulation of the present disclosure comprises between about 5% compound by weight and about 70% compound by weight of the total mass of the formulation. In some embodiments, a PLGA microsphere formulation of the present disclosure comprises between about 10% compound by weight and about 70% compound by weight of the total mass of the formulation. In some embodiments, a PLGA microsphere formulation of the present disclosure comprises between about 20% compound by weight and about 30% compound by weight of the total mass of the formulation. In some embodiments, a PLGA in situ gel formulation of the present disclosure comprises between about 5% compound by weight and about 20% compound by weight of the total mass of the formulation. In some embodiments, a PLGA in situ gel formulation of the present disclosure comprises between about 8% compound by weight and about 20% compound by weight of the total mass of the formulation. In some embodiments, a PLGA in situ gel formulation of the present disclosure comprises between about 8% compound by weight and about 10% compound by weight of the total mass of the formulation. In some embodiments, a PLGA in situ gel formulation of the present disclosure comprises between about 10% compound by weight and about 20% compound by weight of the total mass of the formulation.

In further embodiments, a PLGA microsphere and/or in situ PLGA gel formulation comprises a weight percentage of PLGA of the total mass of the formulation. In some embodiments, a PLGA microsphere and/or in situ PLGA gel formulation of the present disclosure comprises between about 10% by weight of PLGA and about 90% by weight of PLGA of the total mass of the formulation. In some embodiments, a PLGA microsphere and/or in situ PLGA gel formulation of the present disclosure comprises between about 20% by weight of PLGA and about 90% by weight of PLGA of the total mass of the formulation. In some embodiments, a PLGA microsphere and/or in situ PLGA gel formulation of the present disclosure comprises between about 30% by weight of PLGA and about 90% by weight of PLGA of the total mass of the formulation. In some embodiments, a PLGA microsphere and/or in situ PLGA gel formulation of the present disclosure comprises between about 30% by weight of PLGA and about 80% by weight of PLGA of the total mass of the formulation. In some embodiments, a PLGA microsphere and/or in situ PLGA gel formulation of the present disclosure comprises between about 30% by weight of PLGA and about 70% by weight of PLGA of the total mass of the formulation. In some embodiments, a PLGA microsphere and/or in situ PLGA gel formulation of the present disclosure comprises between about 30% by weight of PLGA and about 60% by weight of PLGA of the total mass of the formulation. In some embodiments, a PLGA microsphere and/or in situ PLGA gel formulation of the present disclosure comprises between about 30% by weight of PLGA and about 50% by weight of PLGA of the total mass of the formulation. In some embodiments, a PLGA microsphere and/or in situ PLGA gel formulation of the present disclosure comprises between about 30% by weight of PLGA and about 40% by weight of PLGA of the total mass of the formulation. In some embodiments, a PLGA microsphere and/or in situ PLGA gel formulation of the present disclosure comprises between about 10% by weight of PLGA and about 40% by weight of PLGA of the total mass of the formulation. In some embodiments, a PLGA microsphere and/or in situ PLGA gel formulation of the present disclosure comprises between about 10% by weight of PLGA and about 30% by weight of PLGA of the total mass of the formulation. In some embodiments, a PLGA microsphere and/or in situ PLGA gel formulation of the present disclosure comprises between about 20% by weight of PLGA and about 30% by weight of PLGA of the total mass of the formulation. In some embodiments, a PLGA microsphere and/or in situ PLGA gel formulation of the present disclosure comprises between about 10% by weight of PLGA and about 20% by weight of PLGA of the total mass of the formulation.

In further embodiments, a PLGA microsphere and/or in situ PLGA gel formulation can comprise a percentage of one or more excipients (e.g., polymer), of the total mass of the formulation. In some embodiments, a PLGA microsphere and/or in situ PLGA gel formulation of the present disclosure comprises between about 50% excipient by weight and about 75% excipient by weight of the total mass of the formulation. In some embodiments, a PLGA microsphere and/or in situ PLGA gel formulation of the present disclosure comprises between about 50% excipient by weight and about 70% excipient by weight of the total mass of the formulation. In some embodiments, a PLGA microsphere and/or in situ PLGA gel formulation of the present disclosure comprises between about 60% excipient by weight and about 70% excipient by weight of the total mass of the formulation. In some embodiments, a PLGA microsphere and/or in situ PLGA gel formulation of the present disclosure comprises between about 50% excipient by weight and about 60% excipient by weight of the total mass of the formulation.

In further embodiments, an excipient may be evaporated from a PLGA microsphere formulation as part of formulation processing. In some embodiments, a PLGA microsphere formulation of the present disclosure comprises between about 50% excipient by weight and about 75% excipient by weight of the total mass of the formulation. In some embodiments, a PLGA microsphere formulation of the present disclosure comprises between about 50% excipient by weight and about 70% excipient by weight of the total mass of the formulation that is evaporated during formulation processing. In some embodiments, a PLGA microsphere formulation of the present disclosure comprises between about 60% excipient by weight and about 70% excipient by weight of the total mass of the formulation that is evaporated during formulation processing. In some embodiments, a PLGA microsphere formulation of the present disclosure comprises between about 50% excipient by weight and about 60% excipient by weight of the total mass of the formulation that is evaporated during formulation processing.

PLGA properties such as molecular weight and/or L:G ratios can affect final structures of microspheres, as they influence other events during microparticle formation, such as PLGA coalescence, shell formation, and solidification. This, in turn, impacts formation of pores throughout the microspheres, ultimately determining the initial burst and subsequent drug release kinetics. PLGAs with different L:G ratios dissolve in different solvents, making the L:G ratio even more important. As the L:G ratio increases, i.e., as the lactide portion increases, PLGA dissolves in a larger number of solvents. When the L:G ratio is smaller than 50:50 (i.e., more glycolide), the PLGA dissolves generally in only in highly fluorinated solvents, such as hexafluoroisopropanol. In some embodiments, a PLGA microsphere formulation of the present disclosure comprises a PLGA ratio of lactic acid to glycolic acid. In some embodiments, an in situ PLGA gel formulation of the present disclosure comprises a PLGA ratio of lactic acid to glycolic acid. In some embodiments, the PLGA ratio of lactic acid to glycolic acid is 95% lactic acid to 5% glycolic acid. In some embodiments, the PLGA ratio of lactic acid to glycolic acid is 90% lactic acid to 10% glycolic acid. In some embodiments, the PLGA ratio of lactic acid to glycolic acid is 85% lactic acid to 15% glycolic acid. In some embodiments, the PLGA ratio of lactic acid to glycolic acid is 80% lactic acid to 20% glycolic acid. In some embodiments, the PLGA ratio of lactic acid to glycolic acid is 75% lactic acid to 25% glycolic acid. In some embodiments, the PLGA ratio of lactic acid to glycolic acid is 70% lactic acid to 30% glycolic acid. In some embodiments, the PLGA ratio of lactic acid to glycolic acid is 65% lactic acid to 35% glycolic acid. In some embodiments, the PLGA ratio of lactic acid to glycolic acid is 60% lactic acid to 40% glycolic acid. In some embodiments, the PLGA ratio of lactic acid to glycolic acid is 55% lactic acid to 45% glycolic acid. In some embodiments, the PLGA ratio of lactic acid to glycolic acid is 50% lactic acid to 50% glycolic acid. In some embodiments, the PLGA ratio of lactic acid to glycolic acid is 45% lactic acid to 55% glycolic acid. In some embodiments, the PLGA ratio of lactic acid to glycolic acid is 40% lactic acid to 60% glycolic acid. In some embodiments, the PLGA ratio of lactic acid to glycolic acid is 35% lactic acid to 65% glycolic acid. In some embodiments, the PLGA ratio of lactic acid to glycolic acid is 30% lactic acid to 70% glycolic acid. In some embodiments, the PLGA ratio of lactic acid to glycolic acid is 25% lactic acid to 75% glycolic acid. In some embodiments, the PLGA ratio of lactic acid to glycolic acid is 20% lactic acid to 80% glycolic acid. In some embodiments, the PLGA ratio of lactic acid to glycolic acid is 15% lactic acid to 85% glycolic acid. In some embodiments, the PLGA ratio of lactic acid to glycolic acid is 10% lactic acid to 90% glycolic acid. In some embodiments, the PLGA ratio of lactic acid to glycolic acid is 5% lactic acid to 95% glycolic acid. In some embodiments, the formulation comprises a mixture of one or more PLGA ratios of lactic acid to glycolic acid. In some embodiments, the PLGA has an ester N-terminus (e.g., RG502). In some embodiments, the PLGA has an acid N-terminus (e.g., RG502H).

Excipient-dependent PLGA solubility affects molecular dimensions and ultimately the solution viscosity. The solution viscosity in turn significantly impacts the droplet breakage and resultant emulsion microdroplet size distribution. In some embodiments, a PLGA microsphere and/or in situ PLGA gel formulation of the present disclosure has a viscosity of between about 1 and about 25 cp. In some embodiments, a PLGA microsphere and/or in situ PLGA gel formulation of the present disclosure has a viscosity of between about 1 and about 5 cp. In some embodiments, a PLGA microsphere and/or in situ PLGA gel formulation of the present disclosure has a viscosity of between about 1 and about 10 cp. In some embodiments, a PLGA microsphere and/or in situ PLGA gel formulation of the present disclosure has a viscosity of between about 1 and about 15 cp. In some embodiments, a PLGA microsphere and/or in situ PLGA gel formulation of the present disclosure has a viscosity of between about 1 and about 20 cp.

In some embodiments, a PLGA microsphere formulation and/or in situ PLGA gel formulation of the present disclosure is lyophilized. In some embodiments, a PLGA microsphere formulation and/or in situ PLGA gel formulation of the present disclosure is frozen. In some embodiments, a PLGA microsphere formulation and/or in situ PLGA gel formulation of the present disclosure is spray dried. In some embodiments, a PLGA microsphere formulation and/or in situ PLGA gel formulation of the present disclosure is powdered. In some embodiments, a PLGA microsphere formulation and/or in situ PLGA gel formulation of the present disclosure is aqueous. In some embodiments, a PLGA microsphere formulation and/or in situ PLGA gel formulation of the present disclosure is oil-based. In some embodiments, a PLGA microsphere and/or in situ PLGA gel formulation of the present disclosure is crystalline. In some embodiments, a PLGA microsphere and/or in situ PLGA gel formulation of the present disclosure is amorphous.

### Sucrose Acetate Isobutyrate (SAIB)-Based Nanosuspensions and In Situ Gels

In one aspect, the present disclosure provides a pharmaceutically acceptable composition comprising a long-acting formulation of a compound that reduces the level and/or activity of a protein, or a pharmaceutically acceptable salt thereof, and a pharmaceutically acceptable excipient.

Similar to PLGA gels, in situ forming sucrose acetate isobutyrate (SAIB) gel implants are typically formed by mixing drug powder with SAIB dissolved in an excipient (e.g., a solvent), just before injection. After injection, the solvent, which is generally a water-miscible solvent, is diluted with water at the injection site, leading to gel formation and solidification of the drug/SAIB mixture. SAIB can also form nanosuspensions.

In some embodiments, a formulation of the present disclosure comprises a sucrose acetate isobutyrate (SAIB) nanosuspension and/or SAIB in situ gel. In some embodiments, the formulation comprises a pharmaceutically acceptable excipient. In some embodiments, the excipient includes a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof. In some embodiments, the excipient is a solvent. In some embodiments, the solvent is dichloromethane, dimethyl sulfoxide, dimethyl formamide, ethyl acetate, methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, or combinations thereof. In some embodiments, the solvent is dimethyl sulfoxide. In some embodiments, the surfactant is polysorbate 20 [TWEEN®20], polysorbate 80 [TWEEN®80], POLOXAMER®188 (P-188), and/or POLOXAMER®407 (P-407).

In some embodiments, the excipient is used to reconstitute a lyophilized, frozen, spray dried, or powdered SAIB nanosuspension formulations prior to administration. In some embodiments, an SAIB nanosuspension formulation of the present disclosure is suspended and dispersed in an excipient. In some embodiments, an SAIB nanosuspension formulation of the present disclosure comprises a solvent, diluent, surfactant, or a combination thereof. In some embodiments, an SAIB nanosuspension formulation of the present disclosure is suspended and dispersed in dimethyl sulfoxide (DMSO).

Particle size distribution (PSD) is represented as D₁₀, D₅₀, and D₉₀ values. D₁₀ indicates the size below which 10% of all particles in the formulation are found. D₅₀ indicates the size below which 50% of all particles in the formulation are found. Do indicates the size below which 90% of all particles in the formulation are found. In some embodiments, particle size is represented as D₁₀ of the formulation. In some embodiments, particle size is represented as D₅₀ of the formulation. In some embodiments, particle size is represented as Do of the formulation. In some embodiments, particle size is represented as particle size distribution (PSD). In some embodiments, the size of particles in a formulation is measured as an average particle size (e.g., D₅₀). In some embodiments, the size of a particle in an SAIB nanosuspension is measured as an average particle size (e.g., D₅₀).

In some embodiments, an SAIB nanosuspension comprises an average particle size (e.g., D₅₀) of between about 100 nm and about 1000 nm.

In some embodiments, an SAIB nanosuspension and/or SAIB in situ gel formulation of the present disclosure comprises between about 50 mg and about 200 mg of compound per g of formulation.

In some embodiments, an SAIB nanosuspension formulation and/or in situ SAIB gel formulation of the present disclosure comprises between about 100 mg and about 800 mg of SAIB per g of formulation.

An SAIB nanosuspension formulation and/or an SAIB in situ gel formulation can be described by identifying weight percentages of the total mass of the formulation of each component of the formulation. In some embodiments, an SAIB nanosuspension formulation and/or an SAIB in situ gel formulation as described herein comprises a compound of the present disclosure, SAIB, and one or more excipients. The weight percentages for all components of a formulation should add up to 100%.

In some embodiments, an SAIB nanosuspension and/or SAIB in situ gel formulation of the present disclosure comprises a weight percentage of a compound of the present disclosure of the total mass of the formulation. In some embodiments, an SAIB nanosuspension and/or SAIB in situ gel formulation of the present disclosure comprises between about 5% compound by weight and about 20% of compound by weight of the total mass of the formulation.

In further embodiments, a SAIB microsphere and/or in situ SAIB gel formulation comprises a weight percentage of SAIB of the total mass of the formulation. In some embodiments, an SAIB nanosuspension and/or SAIB in situ gel formulation of the present disclosure comprises between about 10% by weight of SAIB and about 80% by weight of SAIB of the total mass of the formulation.

In further embodiments, an SAIB nanosuspension formulation and/or an SAIB in situ gel formulation comprises a percentage by weight of one or more excipients of the total mass of the formulation. In some embodiments, an SAIB nanosuspension and/or SAIB in situ gel formulation of the present disclosure comprises between about 0.1% excipient by weight and about 70% excipient by weight of the total mass of the formulation.

In some embodiments, an SAIB nanosuspension and/or SAIB in situ gel formulation of the present disclosure has a viscosity of between about 1 and about 25 cp

In some embodiments, an SAIB nanosuspension formulation and/or in situ SAIB gel formulation of the present disclosure is lyophilized. In some embodiments, an SAIB nanosuspension formulation and/or in situ SAIB gel formulation of the present disclosure is frozen. In some embodiments, an SAIB nanosuspension formulation and/or in situ SAIB gel formulation of the present disclosure is aqueous. In some embodiments, an SAIB nanosuspension formulation and/or in situ SAIB gel formulation of the present disclosure is oil-based. In some embodiments, an SAIB nanosuspension formulation and/or in situ SAIB gel formulation of the present disclosure is crystalline. In some embodiments, an SAIB nanosuspension and/or in situ SAIB gel formulation of the present disclosure is amorphous.

### Mechanical

Mechanical methods of preparing formulations include, but are not limited to, jet mills, disc mills, attritors and bead mills, ball mills, planetary mills, vibration and vibrocentrifugal mills, roller and centrifugal roller mills, extruders, hammer mills, knife mills, pin mills, and disintegrators.

In a jet mill, particles are accelerated to a desired speed in a gas flow, followed by a collision with an immobile body. In cyclone mills, particles collide with each other as they cross each other's trajectories rather than colliding with an immobile target. In some embodiments, a formulation of the present disclosure is prepared using a jet mill.

In disc mills, particles pass between two rotating discs, causing friction of the particles against the discs as well as grinding. Mills with a variable gap between the discs and discs with notches are used to increase milling efficiency. In some embodiments, a formulation of the present disclosure is prepared using a disc mill.

Attritors comprise a reactor with an impeller submerged in grinding bodies. As the impeller is rotating, it grinds the particles between the grinding bodies. In some embodiments, a formulation of the present disclosure is prepared using an attritor.

In ball mills, a large number of grinding bodies and the material being ground are placed inside a cylinder rotating around its central axis. Forces of friction between the walls of the cylinder and the grinding bodies cause the grinding bodies to move and grind the material. Sometimes grinding bodies are not used, and particles of the material grind themselves in a process known as “auto-grinding”. Planetary activator mills are a specific type of ball mill in which reservoirs are secured to a frame and rotate around a common axis, while the reactors rotate around their axes in the opposite direction. Disintegration occurs due to interactions between the material and grinding bodies, which are put into motion by centrifugal force, Coriolis force, and gravity. In some embodiments, a formulation of the present disclosure is prepared using a ball mill. In some embodiments, a formulation of the present disclosure is prepared using a planetary activator.

Vibration mills are used for fine and ultrafine grinding and use a vertical vibration for grinding instead of a circular motion used in ball mills. In some embodiments, a formulation of the present disclosure is prepared using a vibration mill.

Roller mills are a type of grinder in which the material is simultaneously subjected to two types of impact: abrasion and crushing. Centrifugal roller mills comprise roller axes that are oriented vertically, and the particles are ground as they travel between the rollers and the stator. In some embodiments, a formulation of the present disclosure is prepared using a roller mill.

Mechanical treatment of a material by an extruder is not necessarily accompanied by grinding. In an extruder, a screw (usually one or two) is the main working part, and as the screw rotates, it presses the particles along the treatment zones. In some embodiments, a formulation of the present disclosure is prepared using an extruder.

Hammer mills, knife mills, pin mills, and disintegrators all share a common feature in that the milling body is one or more discs with grinding elements secured on a surface. Material typically passes from the disc center into the treatment zone and is dispersed to the chamber periphery by centrifugal force, where it is exposed to grinding elements. In some embodiments, a formulation of the present disclosure is prepared using a hammer mill. In some embodiments, a formulation of the present disclosure is prepared using a knife mill. In some embodiments, a formulation of the present disclosure is prepared using a pin mill. In some embodiments, a formulation of the present disclosure is prepared using a disintegrator.

In some embodiments, a formulation of the present disclosure is prepared using a jet mill, disc mill, attritor, bead mill, ball mill, planetary mill, vibration mill, roller mill, centrifugal roller mills, extruder, hammer mill, knife mill, pin mill, or disintegrator.

### Excipients

Pharmaceutical formulations of the compounds described herein may additionally comprise a pharmaceutically acceptable excipient, which, as used herein, includes, but is not limited to, any and all solvents, dispersion media, diluents, or other liquid vehicles, dispersion or suspension aids, surfactants (surface active agents), isotonic agents, thickening or emulsifying agents, stabilizers, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Pharmaceutically acceptable excipients used in the manufacture of pharmaceutical compositions include, but are not limited to, inert diluents, surfactants and/or emulsifiers, solvents, preservatives, buffering agents, lubricating agents, and/or oils. Such excipients may optionally be included in the pharmaceutical formulations of the compounds described herein.

In some embodiments, a pharmaceutically acceptable excipient may be at least 95%, at least 96%, at least 97%, at least 98%, at least 99%, or 100% pure. In some embodiments, an excipient may be approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Exemplary solvents include, but are not limited to water, dichloromethane, dimethyl sulfoxide (DMSO), dimethyl formamide, ethyl acetate, ethyl formate, ethyl ether, methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, ethanol, isopropanol, butanol, propylene carbonate, methyl acetate, triacetin, benzyl alcohol, methylene chloride, chloroform, and/or a combination thereof.

Exemplary diluents include, but are not limited to, water, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, saline, dry starch, cornstarch, powdered sugar, and/or a combination thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, sodium carboxymethyl cellulose (CMC Na), cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or a combination thereof.

Exemplary surfactants (surface active agents) and/or emulsifiers and/or stabilizers include, but are not limited to, natural emulsifiers {e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. sodium carboxymethyl cellulose (CMC Na), powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate or polysorbate 20 [TWEEN®20], polyoxyethylene sorbitan or polysorbate 60 [TWEEN®60], polyoxyethylene sorbitan monooleate or polysorbate 80 [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®, PEG 4000), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, sodium dodecyl sulfate (SDS), PLUORINC®F 68, POLOXAMER®188, POLOXAMER®407, cetrimonium bromide, cetylpyridinium chloride, benzalkonium chloride, docusate sodium, povidone, transcutol, glycofurol, ethanol, isopropanol, bile salts, dipotassium glycyrrhizinate, and/or a combination thereof.

Exemplary polymers include, but are not limited to, poly(urethane), poly(siloxane), poly(silicone), poly(methyl methacrylate), poly(vinyl alcohol), poly(ethylene), poly(vinyl pyrrolidone) (PVP), poly(vinyl pyrrolidone) K12 (e.g., PVP K12, polyvidone, or povidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactide (PLA), polyglycolide (PGA), poly(lactide-co-glycolide) (PLGA), polyanhydrides, polyorthoester, and/or a combination thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol, and/or a combination thereof.

Exemplary preservatives include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERM ALL® 115, GERMABEN®II, NEOLONE™, KATHON™, and/or EUXYL®, and/or a combination thereof.

Exemplary cryoprotectants include, but are not limited to mannitol, glycerol, propylene glycol, dimethyl sulfoxide (DMSO), ethylene glycol, 2-methyl-2,4-pentadiol (MPD) or hexylene glycol, sucrose, trehalose, and/or a combination thereof.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or a combination thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and/or a combination thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or a combination thereof.

Excipients such as cocoa butter and suppository waxes, coloring agents, coating agents, sweetening, flavoring, and/or perfuming agents can be present in the composition.

In one aspect, the present disclosure provides a pharmaceutically acceptable composition comprising a long-acting formulation of a compound that reduces the level and/or activity of BRD9, or a pharmaceutically acceptable salt thereof.

In another aspect, the present disclosure provides a pharmaceutically acceptable composition comprising a long-acting formulation of the compound 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione or a pharmaceutically acceptable salt thereof.

In some embodiments, the formulation comprises a pharmaceutically acceptable excipient. In some embodiments, the excipient includes a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof.

In some embodiments, the excipient is a solvent. In some embodiments, the solvent is water, dichloromethane, dimethyl sulfoxide, dimethyl formamide, ethyl acetate, methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, or a combination thereof.

In some embodiments, the excipient is a diluent. In some embodiments, the diluent is calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, saline, dry starch, cornstarch, powdered sugar, or a combination thereof.

In some embodiments, the excipient is a surfactant. In some embodiments, the surfactant is polyvinylpyrrolidone (PVP), sodium carboxymethyl cellulose (CMC Na), PEG 4000, TWEEN®20 (polysorbate 20), TWEEN®80 (polysorbate 80), POLOXAMER®188, POLOXAMER®407, monooleate [SPAN®80], or a combination thereof.

In some embodiments, the excipient is a polymer. In some embodiments, the polymer is poly(urethane), poly(siloxane), poly(silicone), poly(methyl methacrylate), poly(vinyl alcohol), poly(ethylene), poly(vinyl pyrrolidone) (PVP), poly(vinyl pyrrolidone) K12 (e.g., PVP K12, polyvidone, or povidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactide (PLA), polyglycolide (PGA), poly(lactide-co-glycolide) (PLGA), polyanhydrides, polyorthoester, and/or a combination thereof.

In some embodiments, the excipient is a cryoprotectant. In some embodiments, the cryoprotectant is mannitol, glycerol, propylene glycol, dimethyl sulfoxide (DMSO), ethylene glycol, 2-methyl-2,4-pentadiol (MPD) or hexylene glycol, sucrose, trehalose, or a combination thereof.

In some embodiments, a formulation can comprise a percentage by weight of an excipient. In some embodiments, a formulation of the present disclosure comprises 5% excipient by weight of the total mass of the formulation. In some embodiments, a formulation of the present disclosure comprises 10% excipient by weight of the total mass of the formulation. In some embodiments, a formulation of the present disclosure comprises 15% excipient by weight of the total mass of the formulation. In some embodiments, a formulation of the present disclosure comprises 20% excipient by weight of the total mass of the formulation. In some embodiments, a formulation of the present disclosure comprises 25% excipient by weight of the total mass of the formulation. In some embodiments, a formulation of the present disclosure comprises 30% excipient by weight of the total mass of the formulation. In some embodiments, a formulation of the present disclosure comprises 35% excipient by weight of the total mass of the formulation. In some embodiments, a formulation of the present disclosure comprises 40% excipient by weight of the total mass of the formulation. In some embodiments, a formulation of the present disclosure comprises 45% excipient by weight of the total mass of the formulation. In some embodiments, a formulation of the present disclosure comprises 50% excipient by weight of the total mass of the formulation. In some embodiments, a formulation of the present disclosure comprises 55% excipient by weight of the total mass of the formulation. In some embodiments, a formulation of the present disclosure comprises 60% excipient by weight of the total mass of the formulation. In some embodiments, a formulation of the present disclosure comprises 65% excipient by weight of the total mass of the formulation. In some embodiments, a formulation of the present disclosure comprises 70% excipient by weight of the total mass of the formulation. In some embodiments, a formulation of the present disclosure comprises 75% excipient by weight of the total mass of the formulation. In some embodiments, a formulation of the present disclosure comprises 80% excipient by weight of the total mass of the formulation. In some embodiments, a formulation of the present disclosure comprises 85% excipient by weight of the total mass of the formulation. In some embodiments, a formulation of the present disclosure comprises 90% excipient by weight of the total mass of the formulation. In some embodiments, a formulation of the present disclosure comprises 95% excipient by weight of the total mass of the formulation.

### Dosages

The dosage (e.g., effective amount) of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative and/or effective amounts of a compound, a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the compound.

In the context of treating cancer or maintaining a reduction in levels of protein expression in a subject, the effective amount of a compound of the present disclosure or a pharmaceutically acceptable salt thereof may be an amount sufficient to reduce the level of protein expression in the subject.

In some embodiments, the reduction in the level of protein expression in the subject is compared to the level of protein expression in the subject prior to administration of the compound of the present disclosure or pharmaceutically acceptable salt thereof. In some embodiments, the reduction in the level of protein expression in the subject is compared to the level of protein expression in a reference. In some embodiments, the reduction in the level of protein expression in the subject is compared to the level of protein expression in a subject who was not administered the compound of the present disclosure or pharmaceutically acceptable salt thereof.

The level of protein expression may be determined in a blood sample of the subject. Accordingly, in some embodiments, the effective amount of a compound of the present disclosure or a pharmaceutically acceptable salt thereof is an amount sufficient to reduce the level of protein expression in a blood sample of the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the level of protein expression in a blood sample of the subject prior to administration of the compound of the present disclosure or pharmaceutically acceptable salt thereof. Accordingly, in some embodiments, the effective amount of a compound of the present disclosure or a pharmaceutically acceptable salt thereof is an amount sufficient to reduce the level of protein expression in a blood sample of the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the level of protein expression in a blood sample of the subject prior to administration of the compound of the present disclosure or pharmaceutically acceptable salt thereof.

The level of protein expression may also be determined in a tumor of the subject. Accordingly, in some embodiments, the effective amount of a compound of the present disclosure or a pharmaceutically acceptable salt thereof is an amount sufficient to reduce the level of protein expression in a tumor of the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the level of protein expression in the tumor the subject prior to administration of the compound of the present disclosure or pharmaceutically acceptable salt thereof. Accordingly, in some embodiments, the effective amount of a compound of the present disclosure or a pharmaceutically acceptable salt thereof is an amount sufficient to reduce the level of protein expression in a tumor of the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the level of protein expression in the tumor the subject prior to administration of the compound of the present disclosure or pharmaceutically acceptable salt thereof.

In some embodiments, the effective amount of a compound of the present disclosure or a pharmaceutically acceptable salt thereof is an amount sufficient to maintain a reduction of protein expression of between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) in the subject over at least 14 days (e.g., at least 21 days or at least 28 days). In some embodiments, the effective amount of a compound of the present disclosure or a pharmaceutically acceptable salt thereof is an amount sufficient to maintain a reduction of protein expression of at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) in the subject over at least one week (e.g., at least two weeks, at least three weeks, at least four weeks, at least six weeks, at least eight weeks, at least one month, or at least two months).

In some embodiments, the effective amount of a compound of the present disclosure or a pharmaceutically acceptable salt thereof is administered in a dose of between about 0.1 mg/kg and about 1000 mg/kg.

In some embodiments, the effective amount of a compound of the present disclosure or a pharmaceutically acceptable salt thereof is administered in a dose of between about 0.1 mg/mL and about 1000 mg/mL.

In some embodiments, the effective amount of a compound of the present disclosure or a pharmaceutically acceptable salt thereof is administered in a dose of between about 0.1 mg and about 1000 mg.

In some embodiments, a formulation of the present disclosure is administered in a dose volume of between about 0.25 mL and about 3 mL. In some embodiments, a formulation of the present disclosure is administered in a dose volume of between about 0.50 mL and about 3 mL. In some embodiments, a formulation of the present disclosure is administered in a dose volume of between about 0.75 mL and about 3 mL. In some embodiments, a formulation of the present disclosure is administered in a dose volume of between about 1 mL and about 3 mL. In some embodiments, a formulation of the present disclosure is administered in a dose volume of between about 1 mL and about 2 mL. In some embodiments, a formulation of the present disclosure is administered in a dose volume of between about 1.5 mL and about 3 mL. In some embodiments, a formulation of the present disclosure is administered in a dose volume of between about 2 mL and about 3 mL.

In some embodiments, the effective amount of a compound of the present disclosure or a pharmaceutically acceptable salt thereof is administered in a dose of between about 0.1 mg/m² and about 1000 mg/m². In some embodiments, the effective amount of a compound of the present disclosure or a pharmaceutically acceptable salt thereof is administered at least once per week. In some embodiments, the effective amount of a compound of the present disclosure or a pharmaceutically acceptable salt thereof is administered at least once every two weeks. In some embodiments, the effective amount of a compound of the present disclosure or a pharmaceutically acceptable salt thereof is administered at least once every three weeks. In some embodiments, the effective amount of a compound of the present disclosure or a pharmaceutically acceptable salt thereof is administered at least once every four weeks.

In some embodiments, the effective amount of a compound of the present disclosure or a pharmaceutically acceptable salt thereof is administered at least once every six weeks. In some embodiments, the effective amount of a compound of the present disclosure or a pharmaceutically acceptable salt thereof is administered at least once every eight weeks. In some embodiments, the effective amount of a compound of the present disclosure or a pharmaceutically acceptable salt thereof is administered at least once every month. In some embodiments, the effective amount of a compound of the present disclosure or a pharmaceutically acceptable salt thereof is administered at least once every two months.

In some embodiments, a formulation of the present disclosure is administered at least once every week. In some embodiments, a formulation of the present disclosure is administered at least once every two weeks. In some embodiments, a formulation of the present disclosure is administered at least once every three weeks. In some embodiments, a formulation of the present disclosure is administered at least once every four weeks. In some embodiments, a formulation of the present disclosure is administered at least once every six weeks. In some embodiments, a formulation of the present disclosure is administered at least once every eight weeks. In some embodiments, a formulation of the present disclosure is administered at least once every month. In some embodiments, a formulation of the present disclosure is administered at least once every two months.

In some embodiments, the effective amount of a compound of the present disclosure or a pharmaceutically acceptable salt thereof is administered to a subject in a 7-day dosing cycle. In some embodiments, the effective amount of a compound of the present disclosure or a pharmaceutically acceptable salt thereof is administered to a subject in a one-week dosing cycle. In some embodiments, the effective amount of a compound of the present disclosure or a pharmaceutically acceptable salt thereof is administered to a subject in a 14-day dosing cycle. In some embodiments, the effective amount of a compound of the present disclosure or a pharmaceutically acceptable salt thereof is administered to a subject in a two-week dosing cycle. In some embodiments, the effective amount of a compound of the present disclosure or a pharmaceutically acceptable salt thereof is administered to a subject in a 21-day dosing cycle. In some embodiments, the effective amount of a compound of the present disclosure or a pharmaceutically acceptable salt thereof is administered to a subject in a three-week dosing cycle. In some embodiments, the effective amount of a compound of the present disclosure or a pharmaceutically acceptable salt thereof is administered to a subject in a 28-day dosing cycle. In some embodiments, the effective amount of a compound of the present disclosure or a pharmaceutically acceptable salt thereof is administered to a subject in a four-week dosing cycle. In some embodiments, the effective amount of a compound of the present disclosure or a pharmaceutically acceptable salt thereof is administered to a subject in a 42-day dosing cycle. In some embodiments, the effective amount of a compound of the present disclosure or a pharmaceutically acceptable salt thereof is administered to a subject in a six-week dosing cycle. In some embodiments, the effective amount of a compound of the present disclosure or a pharmaceutically acceptable salt thereof is administered to a subject in a 56-day dosing cycle. In some embodiments, the effective amount of a compound of the present disclosure or a pharmaceutically acceptable salt thereof is administered to a subject in an eight-week dosing cycle. In some embodiments, the effective amount of a compound of the present disclosure or a pharmaceutically acceptable salt thereof is administered to a subject in a one-month dosing cycle. In some embodiments, the effective amount of a compound of the present disclosure or a pharmaceutically acceptable salt thereof is administered to a subject in a two-month dosing cycle.

In some embodiments, a formulation of the present disclosure is administered to a subject in a 7-day dosing cycle. In some embodiments, a formulation of the present disclosure is administered to a subject in a one-week dosing cycle. In some embodiments, a formulation of the present disclosure is administered to a subject in a 14-day dosing cycle. In some embodiments, a formulation of the present disclosure is administered to a subject in a two-week dosing cycle. In some embodiments, a formulation of the present disclosure is administered to a subject in a 21-day dosing cycle. In some embodiments, a formulation of the present disclosure is administered to a subject in a three-week dosing cycle. In some embodiments, a formulation of the present disclosure is administered to a subject in a 28-day dosing cycle. In some embodiments, a formulation of the present disclosure is administered to a subject in a four-week dosing cycle. In some embodiments, a formulation of the present disclosure is administered to a subject in a 42-day dosing cycle. In some embodiments, a formulation of the present disclosure is administered to a subject in a six-week dosing cycle. In some embodiments, a formulation of the present disclosure is administered to a subject in a 56-day dosing cycle. In some embodiments, a formulation of the present disclosure is administered to a subject in an eight-week dosing cycle. In some embodiments, a formulation of the present disclosure is administered to a subject in a one-month dosing cycle. In some embodiments, a formulation of the present disclosure is administered to a subject in a two-month dosing cycle.

### Combination Therapies

Methods of the present disclosure may include administering to the subject in need thereof an second therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Also included is 5-fluorouracil (5-FU), leucovorin (LV), irinotecan, oxaliplatin, capecitabine, paclitaxel, and docetaxel. Non-limiting examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolmelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomycin, actinomycin, authramycin, azaserine, bleomycin, cactinomycin, carubicin, carzinophilin, chromomycin, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocin; mitoguazone; mitoxantrone; mopidamol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verrucarin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® docetaxel (Rhone-Poulenc Rorer, Antony, France); chlorambucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a DNA damaging agent (e.g., a platinum-based antineoplastic agent, topoisomerase inhibitors, PARP inhibitors, alkylating antineoplastic agents, and ionizing radiation).

Examples of platinum-based antineoplastic agent that may be used as a second therapeutic agent in the compositions and methods of the disclosure are cisplatin, carboplatin, oxaliplatin, dicycloplatin, eptaplatin, lobaplatin, miriplatin, nedaplatin, triplatin tetranitrate, phenanthriplatin, picoplatin, and satraplatin. In some embodiments, the second therapeutic agent is cisplatin and the treated cancer is a testicular cancer, ovarian cancer, or a bladder cancer (e.g., advanced bladder cancer). In some embodiments, the second therapeutic agent is carboplatin and the treated cancer is an ovarian cancer, lung cancer, head and neck cancer, brain cancer, or neuroblastoma. In some embodiments, the second therapeutic agent is oxaliplatin and the treated cancer is a colorectal cancer. In some embodiments, the second therapeutic agent is dicycloplatin and the treated cancer is a non-small cell lung cancer or prostate cancer. In some embodiments, the second therapeutic agent is eptaplatin and the treated cancer is a gastric cancer. In some embodiments, the second therapeutic agent is lobaplatin and the treated cancer is a breast cancer. In some embodiments, the second therapeutic agent is miriplatin and the treated cancer is a hepatocellular carcinoma. In some embodiments, the second therapeutic agent is nedaplatin and the treated cancer is a nasopharyngeal carcinoma, esophageal cancer, squamous cell carcinoma, or cervical cancer. In some embodiments, the second therapeutic agent is triplatin tetranitrate and the treated cancer is a lung cancer (e.g., small cell lung cancer) or pancreatic cancer. In some embodiments, the second therapeutic agent is picoplatin and the treated cancer is a lung cancer (e.g., small cell lung cancer), prostate cancer, bladder cancer, or colorectal cancer. In some embodiments, the second therapeutic agent is satraplatin and the treated cancer is a prostate cancer, breast cancer, or lung cancer.

Examples of topoisomerase inhibitors that may be used as a second therapeutic agent in the compositions and methods of the disclosure are etoposide, teniposide, doxorubicin, daunorubicin, mitoxantrone, amsacrine, ellipticine, irinotecan, topotecan, camptothecin, and diflomotecan. In some embodiments, the second therapeutic agent is etoposide and the treated cancer is a lung cancer (e.g., small cell lung cancer) or testicular cancer. In some embodiments, the second therapeutic agent is teniposide and the treated cancer is an acute lymphoblastic leukemia (e.g., childhood acute lymphoblastic leukemia). In some embodiments, the second therapeutic agent is doxorubicin and the treated cancer is an acute lymphoblastic leukemia, acute myeloblastic leukemia, Hodgkin's lymphoma, non-Hodgkin's lymphoma, breast cancer, Wilms tumor, neuroblastoma, soft tissue sarcoma, bone sarcomas, ovarian carcinoma, transitional cell bladder carcinoma, thyroid carcinoma, gastric carcinoma, or bronchogenic carcinoma. In some embodiments, the second therapeutic agent is daunorubicin and the treated cancer is an acute lymphoblastic leukemia or acute myeloid leukemia. In some embodiments, the second therapeutic agent is mitoxantrone and the treated cancer is a prostate cancer or acute nonlymphocytic leukemia. In some embodiments, the second therapeutic agent is amsacrine and the treated cancer is a leukemia (e.g., acute adult leukemia). In some embodiments, the second therapeutic agent is irinotecan and the treated cancer is a colorectal cancer. In some embodiments, the second therapeutic agent is topotecan and the treated cancer is a lung cancer (e.g., small cell lung cancer). In some embodiments, the second therapeutic agent is diflomotecan and the treated cancer is a lung cancer (e.g., small cell lung cancer).

Examples of alkylating antineoplastic agents that may be used as a second therapeutic agent in the compositions and methods of the disclosure are cyclophosphamide, uramustine, melphalan, chlorambucil, ifosfamide, bendamustine, carmustine, lomustine, chlorozotocin, fotemustine, nimustine, ranimustine, busulfan, improsulfan, piposulfan, chlornaphazine, cyclophosphamide, estramustine, mechlorethamine, mechlorethamine oxide hydrochloride, novembichin, phenesterine, prednimustine, trofosfamide, procarbazine, altretamine, dacarbazine, mitozolomide, and temozolomide. In some embodiments, the second therapeutic agent is cyclophosphamide and the treated cancer is a non-Hodgkin's lymphoma. In some embodiments, the second therapeutic agent is melphalan and the treated cancer is a multiple myeloma, ovarian cancer, or melanoma. In some embodiments, the second therapeutic agent is chlorambucil and the treated cancer is a chronic lymphatic leukemia, malignant lymphoma (e.g., lymphosarcoma, giant follicular lymphoma, or Hodgkin's lymphoma). In some embodiments, the second therapeutic agent is ifosfamide and the treated cancer is a testicular cancer. In some embodiments, the second therapeutic agent is bendamustine and the treated cancer is a chronic lymphocytic leukemia or non-Hodgkin's lymphoma. In some embodiments, the second therapeutic agent is carmustine and the treated cancer is a brain cancer (e.g., glioblastoma, brainstem glioma, medulloblastoma, astrocytoma, ependymoma, or a metastatic brain tumor), multiple myeloma, Hodgkin's disease, or non-Hodgkin's lymphoma. In some embodiments, the second therapeutic agent is lomustine and the treated cancer is a brain cancer or Hodgkin's lymphoma. In some embodiments, the second therapeutic agent is fotemustine and the treated cancer is a melanoma. In some embodiments, the second therapeutic agent is nimustine and the treated cancer is a brain cancer. In some embodiments, the second therapeutic agent is ranimustine and the treated cancer is a chronic myelogenous leukemia or polycythemia vera. In some embodiments, the second therapeutic agent is busulfan and the treated cancer is a chronic myelogenous leukemia. In some embodiments, the second therapeutic agent is improsulfan and the treated cancer is a sarcoma. In some embodiments, the second therapeutic agent is estramustine and the treated cancer is a prostate cancer (e.g., prostate carcinoma). In some embodiments, the second therapeutic agent is mechlorethamine and the treated cancer is a cutaneous T-cell lymphoma. In some embodiments, the second therapeutic agent is trofosfamide and the treated cancer is a sarcoma (e.g., soft tissue sarcoma). In some embodiments, the second therapeutic agent is procarbazine and the treated cancer is a Hodgkin's disease. In some embodiments, the second therapeutic agent is altretamine and the treated cancer is an ovarian cancer. In some embodiments, the second therapeutic agent is dacarbazine and the treated cancer is a melanoma, Hodgkin's lymphoma, or sarcoma. In some embodiments, the second therapeutic agent is temozolomide and the treated cancer is a brain cancer (e.g., astrocytoma or glioblastoma) or lung cancer (e.g., small cell lung cancer).

Examples of PARP inhibitors that may be used as a second therapeutic agent in the compositions and methods of the disclosure are niraparib, olaparib, rucaparib, talazoparib, veliparib, pamiparib, CK-102, or E7016. Advantageously, the compounds of the disclosure and a DNA damaging agent may act synergistically to treat cancer. In some embodiments, the second therapeutic agent is niraparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is olaparib and the treated cancer is a lung cancer (e.g., small cell lung cancer), ovarian cancer (e.g., BRCA mutated ovarian cancer), breast cancer (e.g., BRCA mutated breast cancer), fallopian tube cancer (e.g., BRCA mutated fallopian tube cancer), primary peritoneal cancer (e.g., BRCA mutated primary peritoneal cancer), prostate cancer (e.g., castration-resistant prostate cancer), or pancreatic cancer (e.g., pancreatic adenocarcinoma). In some embodiments, the second therapeutic agent is rucaparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is talazoparib and the treated cancer is a breast cancer (e.g., BRCA mutated breast cancer). In some embodiments, the second therapeutic agent is veliparib and the treated cancer is a lung cancer (e.g., non-small cell lung cancer), melanoma, breast cancer, ovarian cancer, prostate cancer, or brain cancer. In some embodiments, the second therapeutic agent is pamiparib and the treated cancer is an ovarian cancer. In some embodiments, the second therapeutic agent is CK-102 and the treated cancer is a lung cancer (e.g., non-small cell lung cancer). In some embodiments, the second therapeutic agent is E7016 and the treated cancer is a melanoma.

Without wishing to be bound by theory, the synergy between the compounds of the disclosure and DNA damaging agents may be attributed to the necessity of BRD9 for DNA repair; inhibition of BRD9 may sensitize cancer (e.g., cancer cell or cancer tissue) to DNA damaging agents.

In some embodiments, the second therapeutic agent is a JAK inhibitor (e.g., JAK1 inhibitor). Non-limiting examples of JAK inhibitors that may be used as a second therapeutic agent in the compositions and methods of the disclosure include tofacitinib, ruxolitinib, oclacitinib, baricitinib, peficitinib, fedratinib, upadacitinib, filgotinib, cerdulatinib, gandotinib, lestaurtinib, momelotinib, pacritinib, abrocitinib, solcitinib, itacitinib, or SHR0302. Without wishing to be bound by theory, the synergy between the compounds of the disclosure and JAK inhibitors may be inhibitor of SAGA complex to their combined effect of downregulating Foxp3+ Treg cells. In some embodiments, the second therapeutic agent is ruxolitinib and the treated cancer is a myeloproliferative neoplasm (e.g., polycythemia or myelofibrosis), ovarian cancer, breast cancer, pancreatic cancer. In some embodiments, the second therapeutic agent is fedratinib and the treated cancer is a myeloproliferative neoplasm (e.g., myelofibrosis). In some embodiments, the second therapeutic agent is cerdulatinib and the treated cancer is a lymphoma (e.g., peripheral T-cell lymphoma). In some embodiments, the second therapeutic agent is gandotinib and the treated cancer is a myeloproliferative neoplasm (e.g., polycythemia or myelofibrosis). In some embodiments, the second therapeutic agent is lestaurtinib and the treated cancer is a myeloproliferative neoplasm (e.g., polycythemia or myelofibrosis), leukemia (e.g., acute myeloid leukemia), pancreatic cancer, prostate cancer, or neuroblastoma. In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma). In some embodiments, the second therapeutic agent is momelotinib and the treated cancer is a myeloproliferative neoplasm (e.g., polycythemia or myelofibrosis). In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma).

In some embodiments, the second therapeutic agent is an inhibitor of SAGA complex or a component thereof. A SAGA complex inhibitor may be, e.g., an inhibitory antibody or small molecule inhibitor, of CCDC101, Tada2B, Tada3, Usp22, Tada1, Taf6I, Supt5, Supt20, or a combination thereof.

Without wishing to be bound by theory, the synergy between the compounds of the disclosure and inhibitors of SAGA complex may be attributed to their combined effect of downregulating Foxp3+ Treg cells. In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the second therapeutic agent is ipilimumab and the treated cancer is a melanoma, kidney cancer, lung cancer (e.g., non-small cell lung cancer or small cell lung cancer), or prostate cancer. In some embodiments, the second therapeutic agent is tremelimumab and the treated cancer is a melanoma, mesothelioma, or lung cancer (e.g., non-small cell lung cancer). In some embodiments, the second therapeutic agent is nivolumab and the treated cancer is a melanoma, lung cancer (e.g., non-small cell lung cancer or small cell lung cancer), kidney cancer, Hodgkin's lymphoma, head and neck cancer (e.g., squamous cell carcinoma of the head and neck), urothelial carcinoma, hepatocellular carcinoma, or colorectal cancer. In some embodiments, the second therapeutic agent is pembrolizumab and the treated cancer is a melanoma, lung cancer (e.g., non-small cell lung cancer or small cell lung cancer), Hodgkin's lymphoma, head and neck cancer (e.g., squamous cell carcinoma of the head and neck), primary mediastinal large B-cell lymphoma, urothelial carcinoma, hepatocellular carcinoma, microsatellite instability-high cancer, gastric cancer, esophageal cancer, cervical cancer, Merkel cell carcinoma, kidney carcinoma, or endometrial carcinoma. In some embodiments, the second therapeutic agent is MPDL3280A and the treated cancer is a lung cancer (e.g., non-small cell lung cancer or small cell lung cancer), urothelial carcinoma, hepatocellular carcinoma, or breast cancer. In some embodiments, the second therapeutic agent is MEDI4736 and the treated cancer is a lung cancer (e.g., non-small cell lung cancer or small cell lung cancer) or urothelial carcinoma. In some embodiments, the second therapeutic agent is MSB0010718C and the treated cancer is a urothelial carcinoma. In some embodiments, the second therapeutic agent is MSB0010718C and the treated cancer is a melanoma, lung cancer (e.g., non-small cell lung cancer), colorectal cancer, kidney cancer, ovarian cancer, pancreatic cancer, gastric cancer, and breast cancer.

Advantageously, the compounds of the disclosure and a checkpoint inhibitor may act synergistically to treat cancer. Without wishing to be bound by theory, the synergy between the compounds of the disclosure and checkpoint inhibitors may be attributed to the checkpoint inhibitor efficacy enhancement associated with the BRD9 inhibition-induced downregulation of Foxp3+ Treg cells.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the present disclosure or pharmaceutically acceptable salts thereof are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the disclosure, the described compounds or salts, solvates, or prodrugs thereof may be administered to a patient in a variety of forms depending on the selected route of administration, as will be understood by those skilled in the art. The compounds described herein may be administered, for example, by parenteral administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. In particular embodiments, a compound of the present disclosure or a pharmaceutically acceptable salt thereof is formulated for intramuscular and/or subcutaneous administration. In some embodiments, a formulation of the present disclosure is administered to a subject in need thereof intramuscularly. In some embodiments, a formulation of the present disclosure is administered to a subject in need thereof subcutaneously.

Formulations of a compound described herein can be prepared in combination with a suitable excipient, such as a surfactant, a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof. Dispersions can be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

In some embodiments, a formulation of the present disclosure is administered using an 18 gauge needle. In some embodiments, a formulation of the present disclosure is administered using a 19 gauge needle. In some embodiments, a formulation of the present disclosure is administered using a 20 gauge needle. In some embodiments, a formulation of the present disclosure is administered using a 21 gauge needle. In some embodiments, a formulation of the present disclosure is administered using a 22 gauge needle. In some embodiments, a formulation of the present disclosure is administered using a 23 gauge needle. In some embodiments, a formulation of the present disclosure is administered using a 24 gauge needle. In some embodiments, a formulation of the present disclosure is administered using a 25 gauge needle. In some embodiments, a formulation of the present disclosure is administered using a 26 gauge needle. In some embodiments, a formulation of the present disclosure is administered using a 27 gauge needle. In some embodiments, a formulation of the present disclosure is administered using a 28 gauge needle. In some embodiments, the weight of a subject in need thereof determines the needle gauge used for administration. The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

In some embodiments, the administering comprises combining the compound with an excipient to form a solution and administering the resulting solution.

In some embodiments, the administering comprises coupling a vial comprising the compound and a syringe comprising an excipient, transferring the contents of the syringe into the vial, mixing to form a solution, and administering the resulting solution.

In some embodiments, the administering comprises coupling a first syringe comprising the compound and a second syringe comprising an excipient, mixing to form a solution, and administering the resulting solution.

In some embodiments, the administering comprises coupling a first vial comprising the compound and a second vial comprising an excipient, mixing to form a solution, and administering the resulting solution.

In some embodiments, the administering comprises a syringe comprising the compound and one or more excipients, mixing to form a solution, and administering the resulting solution.

In some embodiments, the administering comprises coupling a vial comprising the compound and a syringe comprising PLGA and an excipient, transferring the contents of the syringe into the vial, mixing to form a solution, and administering the resulting solution.

In some embodiments, the administering comprises coupling a syringe comprising the compound and a syringe comprising PLGA and an excipient, mixing to form a solution, and administering the resulting solution.

In some embodiments, the administering comprises mixing the formulation to form a suspension and administering the resulting suspension.

In some embodiments, the administering comprises mixing the formulation to form a nanosuspension and administering the resulting nanosuspension.

In some embodiments, the administering comprises mixing the formulation to form a microsuspension and administering the resulting microsuspension.

In some embodiments, the administering comprises mixing the formulation to form a PLGA microsphere solution and administering the resulting solution.

In some embodiments, the administering comprises mixing the formulation to form an in situ PLGA gel solution and administering the resulting solution.

In some embodiments, the administering comprises mixing the formulation to form an SAIB nanosuspension and administering the resulting nanosuspension.

In some embodiments, the administering comprises mixing the formulation to form an in situ SAIB gel solution and administering the resulting solution.

### EXAMPLES

### Example 1: Nanosuspension and Microsuspension Formulations of Compound A

Generally, nanosuspension formulations of Compound A (e.g., 5 mg/ml to 150 mg/ml concentration range) of suitable particle size distribution (e.g., D50 ranging from 1 micron to <500 nm) were prepared using a low energy jar mill (e.g., at 250 rpm or higher for an optimum period of time such as 4 hours) or a high energy mill (e.g., at 1000 rpm or higher for 15-90 minutes) using polymeric or zirconium dioxide beads of 0.3 mm-1 mm size. Suspensions were also obtained by jet milling the drug substance to suitable particle size and using an appropriate vehicle such as low viscosity CMC Na for suspending.

More specifically, nanosuspensions were prepared using two different methods as described below.

### Nanosuspension Preparation Method 1:

Milling was performed in a high energy ball mill with suitable sized-spherical, polymeric or Zirconium oxide beads. Compound A and excipient(s) as indicated in Table 1 were combined in deionized water per the indicated batch size and transferred to the milling chamber. Nanosuspensions A through D were generated utilizing process conditions denoted in Table 2.

### Nanosuspension Preparation Method 2:

Compound A was first mechanically jet-milled to reduce initial particle size prior to ball milling. The jet mill feed pressure was 4.5 bar and milling pressure was 5.0 bar. The resulting particle size of jet milled Compound A was 5-6 microns (050). Jet-milled Compound A was combined with an aqueous solution of excipient(s) as described in Table 3 and combined with ball milling media (e.g., 0.3 mm diameter ZrO2 beads) to prepare for ball milling. A High-Capacity Laboratory jar mill was used to create nanosuspensions E through G using the conditions listed in Table 4. Formulation EF1 is a dilution of the 40 mg/mL formulation of E or F. Solid state of each formulation was crystalline via XRPD.

TABLE 3
Nanosuspension Formulation Compositions

| | Batch | Com- | | |
|---|---|---|---|---|
| For- | Size | pound A | Excipient 1 | Excipient 2 |
| mulation | (g) | (wt %) | (wt %) | (wt %) |
| A | 6.0 | 20 | 2 wt % TWEEN ®20 | — |
| B | 6.2 | 10 | 1.5 wt % | — |
| | | | TWEEN ®20 | |
| C | 6.2 | 15 | 2.25 wt % | — |
| | | | TWEEN ®20 | |
| D | 6.2 | 10 | 1 wt % TWEEN ®20 | 0.5 wt % |
| | | | | poly(vinyl |
| | | | | pyrrolidone) |
| E | 35 | 4.4 | 0.5 wt % CMC Na¹ | 0.2 wt % |
| | | | | TWEEN ®20 |
| F | 17 | 4.2 | 0.5 wt % CMC Na | 0.2 wt % |
| | | | | TWEEN ®20 |
| G | 15 | 10.8 | 0.5 wt % CMC Na | 0.2 wt % |
| | | | | TWEEN ®20 |
| EF1 | — | 0.4-0.5% | 0.5 wt % CMC Na | 0.2 wt % |
| | | | | TWEEN ®20 |

¹CMC Na = sodium carboxymethyl cellulose

TABLE 4
Example Nanosuspension Formulation Milling Parameters

| | Preparation | Milling | Mill | Milling | Milling |
|---|---|---|---|---|---|
| For- | Method | Media, | Speed, | Time | Temperature |
| mulation | Used | (vol %) | (RPM) | (min) | (° C.) |
| A | 1 | 85¹ | 2500 | 30 min | 5° C. |
| B | 1 | 65¹ | 2500 | 40 min | 5° C. |
| C | 1 | 65¹ | 2500 | 40 min | 5° C. |
| D | 1 | 65¹ | 2500 | 25 min | 5° C. |
| E | 2 | 1.5² | 300 | 13 hr | RT³ |
| F | 2 | 1.5² | 300 | 15 hr | RT |
| G | 2 | 1.5² | 300 | 22 hr | RT |

¹Calculated as volume of the milling chamber.
²Calculated as multiplier of batch size.
³Room Temperature

Other processing conditions and milling media, not represented here, can also be implemented for generating nanosuspensions of Compound A.

Nanosuspensions were characterized with respect to particle size, Compound A concentration, purity, and XRPD (X-Ray Powder Diffraction) (Table 5). As demonstrated by particle size analysis, the milling processes were effective in generating nanoparticles, with a majority of particles measuring at less than 1 micron. Concentrations of Compound A measuring from approximately 4 mg/mL to about 200 mg/mL were achieved.

TABLE 5
Nanosuspension Formulation Characterization Results

| | Particle | Compound A | |
|---|---|---|---|
| | Size D(50), | Concentration, | |
| Formulation | (microns) | (mg/mL) | XRPD |
| A | 0.138 | 200 | Crystalline |
| B | 0.131 | 100 | peaks |
| C | 0.135 | 150 | observed, |
| D | 0.135 | 100 | no form |
| E | 0.273 | 39.2 | change |
| F | 0.309 | 36.1 | |
| G | 0.450 | 91.0 | |
| EF1 | 0.302 | 3.7 mg/ml | |

A microsuspension of jet-milled drug-substance of Compound A was also prepared (Formulation H). Approximately 3 g of Compound A was subjected to a jet mill using a feed pressure of 6 bar and milling pressure of 5 bar. The feed rate was 1.52 g/10 min at this scale. The particle size of formulation H was D50=3.48 microns and D90=8.47 microns. Solid state of the formulation was crystalline via XRPD. The milled material showed comparable physical and chemical properties to the original form of Compound A, including solid form, purity, water uptake.

### Nanosuspension and Microsuspension Rat PK Study

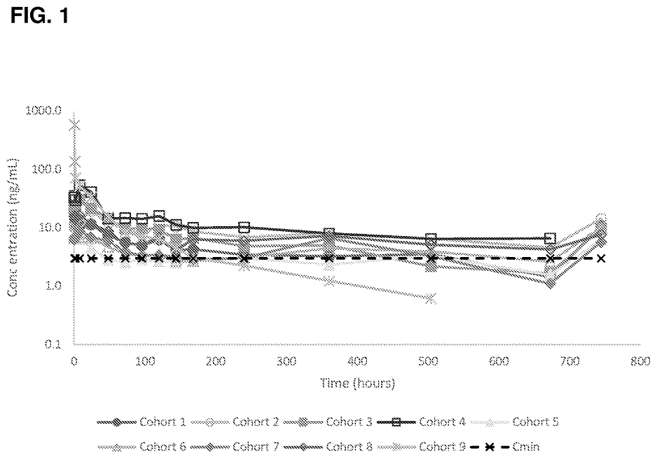

Performance of nanosuspensions E/F and G, and microsuspension H were evaluated in Male SD rats. Note that formulations E and F were used interchangeably in this experiment as they had almost identical properties (see Tables 3-5), which is denoted herein as “E/F”. The animals were approximately 6-8 weeks of age and approximately 200-300 g at time of dosing. An IV formulation (denoted as Formulation IV) was used as a control (20% SBE-CD in 5 mM citrate buffer pH3, final pH adjusted to a pH of 5 by using 1 N NaOH). The nanosuspensions were dosed at 20 mg/kg and 40 mg/kg and the microsuspension was dosed at 40 mg/kg in saline (Table 6). Both subcutaneous (SC) and intramuscular (IM) routes of administration were evaluated. At each time point, approximately 0.2 mL blood was collected via jugular vein and analyzed by LC-MS/MS for mean plasma concentration of Compound A (FIG. 1). Pharmacokinetics determined that a target Cmin of 3 ng/mL was desired, which is denoted by the black dashed line in FIG. 1.

TABLE 6
Rat PK Study Parameters of Nanosuspension and
Microsuspension Formulations

| | | | Initial | Dose | Route |
|---|---|---|---|---|---|
| | Legend | | concentration | admin- | of |
| | in | For- | of Formulation | istered | admin- |
| Cohort | FIG. 1 | mulation | (mg/mL) | (mg/kg) | istration |
| 1 | ● | E/F | 40 | 20 | SC |
| 2 | ◯ | E/F | 40 | 40 | SC |
| 3 | ▪ | E/F | 40 | 20 | IM |
| 4 | □ | G | 100 | 40 | IM |
| 5 | ▴ | H | 40 | 20 | SC |
| 6 | Δ | H | 40 | 40 | SC |
| 7 | ♦ | H | 40 | 20 | IM |
| 8 | ⋄ | H | 40 | 40 | IM |
| 9 | X | IV | — | 8 | IV |

FIG. 1 shows that nanosuspensions E-G and microsuspension H met the target Cmin criteria of 3 ng/ml over a course of two weeks (336 hours). Further, at the same dose level, IM and SC routes of administration of these formulations were comparable at two weeks, and exposure is dose proportional at terminal time-point(s). At a dose of 40 mg/kg, nanosuspensions E-G and microsuspension H met m criteria of 3 ng/mL for at least 4 weeks (672 hours). These data suggest that nanosuspension and microsuspension formulations of Compound A are a good format for long acting or sustained drug release, and for maintaining a target plasma concentration of 3 ng/mL over a dosing cycle.

### Nanosuspension Mouse PK/PD Study

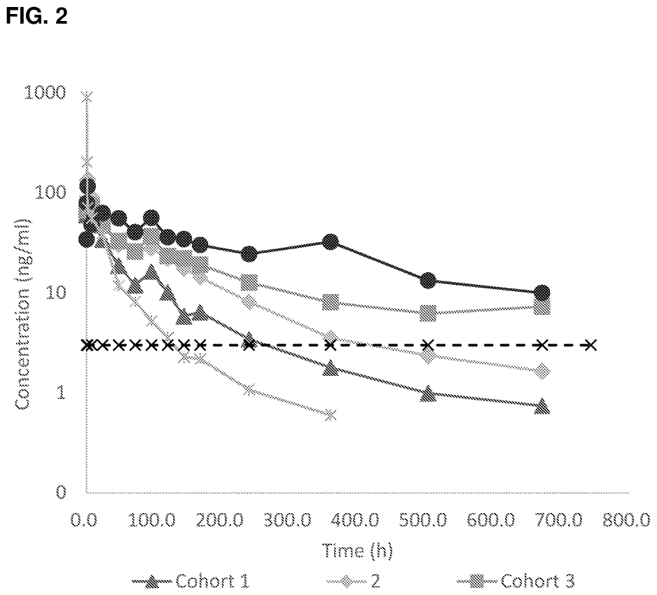

Performance of nanosuspensions E/F and EF1 were evaluated in Male 001 mice. The animals were approximately 6-8 weeks of (age and weighed approximately 20-30 g at dosing. The nanosuspensions were dosed sub-cutaneously at 8 mg/kg, 30 mg/kg. 90 mg/kg and 200 mg/kg (Table 7). At each time point, approximately 0.03 mL blood was collected via jugular vein and analyzed by LC-MS/MS for mean plasma concentration of Compound A (FIG. 2). Pharmacokinetics determined that a target Cmin of 3 ng/mL was desired, which is denoted by the black dashed line in FIG. 2.

TABLE 7
Mouse PK Study Parameters of Nanosuspension Formulations

| | | | Initial | Dose | Route |
|---|---|---|---|---|---|
| | Legend | | concentration | admin- | of |
| | in | For- | of Formulation | istered | admin- |
| Cohort | FIG. 6 | mulation | (mg/mL) | (mg/kg) | istration |
| 1 | ▴ | EF1 | 5 | 8 | SC |
| | | | (diluted from | | |
| | | | 40 mg/ml | | |
| | | | formulation) | | |
| 2 | ♦ | EF1 | 5 | 30 | SC |
| | | | (diluted from | | |
| | | | 40 mg/mL | | |
| | | | formulation) | | |
| 3 | ▪ | E/F | 40 | 90 | SC |
| 4 | ● | E/F | 40 | 200 | SC |
| 5 | * | IV | — | 8 | IV |

FIG. 2 shows that nanosuspensions E and/or F administered at a dose of 30 mg/kg or higher met the target Cmin criteria of 3 ng/ml over a course of two weeks (336 hours). At a dose of 90 mg/kg or higher, mg/mL, nanosuspensions E and/or F met Cmin criteria of 3 ng/ml for at least 4 weeks (672 hours). Further, exposure is dose proportional at 2 weeks. These data suggest that nanosuspension formulations of Compound A are a good format for long acting or sustained drug release, and for maintaining a target plasma concentration of 3 ng/mL over a dosing cycle.

### Nanosuspension Mouse Efficacy and PK/PD Study

### Efficacy:

Each mouse was inoculated subcutaneously on the right flank with a single cell suspension of SYO-1 human biphasic synovial sarcoma tumor cells (5×10⁶) in 100 μL DMEM with 10% FBS for the tumor development. Treatment (Table 6) was started when mean tumor volume reached about 100-150 mm³ for the efficacy cohorts. Based on the tumor volume, mice were randomly assigned to respective groups using a computer-generated randomization procedure. Corresponding formulations are in Table 6. For each dosing cohort, dosing volume was calculated based on concentration of the formulation and dose volume (ml/kg). Vehicle for IV was 20% HP-β-CD in 5 mM citrate buffer of pH 3, with adjustment to a final PH of 5. Body weight of all animals was measured and recorded twice a week throughout the study. The measurement of tumor size was conducted twice a week with a caliper and recorded. Tumor volume was used for calculation of tumor growth inhibition (TGI) value of the tumors in the treated and the control cohorts, respectively, on a given day after tumor inoculation.

64 plasma samples were collected at two time points (672 h and 676 h) from efficacy study cohorts. 64 tumor samples were collected at two time points (672 h and 676 h) from efficacy cohorts 1-8 and cut into 3 pieces: one piece for PK analysis, one piece for WB analysis (BRD9), and one piece snap frozen for further analysis (n=4). Animals that were observed to be in a continuing deteriorating condition or their tumor size exceeding 2,000 mm³ were euthanized prior to death, or before reaching a comatose state. The entire cohort was terminated when the mean tumor exceeded 2000 mm³ in that cohort.

TABLE 8
Groups and Treatment for Mouse Efficacy Experiment

| | | | | | Formulation & | | | | Collection |
|---|---|---|---|---|---|---|---|---|---|
| | | # Animals/ | Dose | Vol | Initial Conc. | | Dosing | Study | time points |
| Cohort | Drug | cohort | (mg/kg) | (ml/kg) | (mg/mL) | Route | Freq. | Duration | (hr) |
| 1 | Vehicle\hat{ } | 8 | — | 5 | Vehicle\hat{ } | IV | BIW | 4 weeks | 672 |
| 2 | Compound | 8 | 1 | 5 | 0.2 mg/mL of | IV | BIW | 4 weeks | 672 |
| | A | | | | IV* | | | | |
| 3 | Compound | 8 | 1 + 30 | 5 + 6 | 0.2 mg/mL of | IV + SC | 1 dose + 1 | 4 weeks | 672 |
| | A | | | | IV* + | | dose | | |
| | | | | | 5 mg/mL of | | | | |
| | | | | | Formulation | | | | |
| | | | | | EF1 (diluted | | | | |
| | | | | | from 40 mg/mL | | | | |
| | | | | | formulation) | | | | |
| 4 | Compound | 8 | 200 | 5 | 40 mg/ml of | SC | 1 dose | 4 weeks | 672 |
| | A | | | | Formulation | | | | |
| | | | | | E/F | | | | |
| 5 | Compound | 8 | 30 | 6 | 5 mg/mL of | SC | 1 dose | 4 weeks | 672 |
| | A | | | | Formulation | | | | |
| | | | | | E/F (diluted | | | | |
| | | | | | from 40 mg/mL | | | | |
| | | | | | formulation) | | | | |
| 6 | Compound | 8 | 8 | 1.6 | 5 mg/mL of | SC | 1 dose | 4 weeks | 672 |
| | A | | | | Formulation | | | | |
| | | | | | E/F (diluted | | | | |
| | | | | | from 40 mg/mL | | | | |
| | | | | | formulation) | | | | |
| 7 | Compound | 8 | 200 | 2 | 100 mg/ml of | IM | 1 dose | 4 weeks | 672 |
| | A | | | | Formulation G | | | | |
| 8 | Compound | 8 | 8 | 1.6 | 5 mg/mL of | IM | 1 dose | 4 weeks | 672 |
| | A | | | | Formulation | | | | |
| | | | | | EF1 (diluted | | | | |
| | | | | | from 40 mg/mL | | | | |
| | | | | | formulation) | | | | |

\hat{ }Vehicle for IV was 20% HP-β-CD in 5 mM citrate buffer of pH 3, with adjustment to a final PH of 5.
*IV formulation is 20% SBE-CD in 5 mM citrate buffer pH3 final pH to 5 adjusted by 1N NaOH

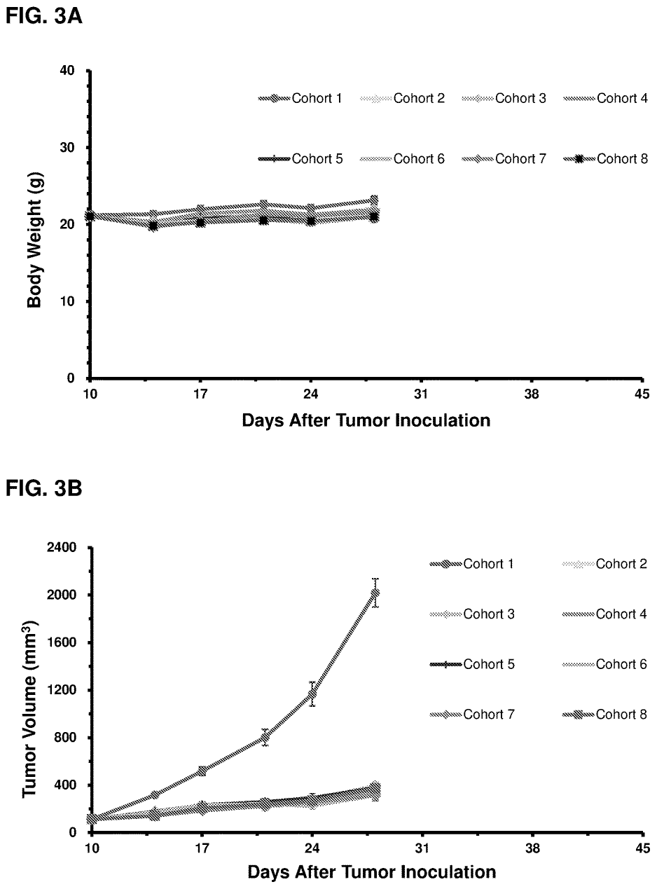

FIG. 3A shows body weight and FIG. 3B shows tumor volume results for the duration of the efficacy experiment. Animals tolerated all doses and all modes of administration similarly (FIG. 3A) with body weight remaining fairly constant throughout the experiment. Further, all doses and all modes of administration (Cohorts 2-8) slowed tumor growth significantly over 4 weeks in comparison to the Vehicle control (Cohort 1) (FIG. 3B). These data suggest that nanosuspension formulations of Compound A are a good format for long acting or sustained drug release, and that IV, SC, and IM modes of administration are effective.

### PK/PD:

Treatment (Table 9) starts when mean tumor volume reaches about 300-400 mm³ for the PKPD cohorts. Based on the tumor volume, mice are randomly assigned to respective groups using a computer-generated randomization procedure. Corresponding formulations are shown in Table 7. For each dosing cohort, dosing volume is calculated based on concentration of the formulation and dose volume (ml/kg). Vehicle for IV is 20% HP-β-CD in 5 mM citrate buffer of pH 3, with adjustment to a final PH of 5. Body weight of all animals is measured and recorded twice a week throughout the study. The measurement of tumor size is conducted twice a week with a caliper and recorded. Tumor volume is used for calculation of tumor growth inhibition (TGI) value of the tumors in the treated and the control cohorts, respectively, on a given day after tumor inoculation.

111 plasma samples are collected at five time points (4 h, 24 h, 96 h, 168 h and 336 h) from PK/PD cohorts 9-16 for PK analysis (n=3). 111 tumor samples are collected at five time points (4 h, 24 h, 96 h, 168 h and 336 h) post last dose from PK/PD cohorts 9-16 and cut into 3 pieces: one piece for PK analysis, one piece for WB analysis (BRD9), and one piece snap frozen for further analysis (n=3). Animals observed to be in a continuing deteriorating condition or their tumor size exceeding 2,000 mm³ will be euthanized prior to death, or before reaching a comatose state. An entire cohort will be terminated when the mean tumor exceeded 2000 mm³ in that cohort.

TABLE 9
Groups and Treatment for Mouse PKPD Experiment

| | | | | | Formulation & | | | | Collection |
|---|---|---|---|---|---|---|---|---|---|
| | | # Animals/ | Dose | Vol | Initial Conc. | | | Study | time points |
| Cohort | Drug | cohort | (mg/kg) | (ml/kg) | (mg/mL) | Route | Frequency | Duration | (hr) |
| 9 | Vehicle\hat{ } | 6 | — | 5 | Vehicle\hat{ } | IV | single dose | 4 weeks | 4, 24, 96, 168 and |
| | | | | | | | | | 336 |
| 10 | Compound | 15 | 1 | 5 | 0.2 mg/mL of | IV | single dose | 4 weeks | 4, 24, 96, 168 and |
| | A | | | | IV* | | | | 336 |
| 11 | Compound | 15 | 1 + 30 | 5 + 6 | 0.2 mg/mL of | IV + SC | single dose | 4 weeks | 4, 24, 96, 168 and |
| | A | | | | IV* + | | | | 336 |
| | | | | | 5 mg/mL of | | | | |
| | | | | | Formulation EF1 | | | | |
| | | | | | (diluted from 40 | | | | |
| | | | | | mg/mL | | | | |
| | | | | | formulation) | | | | |
| 12 | Compound | 15 | 200 | 5 | 40 mg/ml of | SC | single dose | 4 weeks | 4, 24, 96, 168 and |
| | A | | | | Formulation E/F | | | | 336 |
| 13 | Compound | 15 | 30 | 6 | 5 mg/mL of | SC | single dose | 4 weeks | 4, 24, 96, 168 and |
| | A | | | | Formulation EF1 | | | | 336 |
| | | | | | (diluted from 40 | | | | |
| | | | | | mg/mL | | | | |
| | | | | | formulation) | | | | |
| 14 | Compound | 15 | 8 | 1.6 | 5 mg/mL of | SC | single dose | 4 weeks | 4, 24, 96, 168 and |
| | A | | | | Formulation EF1 | | | | 336 |
| | | | | | (diluted from 40 | | | | |
| | | | | | mg/mL | | | | |
| | | | | | formulation) | | | | |
| 15 | Compound | 15 | 200 | 2 | 100 mg/ml of | IM | single dose | 4 weeks | 4, 24, 96, 168 and |
| | A | | | | Formulation G | | | | 336 |
| 16 | Compound | 15 | 8 | 1.6 | 5 mg/mL of | IM | single dose | −4 weeks | 4, 24, 96, 168 and |
| | A | | | | Formulation EF1 | | | | 336 |
| | | | | | (diluted from 40 | | | | |
| | | | | | mg/mL | | | | |
| | | | | | formulation) | | | | |

\hat{ }Vehicle for IV was 20% HP-β-CD in 5 mM citrate buffer of pH 3, with adjustment to a final PH of 5.
*IV formulation is 20% SBE-CD in 5 mM citrate buffer pH3 final pH to 5 adjusted by 1N NaOH

### Example 2: Amorphous Microsuspension Formulations of Compound A

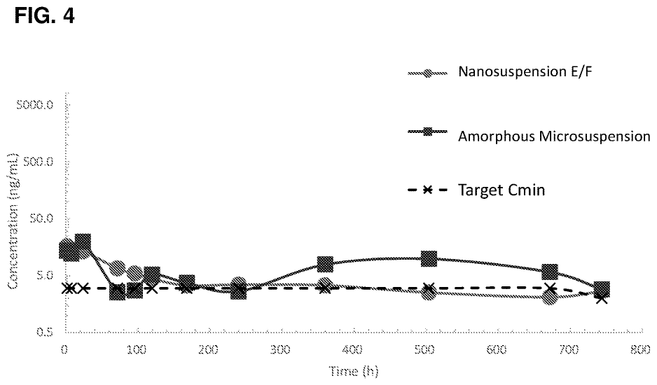

Compound A in a concentration range of between about 20 mg/mL to about 100 mg/mL (2-10% w/w), along with parenteral grade olive oil and 1% sorbitan monooleate [SPAN®80], was nano-milled using a high energy mill at 3000 rpm with 0.3 mm beads or 0.65 mm beads to produce microsuspensions. According to X-ray powder diffraction (XRPD), the microsuspensions were confirmed to be amorphous. PSD of the microsuspension formulations ranged from a D50 of about 3 microns to about 0.7 microns. Performance of an amorphous microsuspension of Compound A was evaluated in Male SD rats. The animals were approximately 6-8 weeks of age and approximately 200-300 g at time of dosing. Each of the amorphous microsuspension and amorphous microsuspension formulations were dosed at 20 mg/kg (Table 8) and administered subcutaneously. At each time point, approximately 0.2 mL blood was collected via jugular vein and analyzed by LC-MS/MS for mean plasma concentration of Compound A (FIG. 4). Pharmacokinetics determined that a target Cmin of 3 ng/mL was desired, which is denoted by the black dashed line in FIG. 4.

This pharmacokinetic study in rats (n=3) at 20 mg/kg with an amorphous microsuspension showed that this formulation was able to meet the criteria for target Cmin of 3 ng/mL for at least 4 weeks. The nanosuspension formulation, dosed at 20 mg/kg met the target Cmin for at least 2 weeks. (FIG. 4). In comparison, a solution formulation administered by IV infusion in rats up to 8 mg/kg did not meet target Cmin for two weeks. Also, as another point of comparison, a suspension of “as-is” drug substance in normal saline when administered via sub-cutaneous route in SD rats was not able to provide a sustained/extended release and the plasma concentration was <target Cmin at all the time points.

TABLE 10
Rat PK Study Parameters of Amorphous
Microsuspension Formulation

| | | Initial | Dose | Route |
|---|---|---|---|---|
| | | concentration | admin- | of |
| | | of Formulation | istered | admin- |
| Cohort | Formulation | (mg/mL) | (mg/kg) | istration |
| 1 | Parenteral grade | 20 | 20 | SC |
| | olive oil and 1% | | | |
| | sorbitan | | | |
| | monooleate | | | |
| | [SPAN ®80], | | | |
| 2 | Nanosuspension | 40 | 20 | SC |
| | E/F | | | |

### Example 3: PLGA Microsphere and PLGA In-Situ Gel Formulations of Compound a

### PLGA Microspheres Preparation

Two different types of PLGA, RG503 and RG503H, were utilized for microencapsulation of Compound A. Both types of PLGA had a ratio of 50% lactic acid to 50% glycolic acid. RG503 has an ester N-terminus, and RG503H has an acid N-terminus. Dichloromethane (DCM) was used to dissolve both Compound A and PLGA into a solution. The DCM solution was then slowly added dropwise into an aqueous excipient solution, herein referred to as an aqueous surfactant solution. Three different surfactants, poly(vinyl alcohol) (PVA), POLOXAMER®188 (P-188), and POLOXAMER®407 (P-407) were screened at various concentrations and temperatures. The DCM solution to aqueous surfactant solution ratio was also varied in the screen. The aqueous surfactant solutions were stirred with a high-speed homogenizer throughout the addition of the DCM solution. Following DCM solution addition, the emulsion was stirred to allow the DCM to evaporate. PLGA microspheres were isolated by centrifugation and washed with water. Final drying was conducted under vacuum at room temperature.

### In Situ PLGA Gel Preparation

In situ PLGA gel formulations were prepared (see Table 11) by weighing a desired amount of Compound into a solvent-compatible container. A solvent was added to the container and vortexed and/or sonicated to create a solution. A polymer was added to the container, and moderate agitation was applied until a clear solution was obtained. The composition of the formulations is shown in Table 11. As shown, PLGA and the solvent system were varied.

TABLE 11
In situ Gel Formulation Composition

| | Com- | | PLGA | | Solvent |
|---|---|---|---|---|---|
| | pound | | Concen- | | Concen- |
| Batch | A | | tration | | tration |
| Number | (wt %) | PLGA | (wt %) | Solvent¹ | (wt %) |
| Gel 1 | 8 | RG502 | 20 | DMA | 72 |
| Gel 2 | 10 | RG502 | 20 | 50:50 | 70 |
| | | | | DMSO:NMP | |
| Gel 3 | 10 | RG502 | 20 | DMF | 70 |
| Gel 4 | 20 | RG502 | 20 | 50:50 | 60 |
| | | | | DMA:NMP | |
| Gel 5 | 20 | RG502 | 30 | 50:50 | 50 |
| | | | | DMA:NMP | |
| Gel 6 | 20 | RG502H | 20 | 50:50 | 60 |
| | | | | DMA:NMP | |
| Gel 7 | 8 | RG502H | 20 | DMA | 72 |
| Gel 8 | 10 | RG502H | 30 | DMF | 60 |

DMA = Dimethylacetamide,
DMSO = Dimethyl sulfoxide,
NMP = N-methyl-2-pryrrolidone,
DMF = Dimethylformamide

In situ PLGA Gel Injection Force Testing

Injectability of in situ PLGA gels was determined using 23 g, 1 inch, thin-wall needles. Representative formulations comprised of 20% RG502H in DMF and 30% RG502H in DMF, respectively, were evaluated with an Instron 6800 equipped with a 100 N load cell. All formulations were readily injectable. The injection force was 3.1 Newton for the lower PLGA concentration and 7.3 Newton for the higher PLGA concentration.

### Example 4: SAIB Nanosuspension Formulations of Compound A

A sucrose acetate isobutyrate (SAIB) and DMSO solution was prepared by transferring 4 mL of DMSO to a glass vial. SAIB was heated to 60-80° C. in an oven and/or hot plate and liquified SAIB was added to the vial containing the DMSO. The vial was placed on a roller mill until a homogenous solution was achieved. Compound A was added to the ball mill chamber and milled at a specified speed and duration (Table 12). The SAIB solution was transferred to the ball mill chamber and milling media beads were added to the chamber. The drug particles were milled until a nanosuspension was obtained.

In another example, a SAIB nanosuspension was prepared using the same method provided above with the incorporation of 0.5% Tween 20 as surfactant.

TABLE 12
SAIB Nanosuspension Formulations

| Batch | Batch Size | Mill | Milling | Particle |
|---|---|---|---|---|
| Number | (mL) | Speed (hz) | Time (hr) | Size, D(50) |
| 1 | 1 | 40 | 6 | 0.391 micron |
| 2 | 4 | 40 | 2 | 0.255 micron |

Injectability of an SAIB nanosuspension was determined using 23 g, 1 inch, thin-wall needles. A 10% Compound A, 65% SAIB/0.5% Tween 20, 24.5% DMSO formulation was evaluated with an Instron 6800 equipped with a 100 N load cell. The injection force was 10.87 N and the formulation was readily injectable. In summary, SAIB nanosuspension formulations were successfully prepared with compound A and represents a viable long acting formulation option for dosing Compound A.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A pharmaceutically acceptable composition comprising a long-acting formulation of a compound that reduces the level and/or activity of a protein, or a pharmaceutically acceptable salt thereof, and a pharmaceutically acceptable excipient.

2. A pharmaceutically acceptable composition comprising a long-acting formulation of a compound having the structure of Formula I: 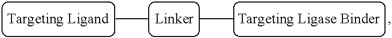
wherein, the Targeting Ligand is a group that is capable of binding to a target protein; the Linker is a group that covalently links the Targeting Ligand to the Targeting Ligase Binder;
the Targeting Ligase Binder is a group that is capable of binding to a ligase;
or a pharmaceutically acceptable salt thereof, and a pharmaceutically acceptable excipient.

3. The compound of claim 2, wherein the targeting ligase binder is an E3 ligase ligand.

4. The compound of claim 2, wherein the targeting ligase binder is an E3 ubiquitin ligase (cereblon) binding moiety, VHL E3 ubiquitin ligase binding moiety, MDM2 (i.e. human double minute 2 or HDM2) E3 ligase binding moiety, or IAP E3 ubiquitin ligase binding moiety.

5. The compound of claim 2, wherein the targeting ligase binder comprises the structure of Formula A: 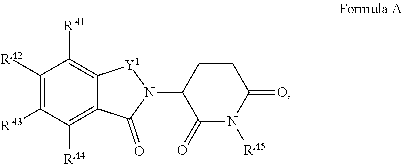
wherein
each of R^{A1}, R^{A2}, R^{A3}, and R^{A4} is, independently, H, a bond to the linker, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form optionally substituted C₆-C₁₀ aryl, optionally substituted C₃-C₁₀ carbocyclyl, optionally substituted C₂-C₉ heteroaryl, or C₂-C₉ heterocyclyl;
R^{A5} is H, optionally substituted C₁-C₆ alkyl, or optionally substituted C₁-C₆ heteroalkyl;
Y¹ is 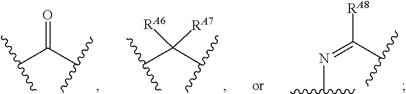
each of R^{A6} and R^{A7} is, independently, H or optionally substituted C₁-C₆ alkyl; or R^{A6} and R^{A7}, together with the carbon atom to which each is bound, form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
where one of R^{A1}, R^{A2}, R^{A3}, and R^{A4} is a bond to the linker,
or a pharmaceutically acceptable salt thereof.

6. The compound of claim 5, wherein each R^{A1}, R^{A2}, R^{A3}, and R^{A4} is, independently, a bond to the linker, H, optionally substituted C₁-C₆ alkyl, optionally substituted C₁-C₆ heteroalkyl, hydroxyl, optionally substituted amino; or R^{A1} and R^{A2}, R^{A2} and R^{A3}, or R^{A3} and R^{A4}, together with the carbon atoms to which each is attached, combine to form optionally substituted C₂-C₉ heterocyclyl.

7. The compound of claim 5, wherein the structure of Formula A is: 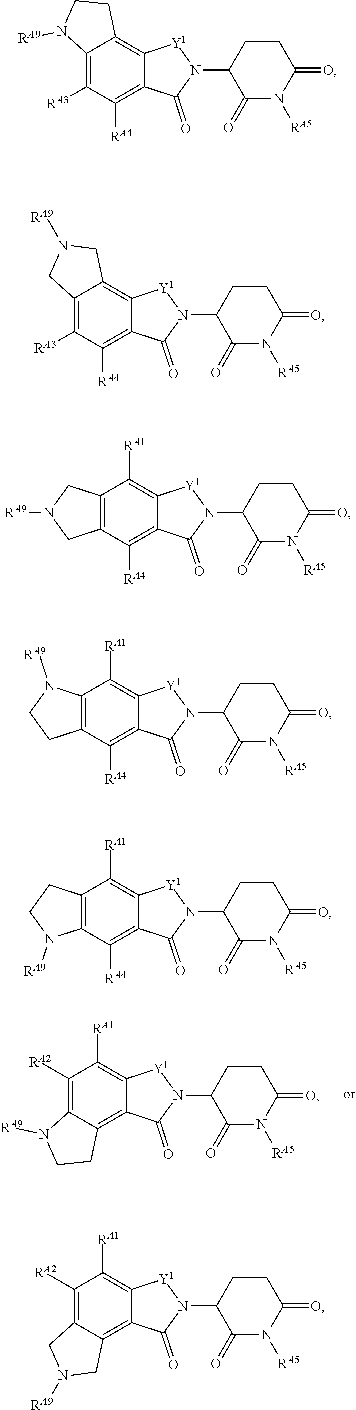
wherein R^{A9} is H, optionally substituted C₁-C₆ alkyl, or optionally substituted C₁-C₆ heteroalkyl.

8. The compound of any one of claims 5 to 7, wherein R^{A5} is H or optionally substituted C₁-C₆ alkyl.

9. The compound of claim 8, wherein R^{A5} is H or methyl.

10. The compound of claim 9, wherein R^{A5} is H.

11. The compound of claim 10, wherein R^{A5} is methyl.

12. The compound of any one of claims 5 to 11, wherein Y¹ is

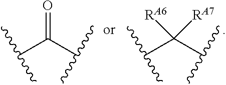

13. The compound of claim 12, wherein Y¹ is

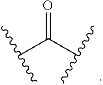 [SMILES: *C(*)=O]

14. The compound of claim 12, wherein Y¹ is

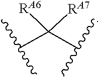

15. The compound of claim 14, wherein Y¹ is

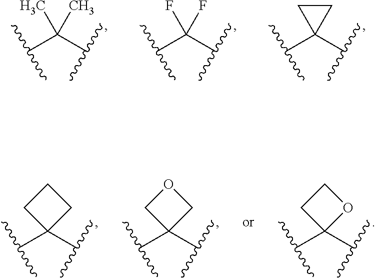

16. The compound of any one of claims 5 to 15, wherein the structure of Formula A has the structure of Formula A1: 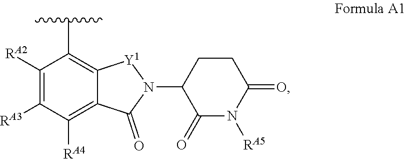
or a pharmaceutically acceptable salt thereof.

17. The compound of any one of claims 5 to 15, wherein the structure of Formula A has the structure of Formula A2: 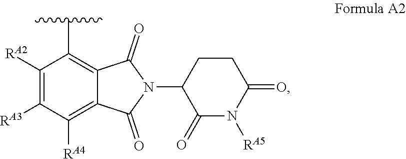
or a pharmaceutically acceptable salt thereof.

18. The compound of any one of claims 5 to 15, wherein the structure of Formula A has the structure of Formula A3: 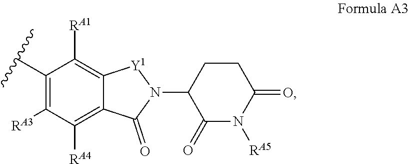
or a pharmaceutically acceptable salt thereof.

19. The compound of any one of claims 5 to 18, wherein the structure of Formula A is 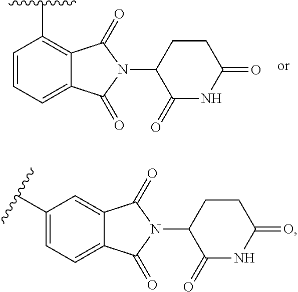
or is a derivative or an analog thereof.

20. The compound of any one of claims 1 to 4, wherein the targeting ligase binder comprises the structure of Formula B: 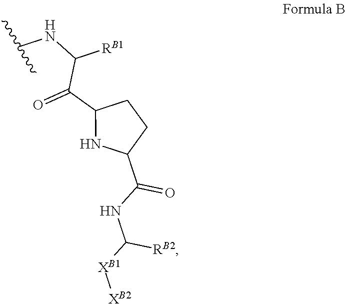
where
each of R^{B1}, and R^{B2} is, independently, H, a bond to the linker, halogen, optionally substituted C₁-C₆ alkyl, optionally substituted C₁-C₆ heteroalkyl, or optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with CH₃, OH, OCH₃ and/or F.
each X^{B1} is a bond to the linker, halogen, optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with one or more groups of R^{B3};
where R^{B3} is optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl,
each X^{B2} is a bond to the linker, halogen, —CN, optionally substituted C₁-C₆ alkyl, optionally substituted C₁-C₆ heteroalkyl, optionally substituted C₃-C₁₀ carbocyclyl, wherein each C₁₋₆ alkyl, optionally substituted C₁-C₆ heteroalkyl, or optionally substituted C₃-C₁₀ carbocyclyl, is optionally substituted with one or more groups of R^{B4}; where R^{B4} is optionally substituted C₁-C₆ alkyl, optionally substituted C₁-C₆ heteroalkyl, optionally substituted C₃-C₁₀ carbocyclyl, wherein each C₁-C₆ alkyl, optionally substituted C₁-C₆ heteroalkyl, or optionally substituted C₃-C₁₀ carbocyclyl, is optionally substituted with one or more groups of R^{B5};
where R^{B5} is CH₃, OH, OCH₃ and/or F.

21. The compound of claim 20, wherein R^{B1} is

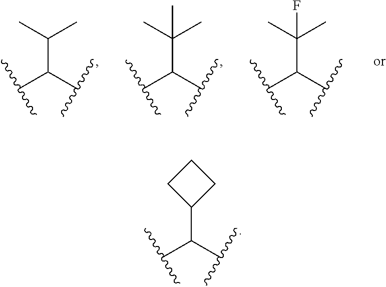

22. The compound of claim 20, wherein R^{B2} is CH₃ CH₂OH, or CH₂F.

23. The compound of claim 20, wherein X^{B1} is

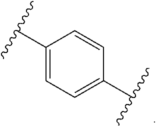 [SMILES: *c1ccc(*)cc1]

24. The compound of claim 20, wherein X^{B2} is

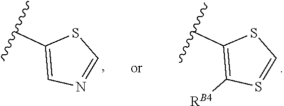

25. The compound of claim 20, wherein X^{B2} is CN.

26. The compound of claim 20, wherein X^{B2} is

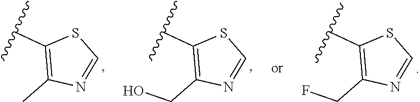

27. The compound of claim 2, wherein the targeting ligase binder has the structure:

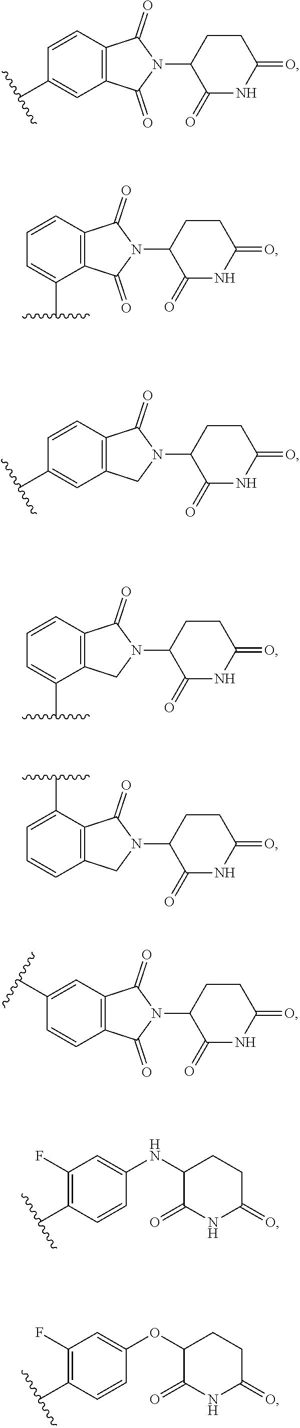

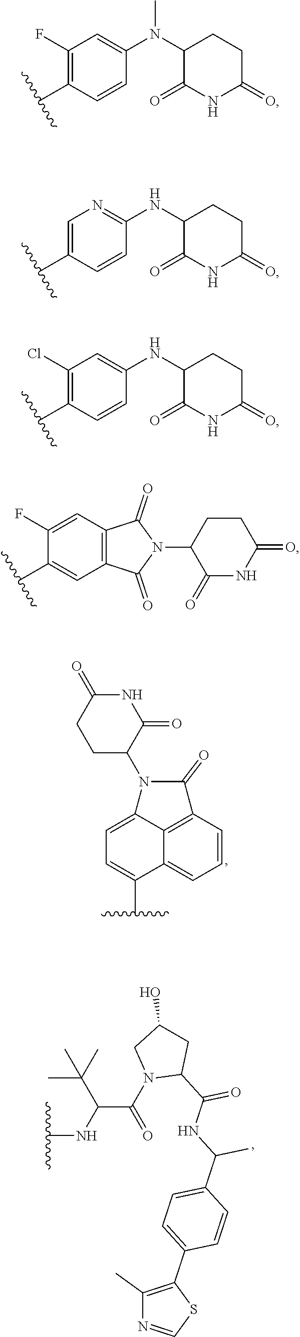 [SMILES: *c1ccc(N(C)C2CCC(=O)NC2=O)cc1F ; *c1ccc(NC2CCC(=O)NC2=O)nc1 ; *c1ccc(NC2CCC(=O)NC2=O)cc1Cl ; *c1cc2c(cc1F)C(=O)N(C1CCC(=O)NC1=O)C2=O ; *c1ccc2c3c(cccc13)C(=O)N2C1CCC(=O)NC1=O ; *NC(C(=O)N1C[C@H](O)CC1C(=O)NC(C)c1ccc(-c2scnc2C)cc1)C(C)(C)C]

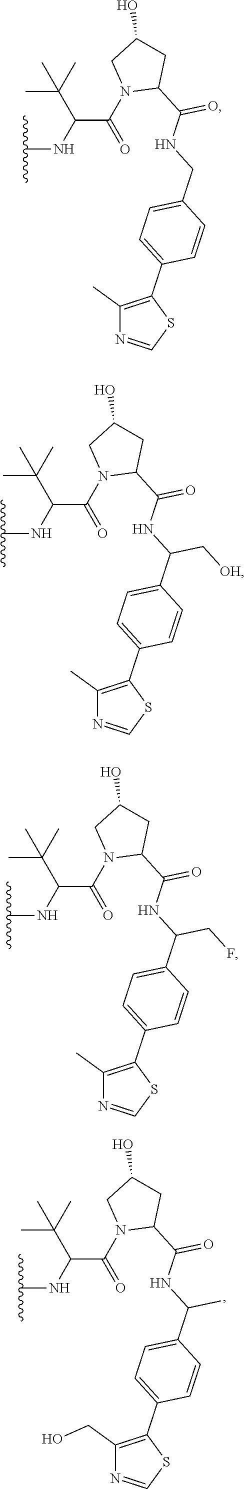

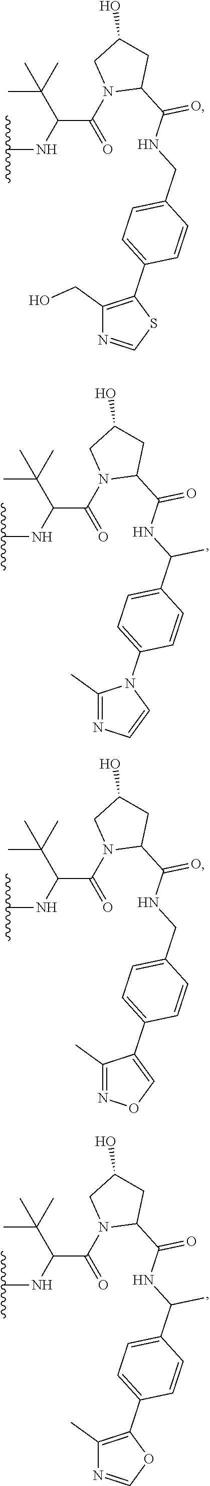

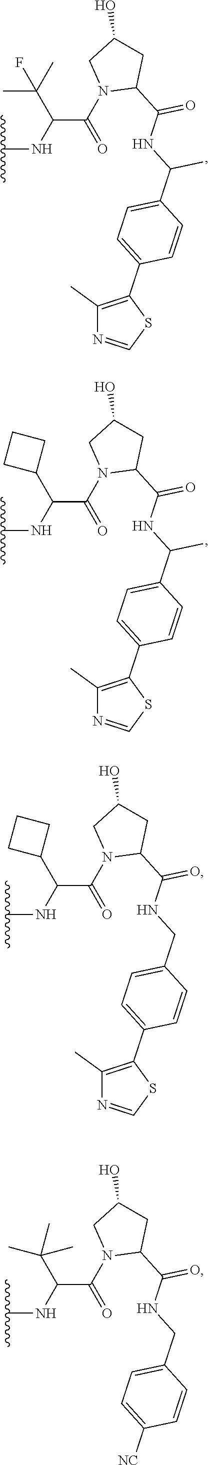

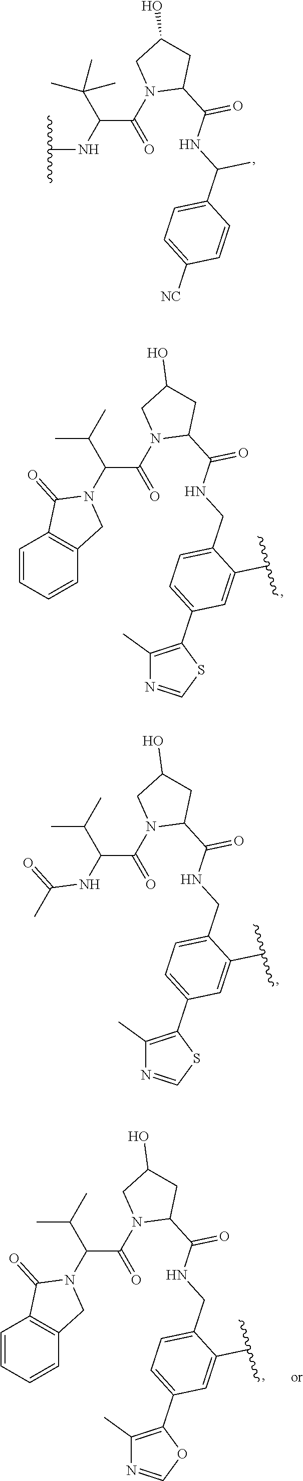 [SMILES: *NC(C(=O)N1C[C@H](O)CC1C(=O)NC(C)c1ccc(C#N)cc1)C(C)(C)C ; *c1cc(-c2scnc2C)ccc1CNC(=O)C1CC(O)CN1C(=O)C(C(C)C)N1Cc2ccccc2C1=O ; *c1cc(-c2scnc2C)ccc1CNC(=O)C1CC(O)CN1C(=O)C(NC(C)=O)C(C)C ; *c1cc(-c2ocnc2C)ccc1CNC(=O)C1CC(O)CN1C(=O)C(C(C)C)N1Cc2ccccc2C1=O]

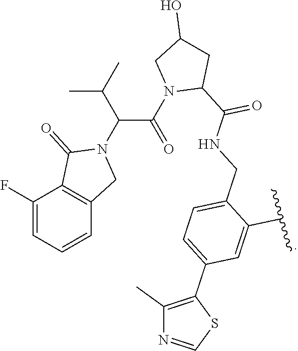 [SMILES: *c1cc(-c2scnc2C)ccc1CNC(=O)C1CC(O)CN1C(=O)C(C(C)C)N1Cc2cccc(F)c2C1=O]

28. The compound of claim 2, wherein the targeting ligase binder has the structure: 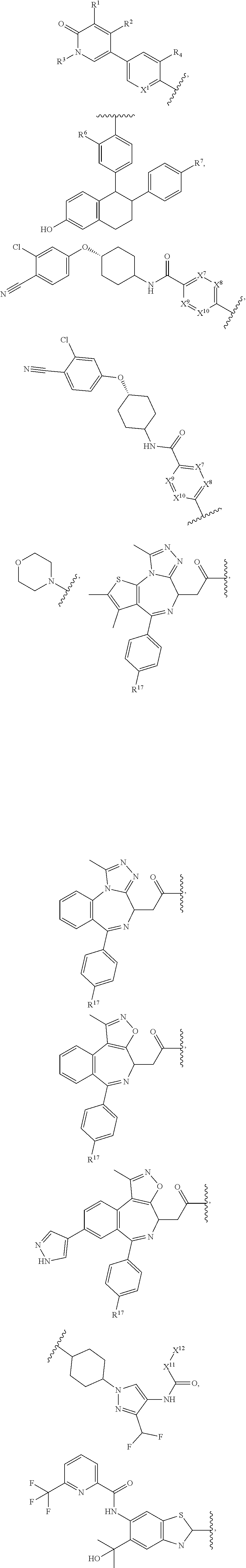 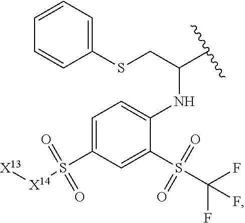
R¹ is NH₂, or CH₃;
R² is H, or CH₃;
R³ is H, CH₃ or OCH₃;
R⁴ is H, CH₃, OCH₃, CF₂H, or CF₃; R⁶ is H, F or CH₃,
R⁷ is H, CH₃, OCH₃, or OH;
X¹ is OH, N, COCH₃, or CCF₃;
X⁷ is CH₂ or N;
X⁸ is CH₂ or N;
X⁹ is CH₂ or N;
X¹⁰ is CH₂ or N; X¹¹ is

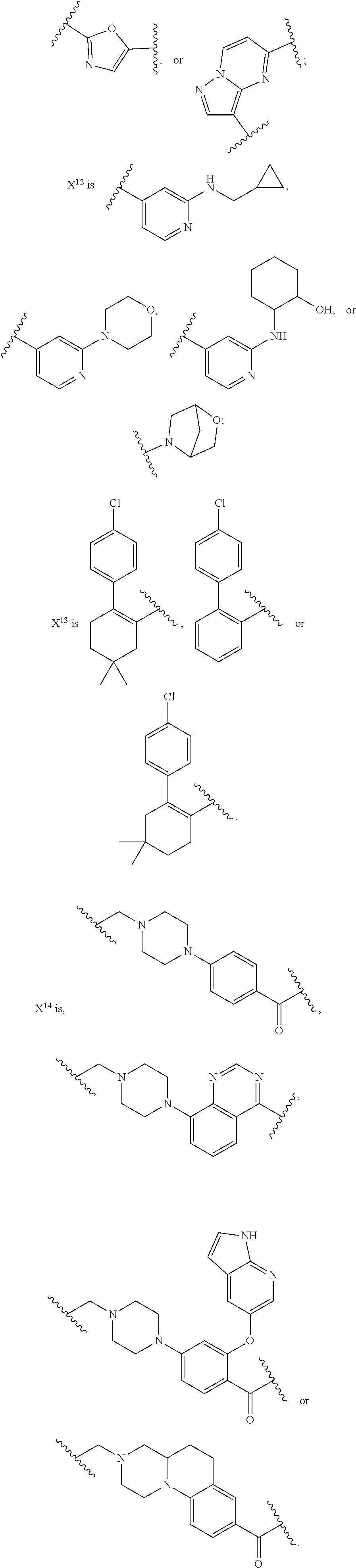

29. The compound of claim 2, wherein the targeting ligase binder has the structure:

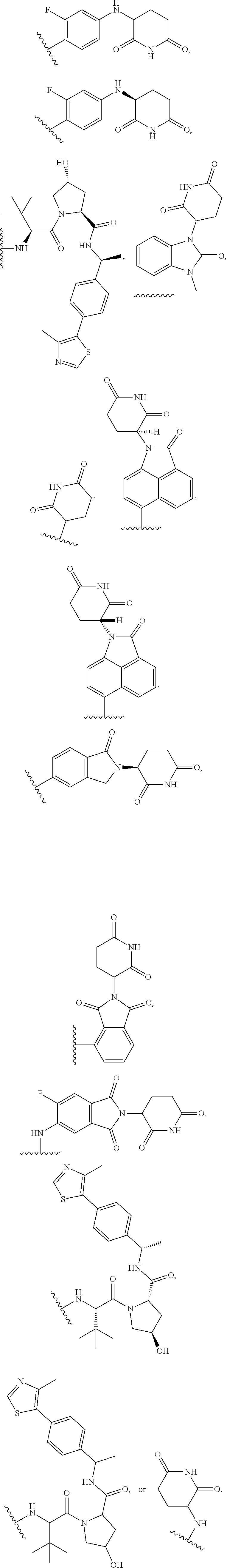

30. The compound of claim 2, wherein the targeting ligase binder comprises the structure of Formula IVa: 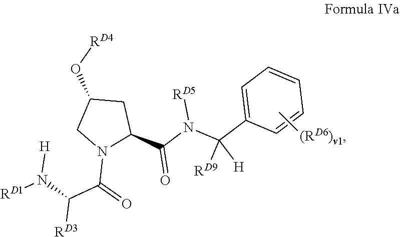
wherein
R^{D1} is H, A², —C(O)-A², optionally substituted C₁-C₆ alkyl, or optionally substituted C₁-C₆ heteroalkyl;
R^{D3} is A², —C(O)-A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl, optionally substituted C₃-C₁₀ carbocyclyl, or optionally substituted C₁-C₆ alkyl wherein the C₁-C₆ alkyl, C₁-C₆ heteroalkyl, C₃-C₁₀ carbocyclyl, C₆-C₁₀ aryl, C₁-C₆ alkyl, C₃-C₁₀ carbocyclyl, or C₆-C₁₀ aryl is substituted with A² and/or one of more groups of R^{J2};
R^{D4} is H, optionally substituted C₁-C₆ alkyl, optionally substituted C₃-C₁₀ carbocyclyl, or optionally substituted C₆₋₁₀ aryl;
R^{D5} is H, optionally substituted C₁-C₆ alkyl, or optionally substituted C₁-C₆ heteroalkyl;
v1 is 0, 1, 2, 3, or 4;
each R^{D6} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted C₂-C₆ alkynyl, hydroxy, thiol,
R^{D9} is H, or optionally substituted C₁-C₆ alkyl;
each R^{J2} is, independently, hydrogen, C₁-C₆ alkyl, C₃-C₁₂ carbocyclyl, and C₃-C₁₂ heterocyclyl, wherein each C₁-C₆ alkyl, C₃-C₁₂ carbocyclyl, and C₃-C₁₂ heterocyclyl is optionally substituted with one or more groups independently selected from amino, hydroxyl, C₁-C₆ alkoxy, C₃-C₁₂ carbocyclyl, C₃-C₁₂ heterocyclyl, or C₁-C₆ alkyl that is optionally substituted with A² and/or one or more groups independently selected from oxo and halo; and
wherein either R^{D1} or R^{D3} comprises A² or —C(O)-A²;
wherein A² is a bond to the linker;
or a pharmaceutically acceptable salt thereof.

31. The compound of claim 30, wherein R^{D6} is optionally substituted C₂-C₉ heteroaryl.

32. The compound of any one of claims 30-31, wherein R^{D6} is:

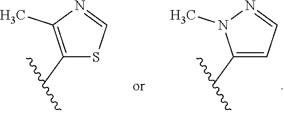

33. The compound of any one of claims 30-32, wherein the structure of Formula IVa is 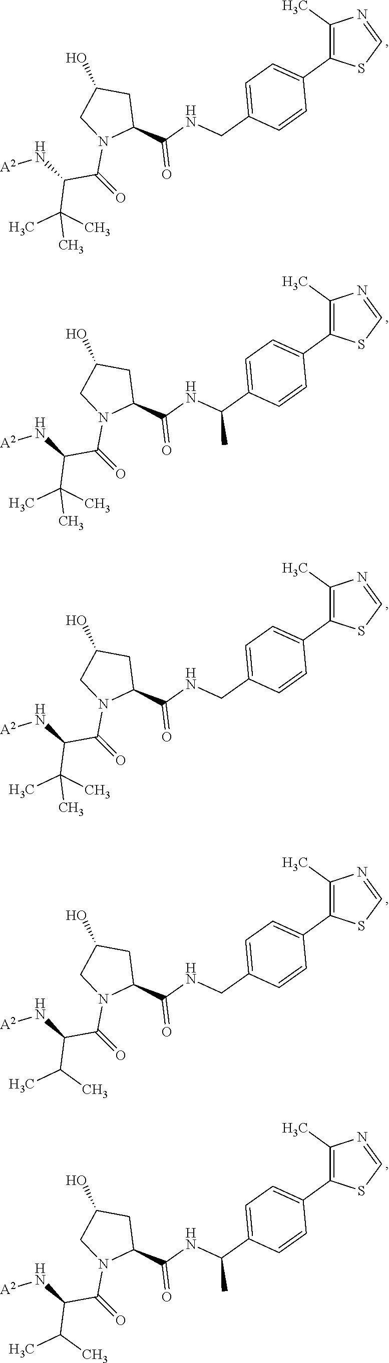 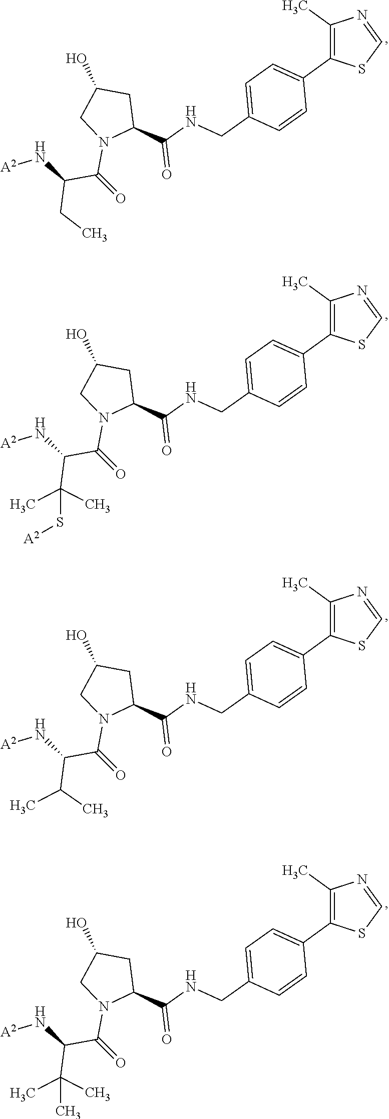
or a derivative or analog thereof.

34. The compound of claim 30, wherein R^{D6} is halogen or optionally substituted C₂-C₆ alkynyl.

35. The compound of claim 30, wherein R^{D6} is optionally substituted C₁-C₆ heteroalkyl.

36. The compound of claim 35, wherein optionally substituted C₁-C₆ heteroalkyl is methoxy.

37. The compound of claim 35 or 36, wherein the structure of Formula IVa is 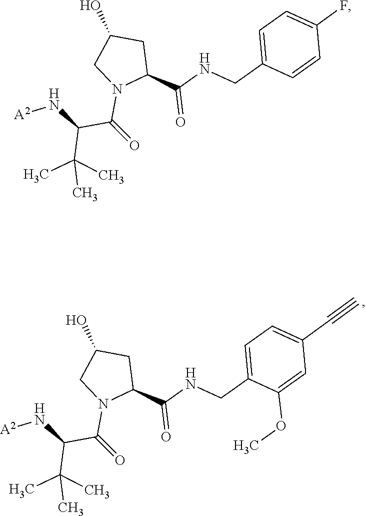
or a derivative or analog thereof.

38. The compound of claim 2, wherein the targeting ligase binder comprises the structure of Formula IV: 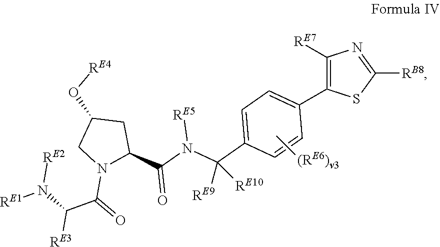
wherein
R^{E1} is H, A², optionally substituted C₁-C₆ alkyl, or optionally substituted C₁-C₆ heteroalkyl;
R^{E2} is H, optionally substituted C₁-C₆ alkyl, or optionally substituted C₁-C₆ heteroalkyl;
R^{E3} is A², optionally substituted C₁-C₆ alkyl, optionally substituted C₁-C₆ heteroalkyl, optionally substituted C₃-C₁₀ carbocyclyl, or optionally substituted C₆-C₁₀ aryl;
R^{E4} is H, optionally substituted C₁-C₆ alkyl, optionally substituted C₃-C₁₀ carbocyclyl, or optionally substituted C₆-C₁₀ aryl;
R^{E5} is H, optionally substituted C₁-C₆ alkyl, or optionally substituted C₁-C₆ heteroalkyl;
v3 is 0, 1, 2, 3, or 4;
each R^{BE} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R^{E7} and R^{E8} is, independently, H, halogen, optionally substituted C₁-C₆ alkyl, or optionally substituted C₆-C₁₀ aryl,
R^{E9} and R^{E10} are, independently, H, or optionally substituted C₁-C₆ alkyl,
wherein one of R^{E1} and R^{E3} is A²;
wherein A² is a bond to the linker;
or a pharmaceutically acceptable salt thereof.

39. The compound of claim 38, wherein the structure of Formula IV is 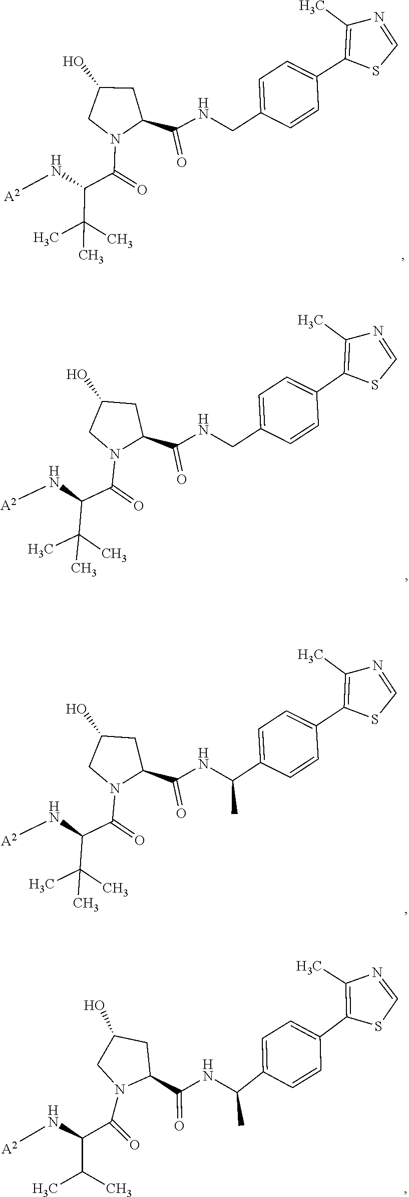 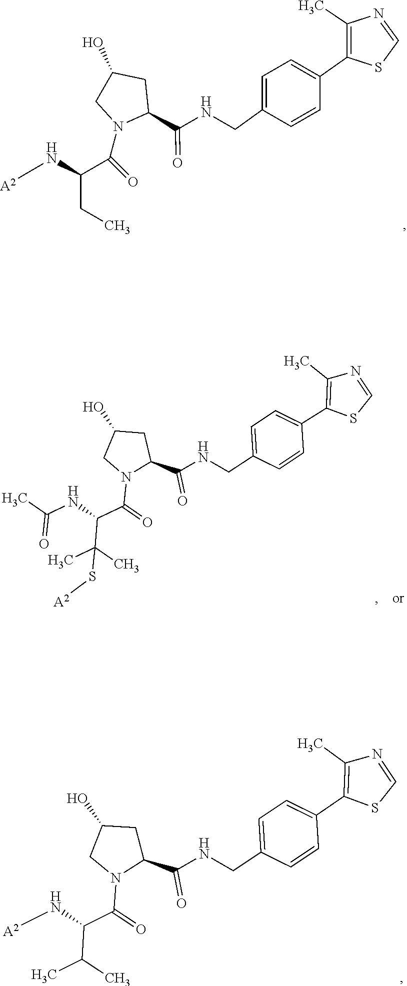
or derivative or analog thereof.

40. The compound of claim 39, wherein the targeting ligase binder has the structure:

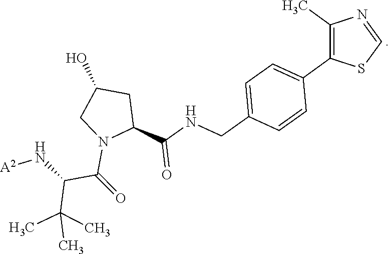

41. The compound of claim 39, wherein the targeting ligase binder has the structure:

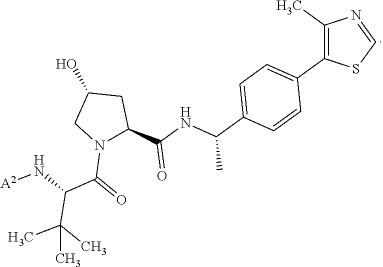

42. The compound of claim 2, wherein the targeting ligase binder comprises the structure of Formula V: 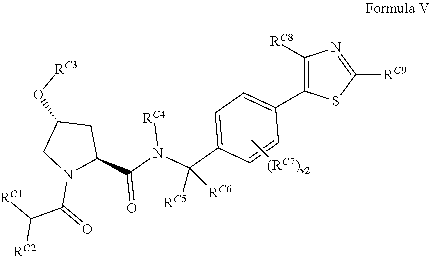
R^{C1} is optionally substituted C₁-C₆ alkyl;
R^{C2} is A², or optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₂-C₉ heteroaryl, wherein each C₁-C₆ alkyl, C₃-C₁₀ carbocyclyl, C₁-C₆ heteroalkyl, C₆-C₁₀ aryl, or C₂-C₉ heteroaryl is optionally substituted with A² and/or one or more groups R^{J};
R^{C3} is H, optionally substituted C₁-C₆ alkyl, optionally substituted C₃-C₁₀ carbocyclyl, or optionally substituted C₆-C₁₀ aryl;
R^{C4} is H, optionally substituted C₁-C₆ alkyl, or optionally substituted C₁-C₆ heteroalkyl;
v2 is 0, 1, 2, 3, or 4;
each of R^{C5} and R^{C6} is, independently, H, or optionally substituted C₁-C₆ alkyl
each R^{C7} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R^{C8} and R^{C9} is, independently, H, halogen, optionally substituted C₁-C₆ alkyl, or optionally substituted C₆-C₁₀ aryl,
each R^{J} is, independently, hydrogen, C₁-C₆ alkyl, C₃-C₆ carbocyclyl, and C₂-C₆ heterocyclyl, wherein each C₁-C₆ alkyl, C₃-C₆ carbocyclyl, and C₂-C₆ heterocyclyl is optionally substituted with one or more groups independently selected from amino, hydroxyl, C₁-C₆ alkoxy, C₃-C₆ carbocyclyl, C₂-C₆ heterocyclyl, or C₁-C₆ alkyl that is optionally substituted with A² and/or one or more groups independently selected from oxo and halo;
wherein A² is a bond to the linker;
or a pharmaceutically acceptable salt thereof.

43. The compound of claim 42, wherein the targeting ligase binder has the structure:

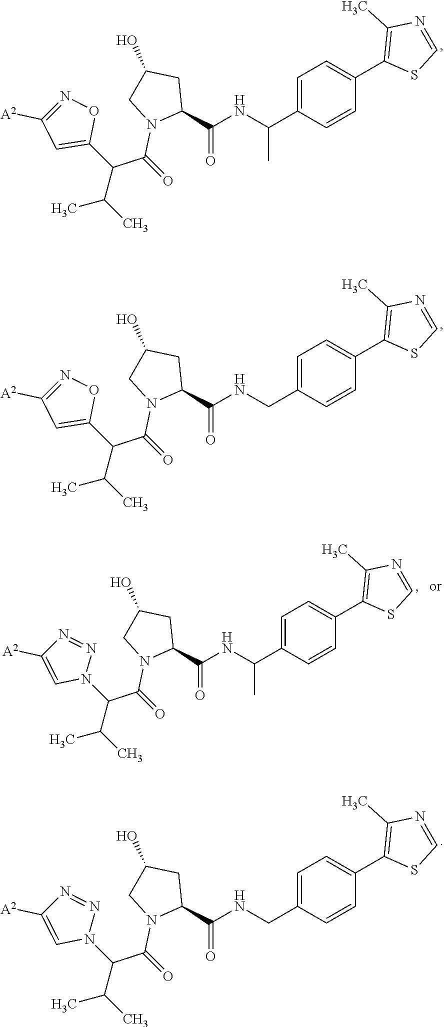

44. The compound of claim 2, wherein the targeting ligase binder comprises the structure of Formula IVb: 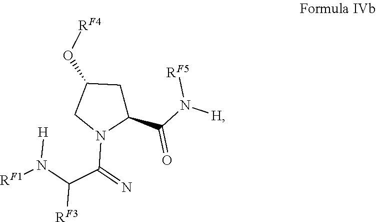
wherein
R^{F1} is H, A², —C(O)-A², optionally substituted C₁-C₆ alkyl, or optionally substituted C₁-C₆ heteroalkyl;
R^{F3} is A², —C(O)-A², optionally substituted C₁-C₆ alkyl, optionally substituted C₁-C₆ heteroalkyl, optionally substituted C₃-C₁₀ carbocyclyl, or optionally substituted C₆-C₁₀ aryl;
R^{F4} is H, optionally substituted C₁-C₆ alkyl, optionally substituted C₃-C₁₀ carbocyclyl, optionally substituted C₆₋₁₀ aryl;
R^{F5} is H, optionally substituted C₁-C₆ alkyl, or optionally substituted C₁-C₆ heteroalkyl or optionally substituted arylalkyl;
wherein one of R^{F1} and R^{F3} is A² or —C(O)-A²;
wherein A² is a bond to the linker;
or a pharmaceutically acceptable salt thereof.

45. The compound of claim 44, wherein the structure of Formula IVb is 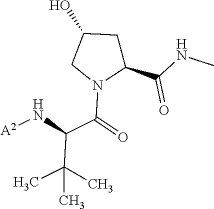
or a derivative or analog thereof.

46. The compound of claim 2, wherein the targeting ligand has the structure:

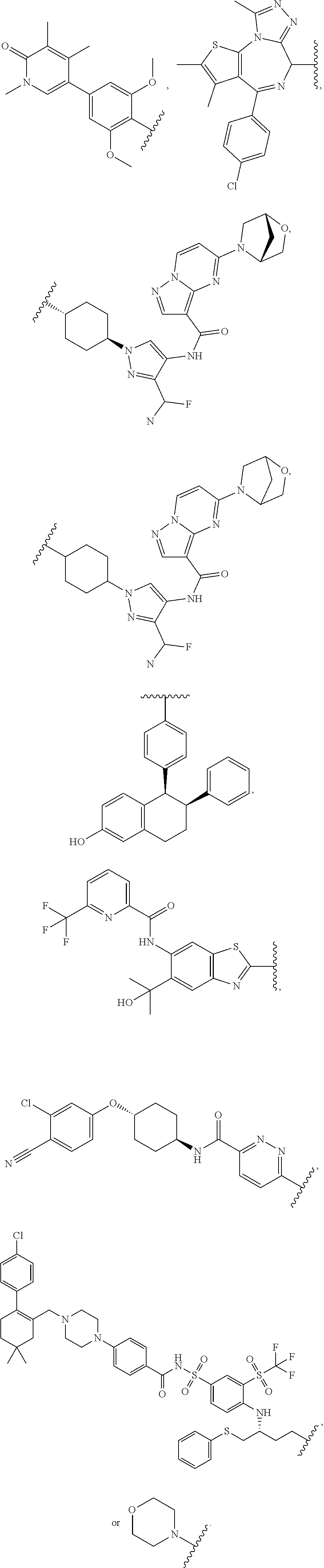

47. The compound of claim 2, wherein the targeting ligand has the structure:

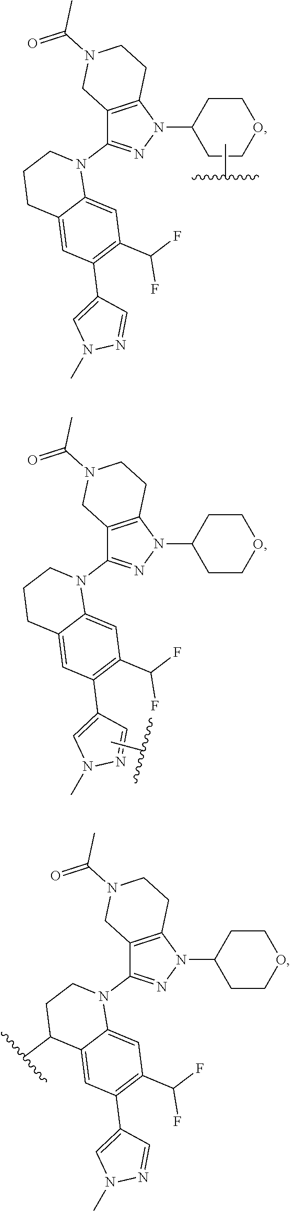

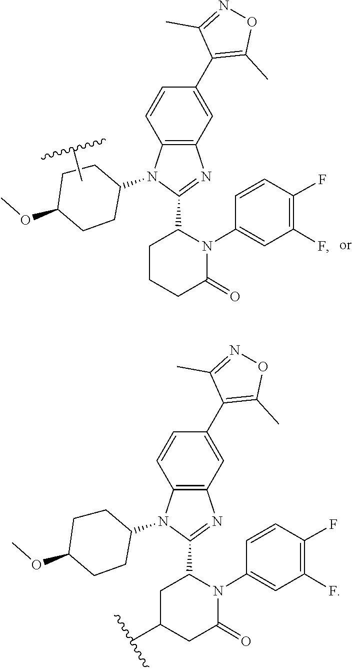

48. The compound of claim 2, wherein the linker has the structure: 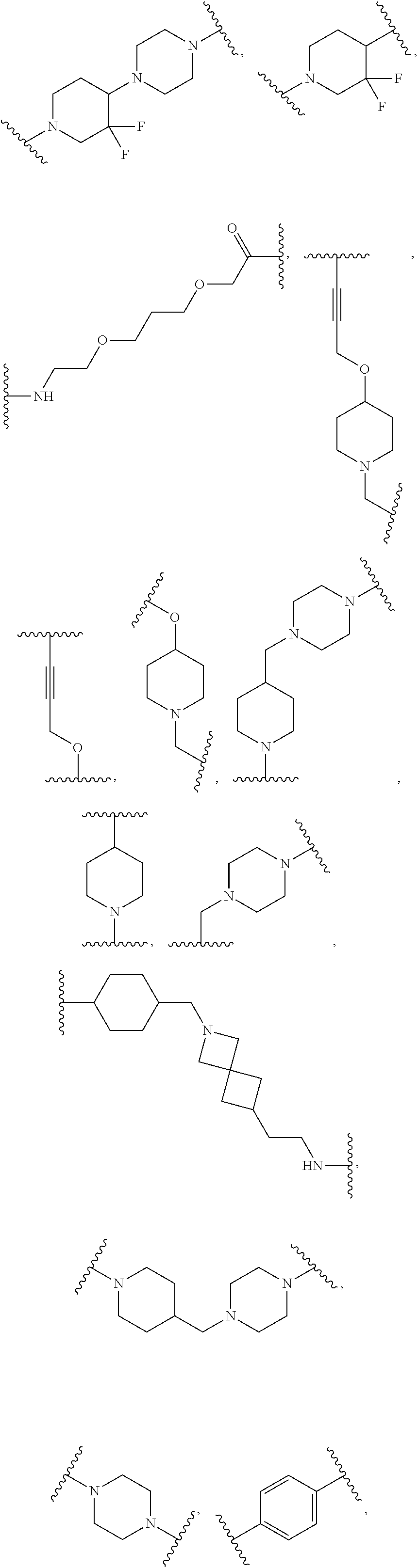
—N(R)—(CH2)ₘ-O(CH2)ₙ-O(CH2)ₒ-O(CH2)ₚ-O(CH2)_{q}-O(CH2)ᵣOCH2-, —O—(CH2)ₘ-O(CH2)ₙ-O(CH2)ₒ-O(CH2)ₚ-O(CH2)_{q}-O(CH2)ᵣ-OCH2-, —O—(CH2)ₘ-O(CH2)ₙ-O(CH2)ₒ-O(CH2)ₚ-O(CH2)_{q}-O(CH2)ᵣ-O—, —N(R)—(CH2)ₘ-O(CH2)ₙ-O(CH2)ₒ-O(CH2)ₚ-O(CH2)_{q}-O(CH2)ᵣ-O—, —(CH2)ₘ-O(CH2)ₙ-O(CH2)ₒ-O(CH2)ₚ-O(CH2)_{q}-O(CH2)ᵣ-O—, —(CH2)ₘ-O(CH2)ₙ-O(CH2)ₒ-O(CH2)ₚ-O(CH2)_{q}-O(CH2)ᵣ-OCH2-, 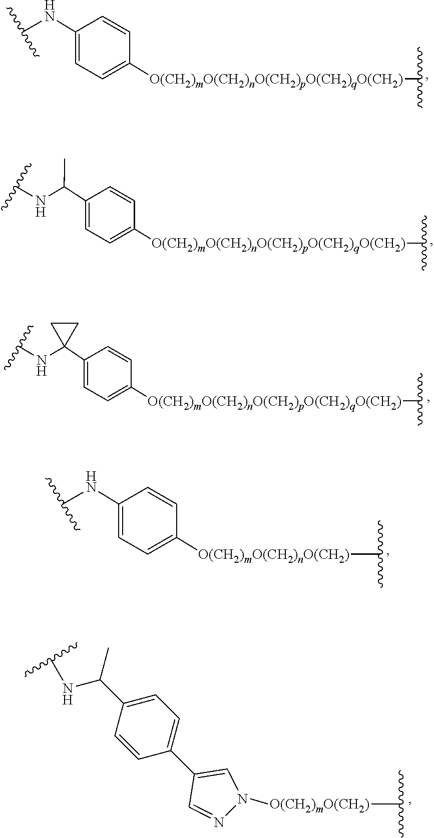
C(O)—(CH2)ₘ-O(CH2)ₙ-O(CH2)ₒ-O(CH2)ₚ-O(CH2)_{q}-O(CH2)ᵣ-OCH2-C(O), C(O)—(CH2)ₘ-O(CH2)ₙ-O(CH2)ₒ-O(CH2)ₚ-O(CH2)_{q}-OCH2-C(O), C(O)—(CH2)ₘ-O(CH2)ₙ-O(CH2)ₒ-O(CH2)ₚ-OCH2-C(O), C(O)—(CH2)ₘ-O(CH2)ₙ-O(CH2)ₒ-O(CH2)ᵣ-OCH2-C(O), or C(O)—(CH2)ₘ-OCH2-C(O),
wherein m, n, o, p, q, and r are 1, 2, 3, or 4.

49. The compound of claim 2, wherein the linker is an optionally substituted C₁-C₁₀ alkyl, C₁-C₁₀ heteroalkyl, C₁-C₁₀ alkynyl, 3-12 membered carbocycle, or 3-12 membered heterocycle, wherein each C₁-C₁₀ alkyl, C₁-C₁₀ alkynyl, 3-12 membered carbocycle, or 3-12 membered heterocycle of L² is optionally substituted with LBM, and/or one or more groups of R^{A10};
Wherein R^{A10} is C₁-C₁₀ alkyl, C₁-C₁₀ alkynyl, 3-12 membered carbocycle, or 3-12 membered heterocycle is independently optionally substituted with LBM, and/or one or more of the substituent groups selected from CH₃, OH, OCH₃ and F.

50. The compound of claim 2, wherein the linker has the structure:

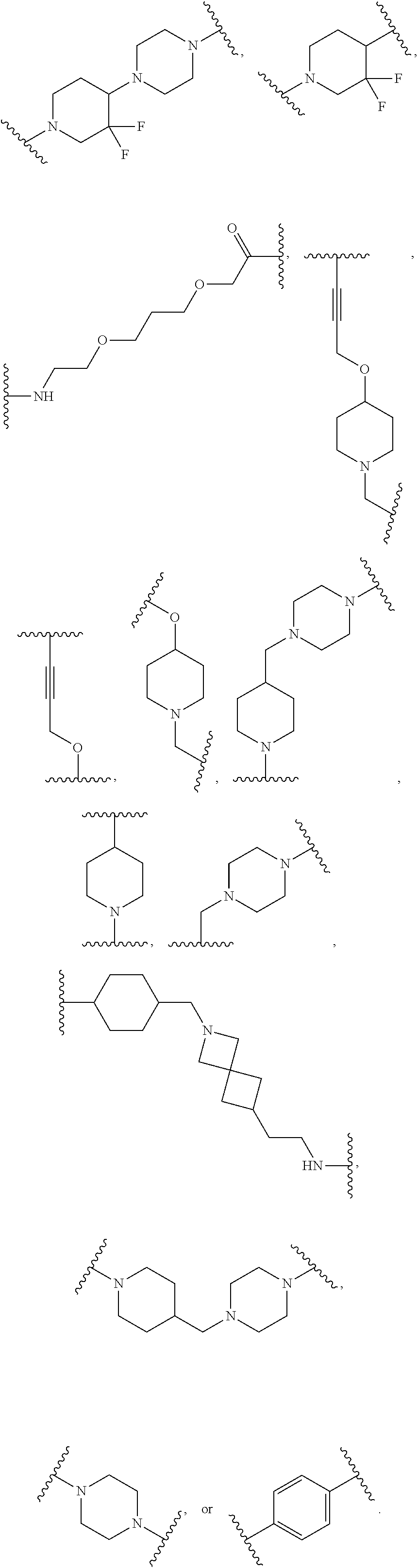

51. The compound of claim 2, wherein the targeting ligand binds to BRD9, CBP, EP300, estrogen receptor, androgen receptor, IRAK4, IRAK4 and/or MiD, STAT3, MDM2, BCLXL, BTK, BTK and/or MiD, TRK, BRM, IKZF1 and/or IKZF3, BRAF V600x, or EGFR L858R.

52. A pharmaceutically acceptable composition comprising a long-acting formulation of a compound of any one of claims 1 to 2 that reduces the level and/or activity of BRD9, or a pharmaceutically acceptable salt thereof, and a pharmaceutically acceptable excipient.

53. A pharmaceutically acceptable composition comprising a long-acting formulation of a compound of any one of claims 1 to 2 that reduces the level and/or activity of CBP, or a pharmaceutically acceptable salt thereof, and a pharmaceutically acceptable excipient.

54. A pharmaceutically acceptable composition comprising a long-acting formulation of a compound of any one of claims 1 to 2 that reduces the level and/or activity of EP300, or a pharmaceutically acceptable salt thereof, and a pharmaceutically acceptable excipient.

55. A pharmaceutically acceptable composition comprising a long-acting formulation of a compound of any one of claims 1 to 2 that reduces the level and/or activity of estrogen receptor, or a pharmaceutically acceptable salt thereof, and a pharmaceutically acceptable excipient.

56. A pharmaceutically acceptable composition comprising a long-acting formulation of a compound of any one of claims 1 to 2 that reduces the level and/or activity of androgen receptor, or a pharmaceutically acceptable salt thereof, and a pharmaceutically acceptable excipient.

57. A pharmaceutically acceptable composition comprising a long-acting formulation of a compound of any one of claims 1 to 2 that reduces the level and/or activity of IRAK4, or a pharmaceutically acceptable salt thereof, and a pharmaceutically acceptable excipient.

58. A pharmaceutically acceptable composition comprising a long-acting formulation of a compound of any one of claims 1 to 2 that reduces the level and/or activity of IRAK4 and/or IMiD, or a pharmaceutically acceptable salt thereof, and a pharmaceutically acceptable excipient.

59. A pharmaceutically acceptable composition comprising a long-acting formulation of a compound of any one of claims 1 to 2 that reduces the level and/or activity of STAT3, or a pharmaceutically acceptable salt thereof, and a pharmaceutically acceptable excipient.

60. A pharmaceutically acceptable composition comprising a long-acting formulation of a compound of any one of claims 1 to 2 that reduces the level and/or activity of MDM2, or a pharmaceutically acceptable salt thereof, and a pharmaceutically acceptable excipient.

61. A pharmaceutically acceptable composition comprising a long-acting formulation of a compound of any one of claims 1 to 2 that reduces the level and/or activity of BCLXL, or a pharmaceutically acceptable salt thereof, and a pharmaceutically acceptable excipient.

62. A pharmaceutically acceptable composition comprising a long-acting formulation of a compound of any one of claims 1 to 2 that reduces the level and/or activity of BTK, or a pharmaceutically acceptable salt thereof, and a pharmaceutically acceptable excipient.

63. A pharmaceutically acceptable composition comprising a long-acting formulation of a compound of any one of claims 1 to 2 that reduces the level and/or activity of BTK and/or MiD, or a pharmaceutically acceptable salt thereof, and a pharmaceutically acceptable excipient.

64. A pharmaceutically acceptable composition comprising a long-acting formulation of a compound of any one of claims 1 to 2 that reduces the level and/or activity of TRK, or a pharmaceutically acceptable salt thereof, and a pharmaceutically acceptable excipient.

65. A pharmaceutically acceptable composition comprising a long-acting formulation of a compound of any one of claims 1 to 2 that reduces the level and/or activity of BRM, or a pharmaceutically acceptable salt thereof, and a pharmaceutically acceptable excipient.

66. A pharmaceutically acceptable composition comprising a long-acting formulation of a compound of any one of claims 1 to 2 that reduces the level and/or activity of IKZF1 and/or IKZF3, or a pharmaceutically acceptable salt thereof, and a pharmaceutically acceptable excipient.

67. A pharmaceutically acceptable composition comprising a long-acting formulation of a compound of any one of claims 1 to 2 that reduces the level and/or activity of BRAF V600x, or a pharmaceutically acceptable salt thereof, and a pharmaceutically acceptable excipient.

68. A pharmaceutically acceptable composition comprising a long-acting formulation of a compound of any one of claims 1 to 2 that reduces the level and/or activity of EGFR L585R, or a pharmaceutically acceptable salt thereof, and a pharmaceutically acceptable excipient.

69. A pharmaceutically acceptable composition comprising a long-acting formulation of the compound of any one of claims 1 to 2 having the structure: 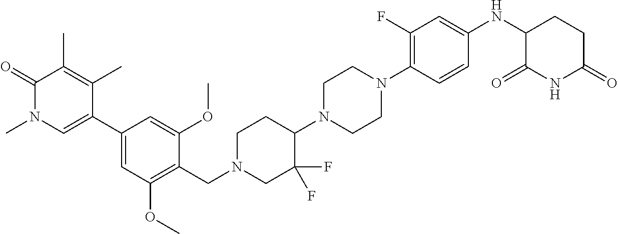 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1]
or a pharmaceutically acceptable salt thereof, and a pharmaceutically acceptable excipient.

70. A pharmaceutically acceptable composition comprising a long-acting formulation of the compound of any one of claims 1 to 2 having the structure: 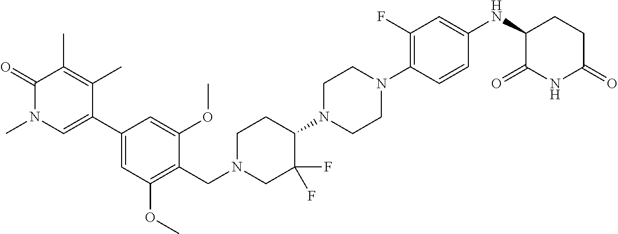 [SMILES: COc1cc(-c2cn(C)c(=O)c(C)c2C)cc(OC)c1CN1CC[C@H](N2CCN(c3ccc(N[C@H]4CCC(=O)NC4=O)cc3F)CC2)C(F)(F)C1]
or a pharmaceutically acceptable salt thereof, and a pharmaceutically acceptable excipient.

71. A pharmaceutically acceptable composition comprising a long-acting formulation of the compound of any one of claims 1 to 2 having the structure: 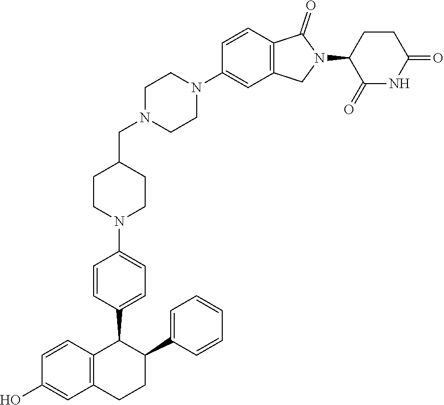 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, and a pharmaceutically acceptable excipient.

72. A pharmaceutically acceptable composition comprising a long-acting formulation of the compound of any one of claims 1 to 2 having the structure: 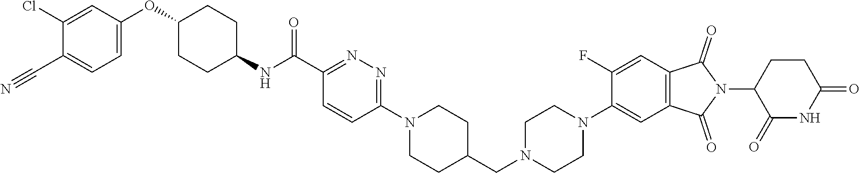 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
or a pharmaceutically acceptable salt thereof, and a pharmaceutically acceptable excipient.

73. A pharmaceutically acceptable composition comprising a long-acting formulation of the compound of any one of claims 1 to 2 having the structure: 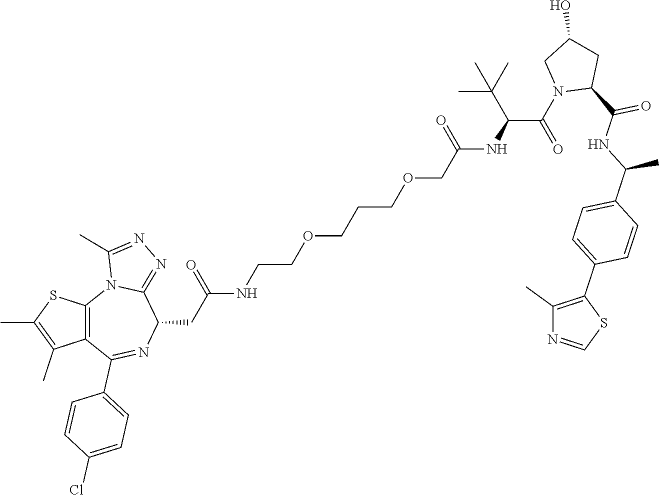 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1]
or a pharmaceutically acceptable salt thereof, and a pharmaceutically acceptable excipient.

74. A pharmaceutically acceptable composition comprising a long-acting formulation of the compound of any one of claims 1 to 2 having the structure: 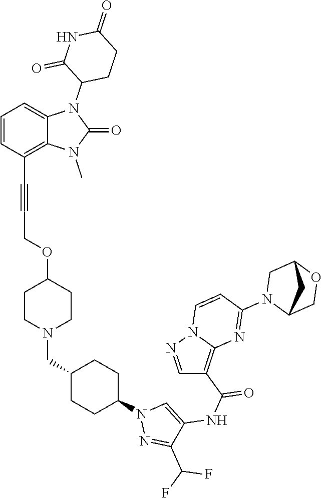 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]
or a pharmaceutically acceptable salt thereof, and a pharmaceutically acceptable excipient.

75. A pharmaceutically acceptable composition comprising a long-acting formulation of the compound of any one of claims 1 to 2 having the structure:
or a pharmaceutically acceptable salt thereof, and a pharmaceutically acceptable excipient.

76. A pharmaceutically acceptable composition comprising a long-acting formulation of the compound of any one of claims 1 to 2 having the structure: 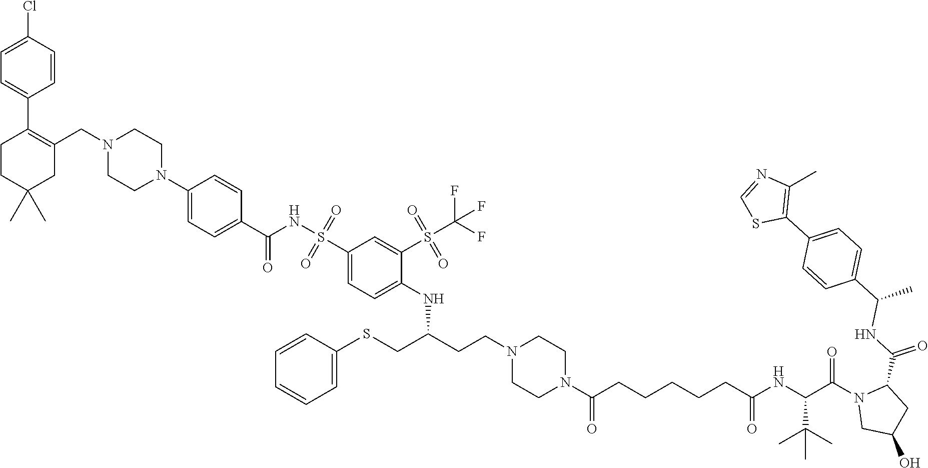 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]
or a pharmaceutically acceptable salt thereof, and a pharmaceutically acceptable excipient.

77. A pharmaceutically acceptable composition comprising a long-acting formulation of the compound of any one of claims 1 to 2 having the structure: 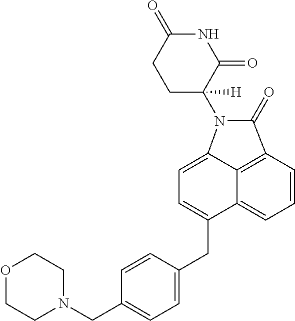 [SMILES: O=C1CC[C@H](N2C(=O)c3cccc4c(Cc5ccc(CN6CCOCC6)cc5)ccc2c34)C(=O)N1]
or a pharmaceutically acceptable salt thereof, and a pharmaceutically acceptable excipient.

78. The composition of any one of claims 1-2, wherein the excipient includes a solvent, a diluent, a granulating agent, a dispersing agent, a surfactant, an emulsifier, a stabilizer, a polymer, a binding agent, a preservative, a cryoprotectant, a buffering agent, a lubricating agent, an oil, or a combination thereof.

79. The composition of claim 78, wherein the excipient is a solvent.

80. The composition of any one of claims 78-79, wherein the solvent is water, dichloromethane, dimethyl sulfoxide, dimethyl formamide, ethyl acetate, methyl ethyl ketone, tetrahydrofuran, ethyl benzoate, chlorobenzene, benzyl alcohol, methyl n-propyl ketone, n-butyl acetate, trichloroethylene, ethyl-L-lactate, 2-methyl tetrahydrofuran, 1,2-dichlorobenzene, toluene, methyl isobutyl ketone, butyl lactate, p-xylene, acetone, or a combination thereof.

81. The composition of claim 78, wherein the excipient is a diluent.

82. The composition of any one of claims 78-81, wherein the diluent is calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, saline, dry starch, cornstarch, powdered sugar, or a combination thereof.

83. The composition of claim 78, wherein the excipient is a surfactant.

84. The composition of any one of claims 78-83, wherein the surfactant is polyvinylpyrrolidone (PVP), sodium carboxymethyl cellulose (CMC Na), PEG 4000, TWEEN®20 (polysorbate 20), TWEEN®80 (polysorbate 80), POLOXAMER®188, POLOXAMER®407, monooleate [SPAN®80], or a combination thereof.

85. The composition of claim 78, wherein the excipient is a polymer.

86. The composition of any one of claims 78-85, wherein the polymer is poly(urethane), poly(siloxane), poly(silicone), poly(methyl methacrylate), poly(vinyl alcohol), poly(ethylene), poly(vinyl pyrrolidone) (PVP), poly(vinyl pyrrolidone) K12 (e.g., PVP K12, polyvidone, or povidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactide (PLA), polyglycolide (PGA), poly(lactide-co-glycolide) (PLGA), polyanhydrides, polyorthoester, and/or a combination thereof.

87. The composition of claim 78, wherein the excipient is a cryoprotectant.

88. The composition of any one of claims 78-87, wherein the cryoprotectant is mannitol, PVP, PVA, PEG, amino acids such as glycine, glycerol, propylene glycol, dimethyl sulfoxide (DMSO), ethylene glycol, 2-methyl-2,4-pentadiol (MPD) or hexylene glycol, sucrose, trehalose, or a combination thereof.

89. The composition of any one of claims 1-88, wherein the formulation is lyophilized.

90. The composition of any one of claims 1-88, wherein the formulation is frozen.

91. The composition of any one of claims 1-88, wherein the formulation is a solution.

92. The composition of any one of claims 1-88, wherein the formulation is a suspension.

93. The composition of any one of claims 1-88, wherein the formulation is aqueous.

94. The composition of any one of claims 1-88, wherein the formulation is oil-based.

95. The composition of any one of claims 1-94, wherein the formulation is crystalline.

96. The composition of any one of claims 1-94, wherein the formulation is amorphous.

97. The composition of any one of claims 1-96, wherein the formulation is a nanosuspension.

98. The composition of claim 97, wherein the nanosuspension is aqueous.

99. The composition of claim 97, wherein the nanosuspension is oil based.

100. The composition of claim 97, wherein the nanosuspension comprises sucrose acetate isobutyrate (SAIB).

101. The composition of any one of claims 97-100, wherein the formulation comprises an average particle size of between about 100 nm to about 1000 nm.

102. The composition of any one of claims 1-96, wherein the formulation is a microsuspension.

103. The composition of claim 102, wherein the microsuspension is aqueous.

104. The composition of claim 102, wherein the microsuspension is oil based.

105. The composition of claim 104, wherein the oil is olive oil, soybean oil, sesame oil, or a combination thereof.

106. The composition of any one of claims 102-105, wherein the microsuspension has been prepared using a jet mill.

107. The composition of any one of claims 102-106, wherein the formulation comprises an average particle size of between about 1 μm to about 50 μm.

108. The composition of any one of claims 105-107, wherein the formulation has a viscosity that is between about 1 cp and about 25 cp.

109. The composition of any one of claims 97-108, wherein the formulation comprises TWEEN®20 (polysorbate 20).

110. The composition of any one of claims 97-109, wherein the formulation comprises sodium carboxymethyl cellulose (CMC Na).

111. The composition of any one of claims 97-110, wherein the formulation comprises poly(vinyl pyrrolidone) (PVP).

112. The composition of any one of claims 97-110, wherein the formulation comprises poly(vinyl pyrrolidone) K12 (PVP K12).

113. The composition of any one of claims 97-104 or 108, wherein the formulations comprises dimethyl sulfoxide (DMSO).

114. The composition of any one of claims 97-104 or 108, wherein the formulation comprises sorbitan monooleate [SPAN®80].

115. The composition of any one of claims 97-114, wherein the formulation comprises saline.

116. The composition of any one of claims 97-115, wherein the formulation comprises TWEEN®20 (polysorbate 20) and saline.

117. The composition of any one of claims 1-116, wherein the formulation comprises poly (lactic-co-glycolic) acid (PLGA).

118. The composition of claim 117, wherein the PLGA has an acid N-terminus.

119. The composition of any one of claims 1-96, wherein the formulation comprises poly (lactic-co-glycolic) acid (PLGA) microspheres.

120. The composition of claim 119, wherein the PLGA microspheres are between about 1 and about 100 microns in size.

121. The composition of any one of claims 119-120, wherein the PLGA microspheres are between about 5 and about 100 microns in size.

122. The composition of any one of claims 119-121, wherein the PLGA microspheres are suspended and dispersed in an excipient.

123. The composition of claim 122, wherein the excipient is a solvent, diluent, surfactant, or a combination thereof.

124. The composition of any one of claims 122-123, wherein the microspheres are suspended and dispersed in poly(vinyl alcohol) (PVA).

125. The composition of any one of claims 122-124, wherein the microspheres are suspended and dispersed in POLOXAMER®188 (P-188).

126. The composition of any one of claims 122-125, wherein the microspheres are suspended and dispersed in POLOXAMER®407 (P-407).

127. The composition of any one of claims 119-126, wherein the PLGA microsphere formulation has a viscosity of between about 1 cp and about 25 cp.

128. The composition of any one of claims 1-127, wherein the formulation comprises in situ PLGA gel.

129. The composition of any one of claims 119-128, wherein the formulation comprises a PLGA ratio of lactic acid to glycolic acid.

130. The composition of claim 129, wherein the PLGA ratio is 50% lactic acid to 50% glycolic acid.

131. The composition of claim 129, wherein the PLGA ratio is 65% lactic acid to 35% glycolic acid.

132. The composition of claim 129, wherein the PLGA ratio is 75% lactic acid to 25% glycolic acid.

133. The composition of claim 129, wherein the PLGA ratio is 85% lactic acid to 15% glycolic acid.

134. The composition of any one of claims 119-133, wherein the formulation comprises between about 80 mg and about 900 mg of PLGA per g of formulation.

135. The composition of any one of claims 119-133, wherein the formulation comprises between about 80 mg and about 200 mg of PLGA per g of formulation.

136. The composition of any one of claims 119-135, wherein the PLGA has an ester N-terminus.

137. The composition of any one of claims 119-135, wherein the PLGA has an acid N-terminus.

138. The composition of any one of claims 119-137, wherein the formulation comprises dimethylacetamide (DMA).

139. The composition of any one of claims 119-138, wherein the formulation comprises dimethyl sulfoxide (DMSO).

140. The composition of any one of claims 119-139, wherein the formulation comprises dimethylformamide (DMF).

141. The composition of any one of claims 119-140, wherein the formulation comprises N-methyl-2-pryrrolidone (NMP).

142. The composition of any one of claims 1-141, wherein the formulation comprises an in situ sucrose acetate isobutyrate (SAIB) gel.

143. A method of treating cancer in a subject in need thereof, the method comprising administering to the subject an effective amount of a composition of any one of claims 1-142, or a pharmaceutically acceptable salt thereof.

144. The method of claim 143, wherein the cancer is synovial sarcoma

145. A method of treating synovial sarcoma in a subject in need thereof, the method comprising administering to the subject an effective amount of a composition of any one of claims 1-142, or a pharmaceutically acceptable salt thereof.

146. The method of any one of claims 143-145, wherein the administering comprises combining the compound with an excipient to form a solution and administering the resulting solution.

147. The method of any one of claims 143-145, wherein the administering comprises coupling a vial comprising the compound and a syringe comprising an excipient, transferring the contents of the syringe into the vial, mixing to form a solution, and administering the resulting solution.

148. The method of any one of claims 143-145, wherein the administering comprises coupling a syringe comprising the compound and a syringe comprising an excipient, mixing to form a solution, and administering the resulting solution.

149. The method of any one of claims 143-145, wherein the administering comprises coupling a vial comprising the compound and a syringe comprising PLGA and an excipient, transferring the contents of the syringe into the vial, mixing to form a solution, and administering the resulting solution.

150. The method of any one of claims 143-145, wherein the administering comprises coupling a syringe comprising the compound and a syringe comprising PLGA and an excipient, mixing to form a solution, and administering the resulting solution.

151. The method of any one of claims 143-145, wherein the administering comprises mixing the formulation to form a suspension and administering the resulting suspension.

152. The method of any one of claims 143-145, wherein the administering comprises mixing the formulation to form a nanosuspension and administering the resulting nanosuspension.

153. The method of any one of claims 143-145, wherein the administering comprises mixing the formulation to form a microsuspension and administering the resulting microsuspension.

154. The method of any one of claims 143-153, wherein the effective amount of the compound is administered at a dose of between about 0.1 mg/kg and about 1000 mg/kg.

155. The method of any one of claims 143-154, wherein the effective amount of the compound is administered at a dose of between about 0.1 mg/mL and about 1000 mg/mL.

156. The method of any one of claims 143-155, wherein the effective amount of the compound is administered at a dose of between about 0.1 mg and about 1000 mg.

157. The method of any one of claims 143-156, wherein the formulation is administered in a dose of between about 0.25 mL and about 3 mL.

158. The method of any one of claims 143-157, wherein the effective amount of the compound is administered in a dose of between about 0.1 mg/m² and about 1000 mg/m².

159. The method of any one of claims 143-158, wherein the formulation is administered at least once every week.

160. The method of any one of claims 143-158, wherein the formulation is administered at least once every two weeks.

161. The method of any one of claims 143-158, wherein the formulation is administered at least once every three weeks.

162. The method of any one of claims 143-158, wherein the formulation is administered at least once every four weeks.

163. The method of any one of claims 143-158, wherein the formulation is administered at least once every month.

164. The method of any one of claims 143-158, wherein the formulation is administered at least once every six weeks.

165. The method of any one of claims 143-158, wherein the formulation is administered at least once every eight weeks.

166. The method of any one of claims 143-158, wherein the formulation is administered at least once every 2 months.

167. The method of any one of claims 143-158, wherein the formulation is administered to the subject in a one-week dosing cycle.

168. The method of any one of claims 143-158, wherein the formulation is administered to the subject in a two-week dosing cycle.

169. The method of any one of claims 143-158, wherein the formulation is administered to the subject in a three-week dosing cycle.

170. The method of any one of claims 143-158, wherein the formulation is administered to the subject in a four-week dosing cycle.

171. The method of any one of claims 143-158, wherein the formulation is administered to the subject in a one-month dosing cycle.

172. The method of any one of claims 143-158, wherein the formulation is administered to the subject in a six-week dosing cycle.

173. The method of any one of claims 143-158, wherein the formulation is administered to the subject in an eight-week dosing cycle.

174. The method of any one of claims 143-158, wherein the formulation is administered to the subject in a two-month dosing cycle.

175. The method of any one of claims 143-174, wherein the formulation is administered subcutaneously.

176. The method of any one of claims 143-174, wherein the formulation is administered intramuscularly.
